SEQUENCE LISTING (16 sequences)

SEQ ID NO: 1 - DNA, 20 bp, Artificial Sequence
  human GAPDH forward PCR primer
cgctctctgctcctcctgtt

SEQ ID NO: 2 - DNA, 20 bp, Artificial Sequence
  human GAPDH reverse PCR primer
ccatggtgtctgagcgatgt

SEQ ID NO: 3 - DNA, 20 bp, Artificial Sequence
  bovine GAPDH forward PCR primer
agatggtgaaggtcggagtg

SEQ ID NO: 4 - DNA, 20 bp, Artificial Sequence
  bovine GAPDH reverse PCR primer
gatctcgctcctggaagatg

SEQ ID NO: 5 - DNA, 23 bp, Artificial Sequence
  human aggrecan forward primer
tgaggagggctggaacaagtacc

SEQ ID NO: 6 - DNA, 23 bp, Artificial Sequence
  human aggrecan reverse PCR primer
ggaggtggtaattgcagggaaca

SEQ ID NO: 7 - DNA, 20 bp, Artificial Sequence
  bovine aggrecan forward PCR primer
gtggggcaagactatgatcg

SEQ ID NO: 8 - DNA, 21 bp, Artificial Sequence
  bovine aggrecan reverse PCR primer
gcttgctcctccactaatgtc

SEQ ID NO: 9 - DNA, 20 bp, Artificial Sequence
  human COL2A1 forward PCR primer
tcacgtacactgccctgaag

SEQ ID NO: 10 - DNA, 20 bp, Artificial Sequence
  human COL2A1 reverse PCR primer
ttgcaacggattgtgttgtt

SEQ ID NO: 11 - DNA, 20 bp, Artificial Sequence
  bovine COL2A1 forward PCR primer
tcacgtacactgccctgaag

SEQ ID NO: 12 - DNA, 20 bp, Artificial Sequence
  bovine COL2A1 reverse PCR primer
tgcaatggattgtgttggtt

SEQ ID NO: 13 - DNA, 24 bp, Artificial Sequence
  human COL1A2 forward PCR primer
agggcaacagcaggttcacttaca

SEQ ID NO: 14 - DNA, 24 bp, Artificial Sequence
  human COL1A2 reverse PCR primer
agcgggggaaggagttaatgaaac

SEQ ID NO: 15 - DNA, 20 bp, Artificial Sequence
  bovine COL1A2 forward primer
acattggcccagtctgtttc

SEQ ID NO: 16 - DNA, 20 bp, Artificial Sequence
  bovine COL1A2 reverse PCR primer
gggagggggagtgaattaaa

(19) United States
(12) Patent Application Publication
(10) Pub. No.: US 2016/0228610 A1
(43) Pub. Date: Aug. 11, 2016

(54) TISSUE ENGINEERING USING PROGENITOR CELLS TO CATALYZE TISSUE FORMATION BY PRIMARY CELLS

(71) Applicant: The Board of Trustees of the Leland Stanford Junior University, Palo Alto, CA (US)

(72) Inventors: Janice Lai, Mountain View, CA (US); William Maloney, Menlo Park, CA (US); R. Lane Smith, Palo Alto, CA (US); Fan Yang, Stanford, CA (US)

(21) Appl. No.: 15/137,439

(22) Filed: Apr. 25, 2016

Related U.S. Application Data
(63) Continuation-in-part of application No. 14/173,628, filed on Feb. 5, 2014.
(60) Provisional application No. 61/761,121, filed on Feb. 5, 2013.

(51) Int. Cl. A61L 27/38; A61K 35/28; A61K 47/10; A61L 27/36; A61K 9/14; A61L 27/54; A61L 27/52; A61K 35/32; A61K 38/18

(52) U.S. Cl. CPC A61L 27/3834; A61K 35/32; A61K 35/28; A61K 47/10; A61K 38/1841; A61K 9/14; A61L 27/54; A61L 27/52; A61L 27/3654; A61L 27/3817; A61L 2430/06

(57) ABSTRACT

Methods of regenerating tissue using progenitor cells in combination with primary cells from a target tissue are disclosed. The progenitor cells catalyze proliferation and tissue production by primary cells allowing the use of fewer primary cells from a target tissue for effective tissue regeneration. Cell-based therapies combining progenitor cells and primary cells can be used for repair and regeneration of damaged tissue and organs for treating bodily injuries and degenerative diseases. In particular, adipose-derived stem cells and chondrocytes, co-encapsulated in a mixed culture in a polyethylene glycol-based hydrogel comprising an extracellular matrix molecule such as chondroitin sulfate methacrylate, hyaluronic acid methacrylate, or heparan sulfate methacrylate, effectively produce cartilage that can be used for treatment of traumatic injuries or diseases involving cartilage degeneration.

### CROSS-REFERENCE TO RELATED APPLICATION

This application is a continuation-in-part of U.S. application Ser. No. 14/173,628, filed Feb. 5, 2014, which claims benefit under 35 U.S.C. §119(e) of provisional application 61/761,121, filed Feb. 5, 2013, all of which applications are hereby incorporated herein by reference in their entireties.

### TECHNICAL FIELD

The present invention pertains generally to tissue engineering and regenerative medicine. In particular, the invention relates to methods of regenerating tissue using progenitor cells to catalyze proliferation and tissue production by primary cells.

### BACKGROUND

Cell-based therapy is a promising strategy for tissue repair and regeneration. In particular, primary cells, originating from the same tissue type as the damaged tissue in need of regeneration, possess the right phenotype for use in tissue replacement; however, the scarcity of available primary cells is a major hurdle preventing the widespread application of primary cell-based therapy for tissue repair. Furthermore, primary cells often cannot proliferate in vitro or they rapidly de-differentiate during expansion in vitro, further hindering their clinical application.

Cell-based approaches, for example, are sought after for cartilage repair and regeneration. Cartilage damage and loss is prevalent among adults and the older population, and can be caused by traumatic injury or degenerative diseases, such as arthritis. Due to its avascular nature, articular cartilage has limited self-repair potential (Mankin et al. (1982) J. Bone Joint Surg. Am. 64:460-466). Furthermore, the proliferation and regeneration potential of chondrocytes declines with age (Barbero et al. (2004) Osteoarthritis Cartilage 12:476-484). Damage to cartilage is often irreversible and if not treated properly, may alter mechanical loading and lead to the early onset of osteoarthritis (Griffin et al. (2005) Exerc. Sport Sci. Rev. 33:195-200).

Cell-based approaches using allogeneic neonatal chondrocytes offer a promising solution to cartilage regeneration. Neonatal chondrocytes, unlike other commonly used cell sources, such as autologous chondrocytes or mesenchymal stem cells from bone marrow, are highly proliferative, immune-privileged, and can readily produce abundant cartilage matrix, making neonatal chondrocytes a superior cell source for cartilage regeneration (Adkisson et al. (2001) Clin. Orthop. Relat. Res. 2001:S280-294; Adkisson et al. (2010) Stem Cell Res. 4:57-68; and Adkisson et al. (2010) Am. J. Sports Med. 38:1324-1333). However, the scarcity of neonatal chondrocytes and their rapid de-differentiation during expansion in vitro seriously hinders their clinical application.

There remains a need for improved cell-based therapies for repair and regeneration of damaged tissue and organs for treating bodily injuries and degenerative diseases.

### SUMMARY

The invention relates to cell-based therapies combining progenitor cells and primary cells for repair and regeneration of damaged tissue and organs for treating bodily injuries and degenerative diseases. The progenitor cells are used to induce primary cells to proliferate and enhance tissue production by co-culture of the two cell-types in a three-dimensional scaffold. In particular, adipose-derived stem cells and chondrocytes, co-encapsulated in mixed cultures in a hydrogel, provide robust cartilage regeneration while substantially reducing the percentage of chondrocytes needed to produce cartilage for treatment of traumatic injuries or diseases involving cartilage degeneration (see Examples 1-3).

Thus, in one aspect, the invention includes a composition comprising a three-dimensional scaffold encapsulating progenitor cells and tissue-specific primary cells. The scaffold should be biocompatible with the encapsulated cells and allows production of the desired product from the primary cells. In one embodiment, the scaffold is a biomimetic scaffold that mimics certain aspects of the natural cell environment of the primary cell, such as the structure and function of the extracellular matrix (ECM). For example, the scaffold may be a hydrogel, which binds to paracrine signaling molecules released from the encapsulated cells. The progenitor cells and tissue-specific primary cells can be combined in the three-dimensional scaffold as a mixed culture, in which the progenitor cells and primary cells are uniformly mixed. In the mixed culture, the ratio of the two cell types can be adjusted to achieve optimum production of the desired cell product. The use of progenitor cells to catalyze tissue production by the primary cells allows a smaller number of primary cells to be used for tissue production than would be needed if the primary cells were used alone in tissue production. In one embodiment, the number of tissue-specific primary cells used in compositions for tissue production is the minimal number needed to promote a therapeutically effective amount of tissue production to treat a particular injury or disease involving tissue degeneration. In certain embodiments, one or more additional factors, such as nutrients, cytokines, growth factors, or antibiotics may be added to the scaffold to improve cell function or viability. The composition may also further comprise a pharmaceutically acceptable carrier.

In certain embodiments, the invention includes a composition for generating new cartilage comprising adipose-derived stem cells and chondrocytes encapsulated in a hydrogel. In one embodiment, the hydrogel composition comprises a polyethylene glycol (PEG)-based hydrogel. Exemplary hydrogels include a poly(ethylene glycol) diacrylate (PEGDA) or poly(ethylene glycol) dimethacrylate (PEGDMA) hydrogel. In another embodiment, the hydrogel composition further comprises at least one extracellular matrix molecule, including, but not limited to, chondroitin sulfate methacrylate (CS-MA), hyaluronic acid methacrylate (HA-MA), and heparan sulfate methacrylate (HS-MA). In certain embodiments, at least one extracellular matrix molecule is present in the hydrogel at a concentration ranging from about 0.5% (w/v) to about 5% (w/v), or any concentration within this range, including 0.5, 0.75, 1.0, 1.25, 1.5, 1.75, 2.0, 2.25, 2.5, 2.75, 3.0, 3.25, 3.5, 3.75, 4.0, 4.25, 4.5, 4.75, or 5.0% (w/v). In certain embodiments, the hydrogel composition comprises PEGDMA at a concentration ranging from about 8% (w/v) to about 14% (w/v) or any concentration within this range, including 8, 8.5, 9, 9.5, 10, 10.5, 11, 11.5, 12, 12.5, 13, 13.5, or 14% (w/v). In certain embodiments, the hydrogel has a mechanical stiffness having a Young's modulus of from about 3 kPa to about 100 kPa, or any value within this range, including 3, 4, 5, 6, 7, 8, 9, 10, 11, 12, 13, 14, 15, 20, 25, 30, 35, 40, 45, 50, 55, 60, 65, 70, 75, 80, 85, 90, 95, or 100 kPa.

The adipose-derived stem cells and chondrocytes are combined as a mixed culture in the hydrogel. In certain embodiments, adipose-derived stem cells and chondrocytes are combined in a mixed culture, wherein the ratio of adipose-derived stem cells to chondrocytes is about 25:75, about 50:50, about 75:25, about 90:10, about 95:5, about 98:2, about 99:1, or any ratio in between. In another embodiment, the percentage of cells in the mixed culture that are chondrocytes is 1%-2%, 2%-5%, 5%-10%, 10-25%, or any percentage within these ranges, including 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%. In another embodiment, the percentage of chondrocytes in the mixed culture is 1% or less. In another embodiment, the number of chondrocytes is the minimal number needed to promote a therapeutically effective amount of cartilage production to treat an injury or disease involving cartilage degeneration.

The chondrocytes, so encapsulated, produce cartilage in an amount effective for treatment of a subject in need of repair or replacement of cartilage. Thus, compositions of the invention can be used for treating a subject for a traumatic injury or a disease involving cartilage degeneration. In one embodiment, the invention includes a method of treating a subject for cartilage damage or loss, the method comprising administering a therapeutically effective amount of a composition, described herein, comprising a mixed culture of adipose-derived stem cells and chondrocytes to the subject.

The chondrocytes used in treatment may be autologous or allogeneic. Preferably, the chondrocytes are derived from the patient or a matched donor. After transplantation of the hydrogel composition comprising the mixed culture to the patient, the chondrocytes in the hydrogel composition produce new cartilage in vivo. Such cartilage is capable of filling cartilage defects of any shape and size at the treatment site. The new cartilage can be produced in vivo even under hypoxic conditions, for example, wherein the local O₂ tension ranges from 1% to 7%.

In another embodiment, the invention includes a method for treating a patient for cartilage damage or loss, the method comprising: a) combining chondrocytes with adipose-derived stem cells in a mixed culture, wherein the mixed culture comprises 1% to 25% chondrocytes and 75% to 99% adipose-derived stem cells; b) adding the mixed culture to a hydrogel composition comprising chondrogenic media, TGF-β3, and at least one extracellular matrix molecule selected from the group consisting of chondroitin sulfate methacrylate (CS-MA), hyaluronic acid methacrylate (HA-MA), and heparan sulfate methacrylate (HS-MA), wherein the hydrogel composition has a Young's modulus of from about 3 kPa to about 100 kPa; and c) transplanting the hydrogel composition comprising the mixed culture to the patient at a site in need of cartilage replacement. In another embodiment, the method further comprises administering an effective amount of TGF-β3 to the patient after transplantation of the hydrogel to the patient.

In another embodiment, the hydrogel composition comprising the mixed culture is transplanted to the patient after culturing the chondrocytes ex vivo in the hydrogel composition for a period of time. For example, the chondrocytes can be cultured in a mixed culture with the adipose-derived stem cells for a few days or weeks, such as at least 1, day, 2 days, 3 days, 4 days, 5 days, 6 days, 1 week, 1.5 weeks, 2 weeks, 2.5 weeks, 3 weeks, or longer prior to transplantation of the hydrogel composition to the patient.

In another embodiment, the invention includes a method for producing cartilage, the method comprising: a) obtaining chondrocytes from a subject; b) combining the chondrocytes with adipose-derived stem cells in a mixed culture, wherein the mixed culture comprises 1% to 25% chondrocytes and 75% to 99% adipose-derived stem cells; c) adding the mixed culture to a hydrogel composition, wherein the hydrogel composition has a Young's modulus of from about 3 kPa to about 100 kPa; culturing the chondrocytes ex vivo or in vivo in the hydrogel composition, wherein the chondrocytes are cultured in the mixed culture with the adipose-derived stem cells in chondrogenic media comprising TGF-β3 and at least one extracellular matrix molecule selected from the group consisting of chondroitin sulfate methacrylate (CS-MA), hyaluronic acid methacrylate (HA-MA), and heparan sulfate methacrylate (HS-MA) under conditions, whereby cartilage is produced comprising nodules having a nodule size of at least 100 μm in length.

In another aspect, the invention includes a hydrogel composition comprising cartilage prepared by a method described herein.

In another aspect, the invention includes a method of preparing a hydrogel composition for generating new cartilage in a subject, wherein the composition comprises a mixed culture of adipose-derived stem cells and chondrocytes. The method comprises: a) mixing a PEG-based polymer (e.g., PEGDA or PEGDMA) and at least one extracellular matrix molecule (e.g., CS-MA, HA-MA, and HS-MA) with water; b) adding adipose-derived stem cells and chondrocytes and media suitable for growth of the adipose-derived stem cells and chondrocytes to form a suspension; and c) inducing crosslinking of the PEG-based polymer to form a hydrogel. In certain embodiments, the method further comprises culturing the adipose-derived stem cells and chondrocytes in the presence of TGF-β3 in the hydrogel under conditions in which the cells proliferate and produce cartilage before implantation of the composition in a subject.

The compositions described herein may be administered by any suitable method, such as by injection or implantation locally into an area of tissue damage or loss. For example, compositions, described herein, for treatment of cartilage loss or damage may be administered by injection or implantation locally into an area of cartilage damage or loss, such as a damaged joint of a subject.

In another aspect, the invention includes a kit comprising a composition for generating new tissue, as described herein, or reagents and cells for preparing such a composition (e.g., reagents for preparing a three-dimensional scaffold, progenitor cells, primary cells, media, and optionally one or more other factors, such as growth factors, ECM components, antibiotics, and the like). The kit may also comprise means for delivering the composition to a subject and instructions for treating a traumatic injury or a disease involving tissue degeneration.

In one embodiment, the invention includes a kit comprising a hydrogel composition for generating new cartilage, as described herein, or reagents and cells for preparing such a composition (e.g., TGF-β3, CS-MA, HA-MA, HS-MA, PEGDA, and/or PEGDMA), adipose-derived stem cells, chondrocytes, media, and optionally one or more other factors, such as growth factors, ECM components, antibiotics, and the like). The kit may also comprise means for delivering the composition to a subject and instructions for treating a traumatic injury or a disease involving cartilage degeneration.

In another embodiment, the invention includes a method of treating a patient for cartilage damage or loss, the method comprising producing cartilage by a method described herein, and transplanting the cartilage to the patient at a site in need thereof. The cartilage may be administered, for example, locally at a damaged joint of the subject to treat a subject having a traumatic injury or a disease involving cartilage degeneration (e.g., arthritis).

These and other embodiments of the subject invention will readily occur to those of skill in the art in view of the disclosure herein.

### BRIEF DESCRIPTION OF THE FIGURES

FIGS. 1A-1E show a schematic representation of the experimental design. In order to examine the interaction between adipose-derived stem cells (ADSCS) and neonatal chondrocytes (CHONS), three different in vitro culture models were used: conditioned medium (FIG. 1A)—cells were cultured with supplementation of conditioned medium from the other cell type (CM), bi-layer (FIG. 1B)—culture confined the two cell types to separate layers with no direct cell-cell contact, but allowing paracrine signals to diffuse into the adjacent layer, and mixed cell (FIG. 1C)—mixed cultures of the two cell types in one scaffold at different cell ratios. By changing the ratio of the two cell types in a 3D volume, we can tune the spatial distribution and distance between a chondrocyte from an ADSC, thereby changing the paracrine signal concentration they are sensing (FIG. 1D). Human adult ADSCS and bovine neonatal CHONS were encapsulated in 3D biomimetic hydrogels and cultured in vitro for 21 days in chondrogenic medium supplemented with TGF-β3. FIG. 1E shows that in the mixed cell culture, increasing ADSC ratio while keeping the overall cell density constant at 15 million/ml leads to a linear increase in the number of ADSCS that are within effective communication distance (250 μm) of a CHON.

FIGS. 2A-2F show the gene expression of encapsulated cells in the three different types of co-culture models as illustrated in FIGS. 1A-1C, including CM: conditioned medium, Bi: bi-layered, and mixed co-culture at various ratios ranging from 75C:25A to 10C: 90A (C: chondrocytes, A: ADSCS). To distinguish the fate of each cell type, specie-specific primers were used to identify the gene expression of human ADSCS and bovine CHONS in the xenogeneic culture. Human-specific (FIGS. 2A-2C) and bovine-specific (FIGS. 2D-2F) gene expression were compared at day 21 relative to day 1 ADSC and bAC controls. Conditioned medium treatment and bi-layered co-culture led to minimal changes in cartilage marker expression, including Aggrecan (Agg) and type II collagen (COL2). In contrast, all groups using mixed co-culture at all ratios led to about 6-fold higher expression of Agg and about 20-fold higher expression in COL2 by human ADSCS. Meanwhile, mixed co-culture also led to markedly decreased undesirable expression of the fibrocartilage marker, type I collagen (COL1) compared with the human ADSCS cultured alone (control). For bovine chondrocytes, mixed co-culture led to a maintained cartilage phenotype and slightly decreased expression of the fibrocartilage marker COL1. Conditioned medium and bi-layered co-culture led to a slight decrease in Agg and COL2 expression in chondrocytes.

FIGS. 3A-3G show biochemical analyses of cell proliferation, matrix production and mechanical properties of the cell-laden scaffolds by the end of the 21-day culture. Only mixed co-culture at various ratios, but not CM or bi-layered culture, led to markedly enhanced cell proliferation and cartilage matrix production. FIG. 3A shows measurements of DNA content at day 1 and 21, which were used to evaluate cell proliferation over time. In the ADSC control group, DNA content at day 21 was reduced to 29% of day 1 DNA content (FIG. 3A). Both conditioned medium and bi-layer co-cultures had significantly higher numbers of ADSCS than that of ADSC control at day 21. To quantify cartilage matrix production, sulfated glycosaminoglycan (sGAG) content (FIG. 3B) and total collagen content (FIG. 3C) were measured at day 21. SGAG and collagen per wet weight exhibited similar trends. FIG. 3D shows compressive moduli of the cell-laden samples by the end of 21 days in culture. To compare the extent of cell number and matrix production changes as a result of variation in cell ratio, the interaction index, which is the measured matrix content normalized by the expected matrix content based on the matrix content was measured in the CHON and ADSC control groups. The interaction index for DNA/w.w. (FIG. 3E), GAG/w.w. (FIG. 3F), and collagen/w.w. (FIG. 3G) increased with an increase in ADSC ratio in the mixed cell culture. FIGS. 3E-3G show the effects of cell ratio variation on cell proliferation and cartilage matrix production. The measured DNA (FIG. 3E), sGAG (FIG. 3F), and collagen (FIG. 3G) were compared against expected values. At each cell ratio, the interaction index, which is the measured matrix content (DNA, sGAG, or collagen) in the mixed co-culture group normalized by the expected matrix content, based on the measured matrix content in the CHON and hADSC alone groups, was calculated. The interaction for DNA, sGAG, and collagen per wet weight in all the mixed co-culture groups were higher than 1.

FIGS. 4A-4F show type II collagen (COL2) immunostaining in the three cell co-culture models. The differential effects of conditioned medium (CM), bi-layer (Bi), and mixed cell culture on cartilage matrix production were evident in the spatial organization of neo-cartilage within the 3D hydrogels as shown by the type II collagen immunostaining (FIG. 4A). Conditioned medium and bi-layer cultures did not show obvious changes in type II collagen production for either cell type. In contrast, mixed co-culture with all ratios led to formation of neo-cartilage nodules within the 3D hydrogels, with increasing size of each nodule as the ratio of ADSCS increased (FIG. 4A). To determine the distribution of the two cell types in the mixed cell cultures, ADSCS were membrane-labeled (red) prior to encapsulation in the hydrogels; FIG. 4B shows co-localization of type II collagen (top row) with labeled ADSCS (middle row) along with DAPI nuclei staining (bottom row). It was revealed that ADSCs were not present in cartilage nodules. Scale bars, 100 μm. FIGS. 4C-4E show the quantification of type II collagen immunostaining images, including cartilage nodule size at different ratios of ADSC at day 7 (FIG. 4C), day 14 (FIG. 4D), and day 21 (FIG. 4E), as well as the total percentage of area occupied by cartilage nodules at different cell ratios at days 7, 14, and 21 (FIG. 4F). Both the cartilage nodule size as well as the total area of hydrogel being replaced by cartilage nodules increased with an increase in ADSC ratio in the mixed cell culture.

FIGS. 5A-5D show histograms showing the distribution of intercellular distances between ADSCS and CHONS that are within effective communication distance (250 μm) from a CHON in a mixed cell culture with (FIG. 5A) 25% ADSC, (FIG. 5B) 50% ADSC, (FIG. 5C) 75% ADSC, and (FIG. 5D) 90% ADSC.

FIG. 6 shows Agg, COL1, and COL 2 expression in human ADSCS and bovine CHONS at day 1 and day 21 when cultured alone.

FIG. 7 shows the gross appearance of freeze-dried cell hydrogel constructs at day 1 and day 21 (scalebar=10 mm).

FIG. 8 shows immunostaining of type II collagen in conditioned medium, bi-layer, and mixed cell culture groups at days 7 (top row) and 14 (bottom row). Cells were evenly distributed in the hydrogel construct at day 7. At day 14, cell aggregates and cartilage nodules (type II collagen positive) were observed in all the mixed cell culture groups (scale bars=100 μm).

FIG. 9 shows immunostaining of type I collagen in conditioned medium, bi-layer, and mixed cell cultures at day 21. Type I collagen was stained minimally.

FIGS. 10A-10H show that as little as 2% NChons in mixed co-culture induce synergistic cellular interactions and led to comparable amount of cartilage ECM production as NChon control. FIG. 10A shows a schematic of the experimental design, showing that mixed populations of NChons (FIG. 10C) and ADSCs (FIG. 10A) were encapsulated in 3D biomimetic hydrogels and cultured in vitro for 21 days in chondrogenic medium with TGF-β3 (Materials and Methods). The (FIG. 10B) DNA, (FIG. 10C) sGAG, and (FIG. 10D) collagen content per wet weight were quantified at day 21. The interaction index, defined as the measured (FIG. 10E) DNA, (FIG. 10F) sGAG, and (FIG. 10G) collagen content normalized by the expected content (Materials and Methods), reflects interaction synergy. Data are presented as mean±standard deviation (n=3). *p<0.05 versus pure NChon control; **p<0.01 versus pure NChon control; ***p<0.001 versus pure NChon control; ̂p<0.05 versus pure ADSC control; ̂̂p<0.01 versus pure ADSC control; ̂̂̂p<0.001 versus pure ADSC control. FIG. 10H shows immunostaining against type II (top) and type I (bottom) collagen (green) with ADSCs (red) labeled in red fluorescent dye. Image labels reflect the ratio of NChons (FIG. 10C) to ADSCs (FIG. 10A). Cell nuclei were counterstained with 4′,6-diamidino-2-phenylindole (DAPI, blue). Note that the therapeutically desirable type II collagen is produced via co-culture with NChons and ADSCs. Scale bar=100 μm.

FIGS. 11A-11E show that TGF-β3 is required for catalyzed cartilage formation by mixed populations of ADSCs and NChons. The (FIG. 11A) DNA, (FIG. 11B) sGAG, and (FIG. 11C) collagen content per wet weight of cell-hydrogel constructs were quantified after 14 days of in-vitro culture in chondrogenic medium with or without 10 ng/mL TGF-β3 at 20% O₂. FIG. 11D shows interaction indices, which were calculated for DNA, sGAG, and collagen content per wet weight as a measure of interaction synergy. Data are presented as mean±standard deviation (n=3). *p<0.05 versus pure NChon control; **p<0.01 versus pure NChon control; ***p<0.001 versus pure NChon control; ̂p<0.05 versus pure ADSC control; ̂̂p<0.01 versus pure ADSC control; ̂̂̂p<0.001 versus pure ADSC control. +p<0.05; ++p<0.01; +++p<0.001. FIG. 11E shows that immunostaining against type II collagen (green) reveals that large cartilage nodules were formed only in mixed co-culture with TGF-β3 supplementation. Cell nuclei were counterstained with 4′,6-diamidino-2-phenylindole (DAPI, blue). Scale bar=100 μm.

FIGS. 12A-12E show that synergy between ADSCs and NChons persists under hypoxia. The (FIG. 12A) DNA, (FIG. 12B) sGAG, and (FIG. 12C) collagen content per wet weight of cell-hydrogel constructs were quantified after 14 days of in vitro culture in chondrogenic medium with 10 ng/mL TGF-β3 at 2 or 20% O₂. FIG. 12D shows interaction indices, which were calculated for DNA, sGAG, and collagen content per wet weight as a measure of interaction synergy. Data are presented as mean±standard deviation (n=3). *p<0.05 versus pure NChon control; **p<0.01 versus pure NChon control; ***p<0.001 versus pure NChon control; ̂p<0.05 versus pure ADSC control; ̂̂p<0.01 versus pure ADSC control; ̂̂̂p<0.001 versus pure ADSC control. +p<0.05; ++p<0.01; +++p<0.001. FIG. 12E shows that immunostaining against type II collagen (green) reveals that large cartilage nodules were formed only in mixed co-culture at both oxygen tension. Cell nuclei were counterstained with 4′,6-diamidino-2-phenylindole (DAPI, blue). Scale bar=100 μm.

FIGS. 13A-13G show that catalyzed cartilage formation by mixed population of NChons and ADSCs is sustained in vivo. FIG. 13A shows cell-hydrogel constructs containing 25% or 10% NChons or pure populations of NChons or ADSCs that were cultured in vitro for 14 days in chondrogenic medium with 10 ng/mL TGF-β3 prior to subcutaneous implantation into nude mice. At day 0 (white), week 3 (grey), and week 8 (black) after implantation, (FIG. 13B) DNA, (FIG. 13C) sGAG, and (FIG. 13D) collagen content per construct were evaluated. FIG. 13E shows compressive moduli of the cell-hydrogel constructs, which were also measured using unconfined compression testing. To quantify synergy in vivo, interaction indices for (FIG. 13F) sGAG and (FIG. 13G) collagen were calculated. Data are presented as mean±standard deviation (n=5 samples/group). *p<0.05; **p<0.01; ***p<0.001.

FIGS. 14A and 14B show that synergistic interactions between NChons and ADSCs result in expanded formation of neotissues in vivo with articular cartilage phenotype that gradually replace the original hydrogel matrices over 12 weeks. Newly deposited extracellular matrix was immunostained (green) against (FIG. 14A) type II collagen and (FIG. 14B) aggrecan at weeks 3, 8, and 12 after implantation. Cell nuclei were counterstained with 4′,6-diamidino-2-phenylindole (DAPI, blue). Scale bar=100 μm.

FIGS. 15A and 15B show live/dead viability staining 24 hours post-encapsulation. Cells remained viable 24 hours after encapsulation in 3D biomimetic hydrogels. Live (green)/dead (red) viability staining of (FIG. 15A) ADSCs and (FIG. 15B) NChons 24 hours after encapsulation in biomimetic hydrogels, indicating high viability.

FIGS. 16A and 16B show sGAG and collagen content per DNA under hypoxia. (DNA) in vitro. FIG. 16A shows sGAG and FIG. 16B shows collagen content per DNA of cell-hydrogel constructs after 14 days of in-vitro culture at 2% or 20% O₂. Data are presented as mean±standard deviation (n=3). *p<0.05 versus pure NChon control; **p<0.01 versus pure NChon control; ***p<0.001 versus pure NChon control; ̂p<0.05 versus pure ADSC control; ̂̂p<0.01 versus pure ADSC control; ̂̂̂p<0.001 versus pure ADSC control. +p<0.05; ++p<0.01; +++p<0.001.

FIGS. 17A and 17B show immunostaining of collagen type I and type X after 12 weeks in vivo. No fibroblastic or hypertrophic phenotypes are detected in neocartilage. Immunostaining (green) against (FIG. 17A) type I collagen and (FIG. 17B) type X collagen was performed at weeks 3, 8, and 12 after implantation. Cell nuclei were counterstained with 4′,6-diamidino-2-phenylindole (blue). Scale bar=100 μm.

FIG. 18 shows chemical composition of the hydrogel platform (39 combinatorial hydrogels). Three ECM molecules (CS-MA, HA-MA, and HS-MA) were chosen as biochemical cues. PEGDMA was used to control the mechanical stiffness of the hydrogels.

FIG. 19 shows Young's moduli of 39 combinatorial hydrogels with varying PEGDMA concentrations (8-14% (w/v)), different ECM types, and varying ECM concentrations (0.5-5% (w/v)). Dotted lines represent hydrogels containing PEGDMA only. The three PEGDMA concentrations yielded hydrogels with distinct mechanical stiffness. Dashed lines represent the mechanical stiffness of hydrogels without methacrylated ECM molecules.

FIGS. 20A-20I show biochemical assays quantifying DNA (FIGS. 20A-20C), sGAG (FIGS. 20D-20F), and collagen secretion (FIGS. 20G-20I) by NChons housed in combinatorial hydrogels after 21 days of in vitro culture under chondrogenic conditions. *p<0.05, **p<0.01, and ***p<0.001 versus control hydrogels without ECM components.

FIGS. 21A-21C show Young's modulus of cell-laden hydrogels containing 8% (w/v) (FIG. 21A), 11% (w/v) (FIG. 21B), and 14% (w/v) (FIG. 21C) PEGDMA after 21 days of in vitro culture under chondrogenic conditions.

FIGS. 22A and 22B show effects of mechanical stiffness and type of ECM on collagen II (FIG. 22A) or aggrecan (FIG. 22B) secretion as revealed by immunostaining. Only hydrogels containing 5% (w/v) CS-MA, HA-MA, or HS-MA are shown. Green: collagen II (FIG. 22A) or aggrecan (FIG. 22B). Blue: DAPI. Scale bars, 200 μm.

FIGS. 23A and 23B show effects of mechanical stiffness and type of ECM on collagen II (FIG. 23A) or aggrecan (FIG. 23B) secretion, as shown by immunostaining. Only hydrogels containing 0.5% (w/v) CS-MA, HA-MA, or HS-MA are shown. Green, collagen II (FIG. 23A) or aggrecan (FIG. 23B). Blue, DAPI. Scale bars, 200 μm.

FIG. 24 shows effects of mechanical stiffness and type of ECM on collagen I secretion, as indicated by immunostaining. Only hydrogels containing 5% (w/v) CS-MA, HA-MA, or HS-MA are shown. Green, collagen I. Blue, DAPI. Scale bars, 200 μm.

FIG. 25 shows NChon viability assayed using the LIVE/DEAD kit on day 1 and day 14 after encapsulation in 11% PEGDMA hydrogels. ECM molecules were added to the hydrogels as indicated. Scale bar, 400 μm.

FIGS. 26A-26F show biochemical assays to quantify collagen (FIGS. 26A-26C) and sGAG (FIGS. 26D-26F) production by cells after 21 days of in vitro culture under chondrogenic conditions. Collagen production and sGAG production by cells encapsulated in hydrogels containing the same ECM molecule are grouped for easy visualization of the dose response.

FIG. 27 shows effects of mechanical stiffness and type of ECM on collagen X secretion, as shown by immunostaining. Only hydrogels containing 5% (w/v) CS-MA, HA-MA, or HS-MA are shown. Note that the green channel was color-boosted five-fold versus the staining for collagen I and collagen II depicted in FIGS. 22-24 of the main text. Scale bars, 200 μm.

### DETAILED DESCRIPTION

The practice of the present invention will employ, unless otherwise indicated, conventional methods of medicine, biology, biomaterials science, pharmacology, chemistry, biochemistry, recombinant DNA techniques and immunology, within the skill of the art. Such techniques are explained fully in the literature. See, e.g., G. Vunjak-Novakovic and R. I. Freshney Culture of Cells for Tissue Engineering (Wiley-Liss, 1^{st} edition, 2006); Biomaterials Science: An Introduction to Materials in Medicine (B. D. Ratner, A. S. Hoffman, F. J. Schoen, J. E. Lemons eds., Academic Press, 2^{nd} edition, 2004); An Introduction to Biomaterials (Biomedical Engineering, J. O. Hollinger ed., CRC Press, 2^{nd} edition, 2011); Biomaterials Science: An Integrated Clinical and Engineering Approach (Y. Rosen and N. Elman eds., CRC Press, 1^{st} edition, 2012); Arthritis Research: Methods and Protocols, Vols. 1 and 2: (Methods in Molecular Medicine, Cope ed., Humana Press, 2007); Cartilage and Osteoarthritis (Methods in Molecular Medicine, M. Sabatini P. Pastoureau, and F. De Ceuninck eds., Humana Press; 2004); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); and Sambrook et al., Molecular Cloning: A Laboratory Manual (3^{rd} Edition, 2001).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a cell” includes a mixture of two or more cells, and the like.

The term “about,” particularly in reference to a given quantity, is meant to encompass deviations of plus or minus five percent.

As used herein, the term “conditioned medium” refers to a medium in which a specific cell or population of cells has been cultured, and then removed. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. The medium containing the cellular factors is the conditioned medium.

“Biocompatible” generally refers to a material and any metabolites or degradation products thereof that are generally non-toxic to the recipient and do not cause any significant adverse effects to the subject.

“Hydrogel” refers to a substance formed when an organic polymer (natural or synthetic) is cross-linked via covalent, ionic, or hydrogen bonds to create a three-dimensional open-lattice structure which entraps water molecules to form a gel. Biocompatible hydrogel refers to a polymer that forms a gel which is not toxic to living cells, and allows sufficient diffusion of oxygen and nutrients to the entrapped cells to maintain viability.

“Mammalian cell” refers to any cell derived from a mammalian subject suitable for transplantation into the same or a different subject. The cell may be xenogeneic, autologous, or allogeneic. The cell can be a primary cell obtained directly from a mammalian subject. The cell may also be a cell derived from the culture and expansion of a cell obtained from a subject. For example, the cell may be a stem cell. Immortalized cells are also included within this definition. In some embodiments, the cell has been genetically engineered to express a recombinant protein and/or nucleic acid.

The term “progenitor cell” refers to a cell which is capable of differentiating into a specific type of cell. Progenitor cells include, but are not limited to, progenitor cells from various types of tissues, such as mesenchymal stromal cells from bone marrow, endothelial progenitor cells, muscle progenitor cells (e.g., satellite cells), pancreatic progenitor cells, periosteum progenitor cells, neural progenitor cells, blast cells, intermediate progenitor cells, and stem cells, including stem cells from embryos, umbilical cord, or adult tissues, or induced pluripotent stem cells.

The term “stem cell” refers to a cell that retains the ability to renew itself through mitotic cell division and that can differentiate into a diverse range of specialized cell types. Mammalian stem cells can be divided into three broad categories: embryonic stem cells, which are derived from blastocysts, adult stem cells, which are found in adult tissues, and cord blood stem cells, which are found in the umbilical cord. In a developing embryo, stem cells can differentiate into all of the specialized embryonic tissues. In adult organisms, stem cells and progenitor cells act as a repair system for the body by replenishing specialized cells. Totipotent stem cells are produced from the fusion of an egg and sperm cell. Cells produced by the first few divisions of the fertilized egg are also totipotent. These cells can differentiate into embryonic and extraembryonic cell types. Pluripotent stem cells are the descendants of totipotent cells and can differentiate into cells derived from any of the three germ layers. Multipotent stem cells can produce only cells of a closely related family of cells (e.g., hematopoietic stem cells differentiate into red blood cells, white blood cells, platelets, etc.). Unipotent cells can produce only one cell type, but have the property of self-renewal, which distinguishes them from non-stem cells.

As used herein, the term “cell viability” refers to a measure of the amount of cells that are living or dead, based on a total cell sample. High cell viability, as defined herein, refers to a cell population in which greater than 85% of all cells are viable, preferably greater than 90-95%, and more preferably a population characterized by high cell viability containing more than 99% viable cells.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

“Transplant” refers to the transfer of a cell, tissue, or organ to a subject from another source. The term is not limited to a particular mode of transfer. Encapsulated cells may be transplanted by any suitable method, such as by injection or surgical implantation.

The term “arthritis” includes, but is not limited to, osteoarthritis, rheumatoid arthritis, lupus-associated arthritis, juvenile idiopathic arthritis, reactive arthritis, enteropathic arthritis and psoriatic arthritis.

The term “disease involving cartilage degeneration” is any disease or disorder involving cartilage and/or joint degeneration. The term “disease involving cartilage degeneration” includes disorders, syndromes, diseases, and injuries that affect spinal discs or joints (e.g., articular joints) in animals, including humans, and includes, but is not limited to, arthritis, chondrophasia, spondyloarthropathy, ankylosing spondylitis, lupus erythematosus, relapsing polychondritis, and Sjogren's syndrome.

By “therapeutically effective dose or amount” of a composition comprising progenitor cells and tissue-specific primary cells or a composition comprising primary cells and conditioned media from a culture comprising progenitor cells is intended an amount that, when administered as described herein, brings about a positive therapeutic response in a subject having tissue damage or loss, such as an amount that results in the generation of new tissue at a treatment site. The exact amount required will vary from subject to subject, depending on the species, age, and general condition of the subject, the severity of the condition being treated, mode of administration, and the like. An appropriate “effective” amount in any individual case may be determined by one of ordinary skill in the art using routine experimentation, based upon the information provided herein.

For example, a therapeutically effective dose or amount of a composition comprising adipose-derived stem cells and chondrocytes or a composition comprising chondrocytes and conditioned media from a culture comprising adipose-derived stem cells is intended an amount that, when administered as described herein, brings about a positive therapeutic response in a subject having cartilage damage or loss, such as an amount that results in the generation of new cartilage at a treatment site (e.g., a damaged joint). For example, a therapeutically effective dose or amount could be used to treat cartilage damage or loss resulting from a traumatic injury or a degenerative disease, such as arthritis or other disease involving cartilage degeneration. Preferably, a therapeutically effective amount restores function and/or relieves pain and inflammation associated with cartilage damage or loss.

The terms “subject,” “individual,” and “patient,” are used interchangeably herein and refer to any mammalian subject for whom treatment or therapy is desired, particularly humans. Other subjects may include cattle, dogs, cats, guinea pigs, rabbits, rats, mice, horses, and so on. In some cases, the methods of the invention find use in experimental animals, in veterinary application, and in the development of animal models for disease, including, but not limited to, rodents including mice, rats, and hamsters; and primates.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention relates to methods of regenerating tissue using progenitor cells in combination with primary cells from a target tissue. In particular, progenitor cells catalyze proliferation and tissue production by primary cells allowing the use of fewer primary cells from a target tissue for effective tissue regeneration. The use of progenitor cells in combination with primary cells is highly advantageous given the scarcity of available primary cells, the inability of many primary cells to proliferate, and their tendency to rapidly de-differentiate when cultured by themselves in vitro. Cell-based therapies combining progenitor cells and primary cells can be used for repair and regeneration of damaged tissue and organs for treating bodily injuries and degenerative diseases.

The inventors have shown that adipose-derived stem cells and neonatal articular chondrocytes, co-encapsulated in mixed cultures in hydrogels comprising a PEG-based polymer such as PEGDA or PEGDMA and an extracellular matrix molecule such as chondroitin sulfate methacrylate (CS-MA), hyaluronic acid methacrylate (HA-MA), or heparan sulfate methacrylate (HS-MA), when cultured in the presence of TGF-β3, generated cartilage that could be used for treatment of traumatic injuries or diseases involving cartilage degeneration (see Examples 1-3). The hydrogel served as a three-dimensional scaffold controlling intercellular distance between the progenitor cells and primary cells. The hydrogel also retained released paracrine signaling molecules allowing paracrine signal distribution to the primary cells. Three co-culture models were tested: a mixed culture of primary cells and progenitor cells, a bilayered culture with primary cells and progenitor cells in separate layers, and a culture of primary cells with conditioned media from progenitor cells (Example 1). Of the three co-culture models tested, the mixed culture model provided the greatest degree of paracrine signal distribution to the primary cells, as well as intercellular contact between the primary cells and progenitor cells, and also the highest level of cartilage formation. Moreover, the inventors showed that progenitor cells could be used to stimulate cartilage formation with a minimal number of primary cells, as few as 1% or less, in mixed cultures containing primary cells and progenitor cells. Most unexpectedly, larger cartilage nodules formed as the number of chondrocytes was decreased in mixed cultures. Thus, a minimal number of primary cells in combination with progenitor cells can be used to achieve effective tissue repair.

In order to further an understanding of the invention, a more detailed discussion is provided below regarding cell-based therapies using progenitor cells in combination with tissue-specific primary cells.

The compositions for regenerating, replacing, or repairing tissue comprise a three-dimensional scaffold encapsulating progenitor cells and tissue-specific primary cells. The progenitor cells and tissue-specific primary cells can be combined in the scaffold as a mixed culture, in which the progenitor cells and primary cells are uniformly mixed. In the mixed culture, the ratio of the two cell types can be adjusted to achieve optimum production of the desired cell product. The three dimensional scaffold can be used to control the intercellular distance between the progenitor cells and primary cells and may bind and retain released paracrine signaling molecules allowing paracrine signal distribution to the primary cells.

Any three-dimensional scaffold that is biocompatible with the encapsulated cells and that allows production of the desired product from the primary cells may be used. Suitable biocompatible hydrogels for cell encapsulation are known and include, but are not limited to, hydrogels comprising polysaccharides, polyphosphazenes, poly(acrylic acids), poly(methacrylic acids), copolymers of acrylic acid and methacrylic acid, poly(alkylene oxides), poly(vinyl acetate), polyvinylpyrrolidone (PVP), and copolymers and blends of each. Polysaccharides that can be used include alginate, chitosan, hyaluronan, and chondroitin sulfate. See, e.g., Lee et al. (2008) Tissue Eng. Part A. 14(11):1843-1851; Hwang et al. (2007) Methods Mol. Biol. 407:351-373; Hwang et al. (2006) Stem Cells 24, 284-291; Lu et al. (2013) Int. J. Nanomedicine 8:337-350; Peng et al. (2012) Nanotechnology 23(48):485102; Pok et al. (2013) Acta Biomater. 9(3):5630-5642; Phadke et al. (2013) Eur. Cell Mater. 25:114-129; herein incorporated by reference.

In order to improve cell viability and tissue production, a biomimetic scaffold can be used that mimics certain aspects of the natural cell environment of the primary cell, such as the structure and function of the extracellular matrix (ECM). For example, a scaffold containing one or more ECM components can be used, such as a composite hydrogel scaffold containing at least one ECM component selected from the group consisting of a proteoglycan (e.g., chondroitin sulfate, heparan sulfate, and keratan sulfate), a non-proteoglycan polysaccharide (e.g., hyaluronic acid), a fiber (e.g., collagen and elastin), and any other ECM component (e.g., fibronectin and laminin). Preferably, the scaffold binds to one or more paracrine signaling molecules released from the encapsulated cells.

Chondroitin sulfate is one of the predominant structural proteoglycans in many tissues, including skin, cartilage, tendons, and heart valves and, therefore, is useful to include in biomimetic scaffolds for many tissue engineering applications. Hydrogels containing chondroitin sulfate can be prepared by modifying chondroitin sulfate with methacrylate groups followed by photopolymerization. The hydrogel properties can be readily controlled by the degree of methacrylate substitution and macromer concentration in solution prior to polymerization. Copolymer hydrogels of chondroitin sulfate and an inert polymer, such as polyethylene glycol (PEG) or polyvinyl alcohol (PVA) may also be used. See, e.g., Varghese et al. (2008) Matrix Biol. 27(1):12-21; Strehin et al. (2010) Biomaterials. 31(10):2788-2797; herein incorporated by reference.

The primary cells chosen for encapsulation depend on the desired therapeutic effect. The primary cells can be obtained directly from the patient to be treated, a donor, a culture of cells from a donor, or from established cell culture lines. Cells may be obtained from the same or a different species than the subject to be treated, but preferably are of the same species, and more preferably of the same immunological profile as the subject. Such cells can be obtained, for example, by biopsy from a close relative or matched donor.

The progenitor cells are chosen for their ability to promote tissue production from the primary cells. Progenitor cells that can be used include, but are not limited to, progenitor cells from various types of tissues, such as mesenchymal stromal cells from bone marrow, endothelial progenitor cells, muscle progenitor cells (e.g., satellite cells), pancreatic progenitor cells, periosteum progenitor cells, neural progenitor cells, blast cells, intermediate progenitor cells, and stem cells, including stem cells from embryos, umbilical cord, or adult tissues, or induced pluripotent stem cells.

The use of progenitor cells to catalyze tissue production by the primary cells allows a smaller number of primary cells to be used for tissue production than would be needed if the primary cells were used alone in tissue production. In one embodiment, the number of tissue-specific primary cells included in compositions used for tissue production is the minimal number needed to promote a therapeutically effective amount of tissue production to treat a particular injury or disease involving tissue degeneration. In one embodiment, the percentage of primary cells in a mixed culture with progenitor cells is 1% or less.

In certain embodiments, one or more additional factors, such as nutrients, cytokines, growth factors, antibiotics, anti-oxidants, or immunosuppressive agents may be added to the scaffold to improve cell function or viability. The composition may also further comprise a pharmaceutically acceptable carrier.

Exemplary growth factors include, fibroblast growth factor (FGF), insulin-like growth factor (IGF), transforming growth factor beta (TGF-β), epiregulin, epidermal growth factor (“EGF”), endothelial cell growth factor (“ECGF”), nerve growth factor (“NGF”), leukemia inhibitory factor (“LIF”), bone morphogenetic protein-4 (“BMP-4”), hepatocyte growth factor (“HGF”), vascular endothelial growth factor-A (“VEGF-A”), and cholecystokinin octapeptide.

Exemplary immunosuppressive agents are well known and may be steroidal (e.g., prednisone) or non-steroidal (e.g., sirolimus (Rapamune, Wyeth-Ayerst Canada), tacrolimus (Prograf, Fujisawa Canada), and anti-IL2R daclizumab (Zenapax, Roche Canada). Other immunosuppressant agents include 15-deoxyspergualin, cyclosporin, methotrexate, rapamycin, Rapamune (sirolimus/rapamycin), FK506, or Lisofylline (LSF).

One or more pharmaceutically acceptable excipients may also be included. Exemplary excipients include, without limitation, carbohydrates, inorganic salts, antimicrobial agents, antioxidants, surfactants, buffers, acids, bases, and combinations thereof.

For example, an antimicrobial agent for preventing or deterring microbial growth may be included. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof. Antibmicrobial agents also include antibiotics that can also be used to prevent bacterial infection. Exemplary antibiotics include amoxicillin, penicillin, sulfa drugs, cephalosporins, erythromycin, streptomycin, gentamicin, tetracycline, chlarithromycin, ciproflozacin, azithromycin, and the like. Also included are antifungal agents such as myconazole and terconazole.

Various antioxidants can also be included, such as molecules having thiol groups such as reduced glutathione (GSH) or its precursors, glutathione or glutathione analogs, glutathione monoester, and N-acetylcysteine. Other suitable anti-oxidants include superoxide dismutase, catalase, vitamin E, Trolox, lipoic acid, lazaroids, butylated hvdroxyanisole (BHA), vitamin K, and the like.

Excipients suitable for injectable compositions include water, alcohols, polyols, glycerin, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

Acids or bases can also be present as an excipient. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D. C., 2000.

In one embodiment, the invention includes a composition for generating new cartilage comprising adipose-derived stem cells and chondrocytes encapsulated in a hydrogel. In certain embodiments, the adipose-derived stem cells and chondrocytes are combined in a mixed culture in the hydrogel, wherein the ratio of adipose-derived stem cells to chondrocytes is about 25:75, about 50:50, about 75:25, about 90:10, about 95:5, about 98:2, about 99:1, or any ratio in between. In another embodiment, the percentage of cells in the mixed culture that are chondrocytes is 1%-2%, 2%-5%, 5%-10%, 10-25%, or any percentage within these ranges, including 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%. In another embodiment, the percentage of chondrocytes in the mixed culture is 1% or less. In another embodiment, the number of chondrocytes is the minimal number needed to promote a therapeutically effective amount of cartilage production to treat an injury or disease involving cartilage degeneration.

The hydrogel composition may comprise a polyethylene glycol (PEG)-based hydrogel. Exemplary PEG-based hydrogels include poly(ethylene glycol) diacrylate (PEGDA) and poly(ethylene glycol) dimethacrylate (PEGDMA) hydrogels. The hydrogel composition may also comprise at least one extracellular matrix molecule, including, but not limited to, chondroitin sulfate methacrylate (CS-MA), hyaluronic acid methacrylate (HA-MA), and heparan sulfate methacrylate (HS-MA). In certain embodiments, at least one extracellular matrix molecule is present in the hydrogel at a concentration ranging from about 0.5% (w/v) to about 5% (w/v), or any concentration within this range, including 0.5, 0.75, 1.0, 1.25, 1.5, 1.75, 2.0, 2.25, 2.5, 2.75, 3.0, 3.25, 3.5, 3.75, 4.0, 4.25, 4.5, 4.75, or 5.0% (w/v). In certain embodiments, the hydrogel composition comprises PEGDMA at a concentration ranging from about 8% (w/v) to about 14% (w/v) or any concentration within this range, including 8, 8.5, 9, 9.5, 10, 10.5, 11, 11.5, 12, 12.5, 13, 13.5, or 14% (w/v). In certain embodiments, the hydrogel has a mechanical stiffness having a Young's modulus of from about 3 kPa to about 100 kPa or any value within this range, including 3, 4, 5, 6, 7, 8, 9, 10, 11, 12, 13, 14, 15, 20, 25, 30, 35, 40, 45, 50, 55, 60, 65, 70, 75, 80, 85, 90, 95, or 100 kPa.

The compositions, described herein, for transplanting cells are typically, though not necessarily, administered by injection or surgical implantation into a region requiring tissue replacement or repair. For example, compositions capable of producing new cartilage in a subject can be administered locally into an area of cartilage damage or loss, such as a damaged joint or other suitable treatment site of the subject.

In another embodiment, the invention includes a method for treating a subject for tissue damage or loss comprising administering a therapeutically effective amount of a composition comprising progenitor cells and tissue-specific primary cells, encapsulated in a three-dimensional scaffold, to the subject. By “therapeutically effective dose or amount” of a composition comprising progenitor cells and tissue-specific primary cells is intended an amount that, when administered as described herein, brings about a positive therapeutic response in a subject having tissue damage or loss, such as an amount that results in the generation of new tissue at a treatment site.

For example, a therapeutically effective dose or amount of a composition comprising adipose-derived stem cells and chondrocytes is intended an amount that, when administered as described herein, brings about a positive therapeutic response in a subject having cartilage damage or loss, such as an amount that results in the generation of new cartilage at a treatment site (e.g., a damaged joint). For example, a therapeutically effective dose or amount could be used to treat cartilage damage or loss resulting from a traumatic injury or a degenerative disease, such as arthritis or other disease involving cartilage degeneration. Preferably, a therapeutically effective amount restores function and/or relieves pain and inflammation associated with cartilage damage or loss.

In certain embodiments, the invention includes a method of treating a subject for cartilage damage or loss, the method comprising administering a therapeutically effective amount of a composition, described herein, comprising adipose-derived stem cells and chondrocytes to the subject. The chondrocytes used in treatment may be autologous or allogeneic. Preferably, the chondrocytes are derived from the patient or a matched donor. After transplantation of the hydrogel composition comprising the mixed culture to the patient, the chondrocytes in the hydrogel composition produce new cartilage in vivo. Such cartilage is capable of filling cartilage defects of any shape and size at the treatment site. The new cartilage can be produced in vivo even under hypoxic conditions, for example, wherein the local O₂ tension ranges from 1% to 7%.

In another embodiment, the invention includes a method for treating a patient for cartilage damage or loss, the method comprising: a) combining chondrocytes with adipose-derived stem cells in a mixed culture, wherein the mixed culture comprises 1% to 25% chondrocytes and 75% to 99% adipose-derived stem cells; b) adding the mixed culture to a hydrogel composition comprising chondrogenic media, TGF-β3, and at least one extracellular matrix molecule selected from the group consisting of chondroitin sulfate methacrylate (CS-MA), hyaluronic acid methacrylate (HA-MA), and heparan sulfate methacrylate (HS-MA), wherein the hydrogel composition has a Young's modulus of from about 3 kPa to about 100 kPa; and c) transplanting the hydrogel composition comprising the mixed culture to the patient at a site in need of cartilage replacement. In another embodiment, the method further comprises administering an effective amount of TGF-β3 to the patient after transplantation of the hydrogel to the patient.

In another embodiment, the hydrogel composition comprising the mixed culture is transplanted to the patient after culturing the chondrocytes ex vivo in the hydrogel composition for a period of time. For example, the chondrocytes can be cultured in a mixed culture with the adipose-derived stem cells for a few days or weeks, such as at least 1, day, 2 days, 3 days, 4 days, 5 days, 6 days, 1 week, 1.5 weeks, 2 weeks, 2.5 weeks, 3 weeks, or longer prior to transplantation of the hydrogel composition to the patient.

In another embodiment, the invention includes a method for producing cartilage, the method comprising: a) obtaining chondrocytes from a subject; b) combining the chondrocytes with adipose-derived stem cells in a mixed culture, wherein the mixed culture comprises 1% to 25% chondrocytes and 75% to 99% adipose-derived stem cells; c) adding the mixed culture to a hydrogel composition, wherein the hydrogel composition has a Young's modulus of from about 3 kPa to about 100 kPa; culturing the chondrocytes ex vivo or in vivo in the hydrogel composition, wherein the chondrocytes are cultured in the mixed culture with the adipose-derived stem cells in chondrogenic media comprising TGF-β3 and at least one extracellular matrix molecule selected from the group consisting of chondroitin sulfate methacrylate (CS-MA), hyaluronic acid methacrylate (HA-MA), and heparan sulfate methacrylate (HS-MA) under conditions, whereby cartilage is produced comprising nodules having a nodule size of at least 100 μm in length.

In another embodiment, the invention includes a method of treating a patient for cartilage damage or loss, the method comprising producing cartilage by a method described herein, and transplanting the cartilage to the patient at a site in need thereof. The cartilage may be administered, for example, locally at a damaged joint of the subject to treat a subject having a traumatic injury or a disease involving cartilage degeneration (e.g., arthritis).

Any of the compositions described herein may be included in a kit. The kit may comprise one or more containers holding the implant comprising the three-dimensional scaffold containing the encapsulated primary cells and progenitor cells or primary cells and conditioned media from progenitor cells. Alternatively, the kit may comprise the individual components needed for preparing an implant, such as the reagents for generating the three-dimensional scaffold, progenitor cells, primary cells, media, and optionally one or more other factors, such as growth factors, ECM components, antibiotics, and the like). Suitable containers for the compositions include, for example, bottles, vials, syringes, and test tubes. Containers can be formed from a variety of materials, including glass or plastic. A container may have a sterile access port (for example, the container may be a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

The kit can also comprise a package insert containing written instructions for methods of treating tissue damage or loss, such as caused by a traumatic injury or a disease involving tissue degeneration. The package insert can be an unapproved draft package insert or can be a package insert approved by the Food and Drug Administration (FDA) or other regulatory body.

In certain embodiments, the kit comprises a hydrogel composition for generating new cartilage, as described herein, or reagents and cells for preparing such a composition (e.g., TGF-β3, CS-MA, HA-MA, HS-MA, PEGDA, and/or PEGDMA, adipose-derived stem cells, chondrocytes, media, and optionally one or more other factors, such as other growth factors or ECM components, antibiotics, and the like). The kit may also comprise means for delivering the composition to a subject and instructions for treating a traumatic injury or a disease involving cartilage degeneration.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Adipose-Derived Stem Cells Catalyze Cartilage Formation by Neonatal Articular Chondrocytes

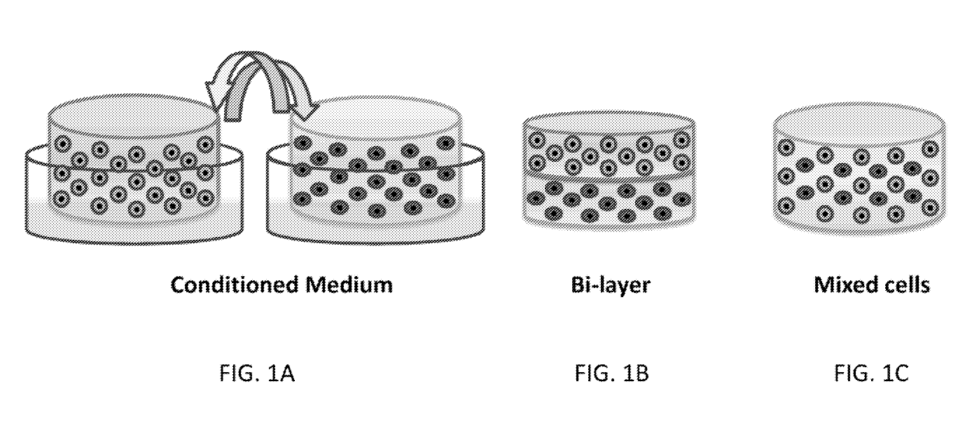

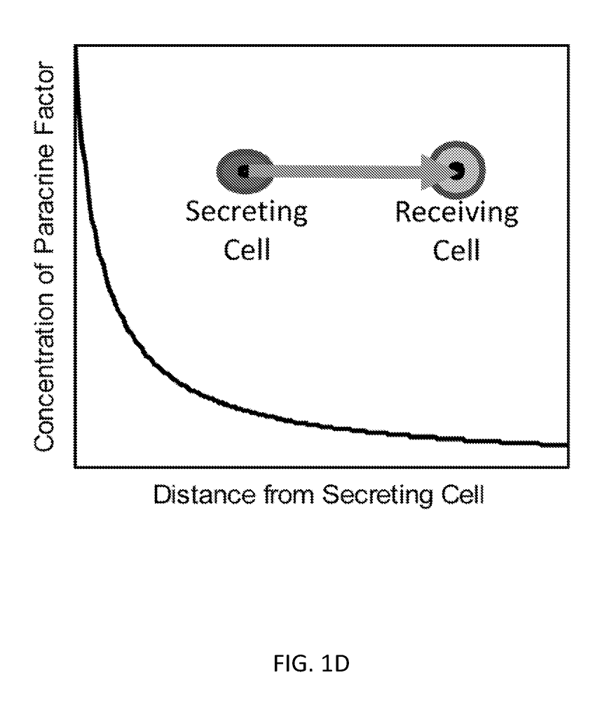

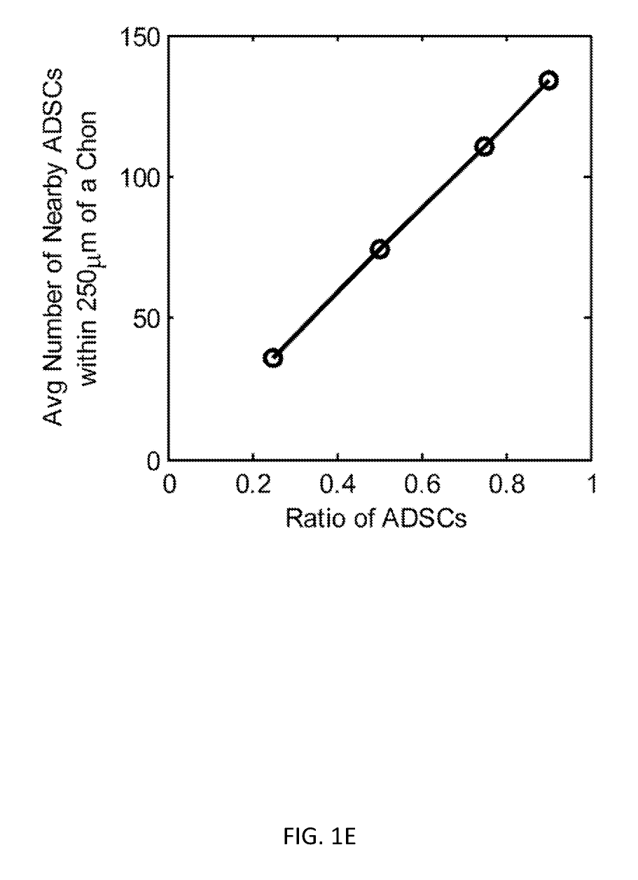

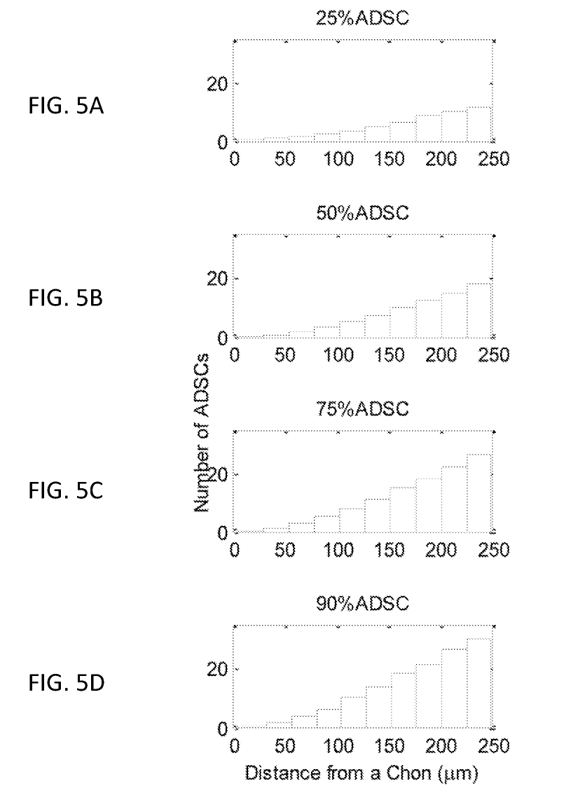

Here we report the use of adipose-derived stem cells (ADSCS) to catalyze cartilage formation by neonatal articular chondrocytes (CHONS) for cartilage regeneration. In order to examine the interaction between ADSCS and CHONS, three different in vitro culture models were used: 1) cells cultured with supplementation of conditioned medium from the other cell type (FIG. 1A), 2) bi-layered culture that confines the two cell types in separate layers mixed co-culture at different cell ratios (FIG. 1B), and 3) mixed culture of the two cells at different cell ratios (FIG. 1C). These culture models are designed specifically to allow for cells to interact at different levels of proximity, which is a governing parameter in cell-cell communication. The concentration of paracrine factors secreted by a cell decays exponentially with distance from the secreting cell (FIG. 1D), and the effective communication distance over which a cell can propagate soluble signal is within 250 μm (Francis and Palsson (1997) Proc. Natl. Acad. Sci. U.S.A. 94:12258-1262). In the mixed cell culture model, increasing ADSC ratio while keeping the overall cell density constant leads to a linear increase in the number of ADSCS that are within the effective communication distance of a CHON (FIG. 1E and FIG. 5).

In all the culture models, cells were encapsulated in a 3D biomimetic hydrogel consisting of chondroitin sulfate methacrylate (CS-MA) and poly(ethylene) glycol diacrylate (PEGDA), which enables enzymatic degradation and matrix turnover by cell-secreted chondroitinase (Varghese et al. (2008) Matrix Biol. 27:12-21; herein incorporated by reference). Human adult ADSCS and bovine neonatal CHONS were encapsulated in 3D biomimetic hydrogels and cultured in vitro for 21 days in chondrogenic medium supplemented with TGF-β3.

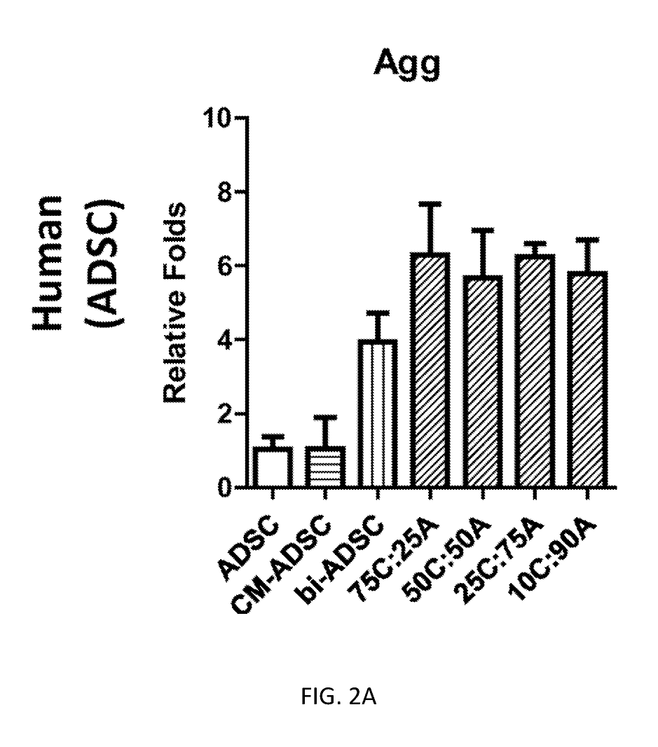

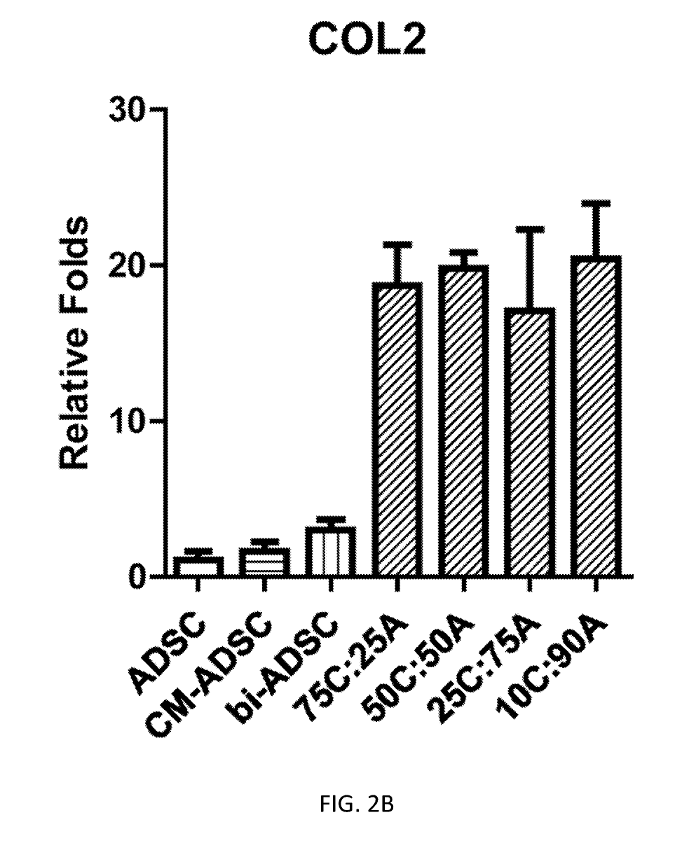

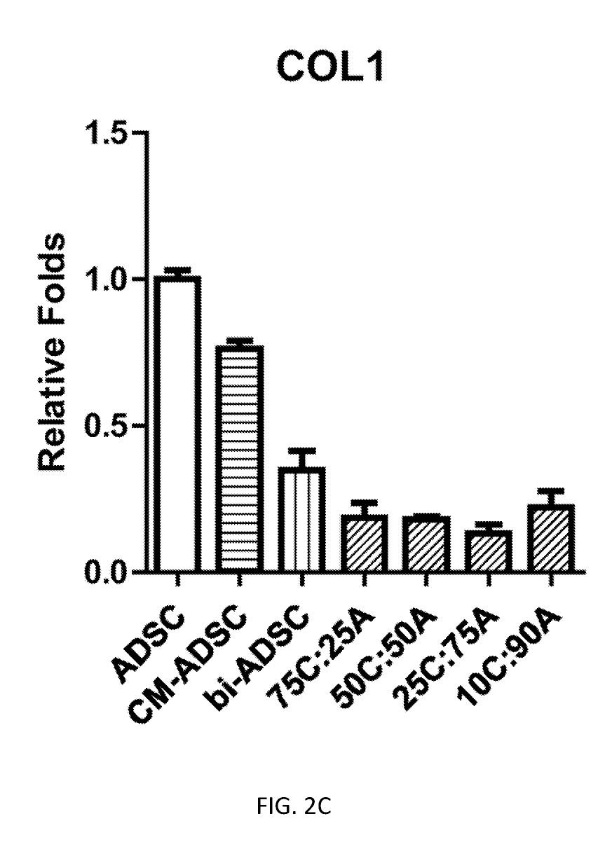

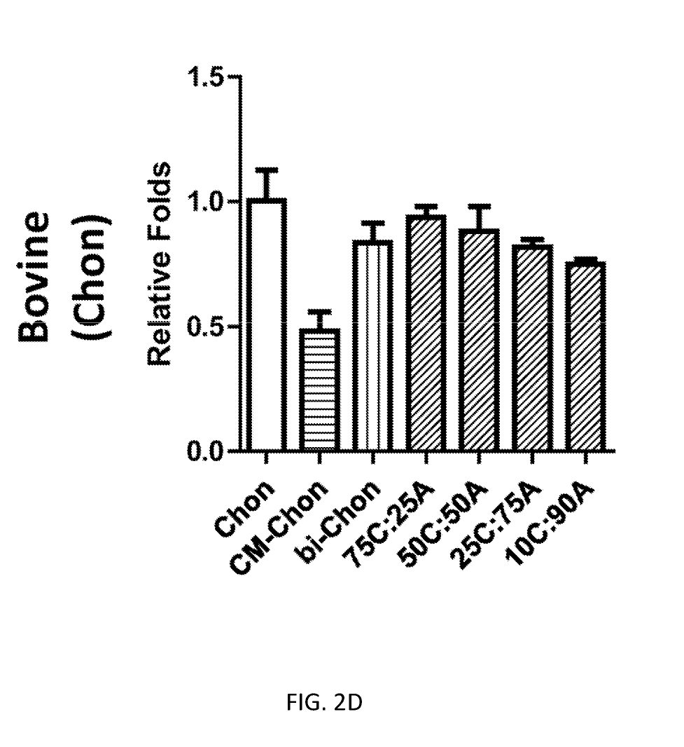

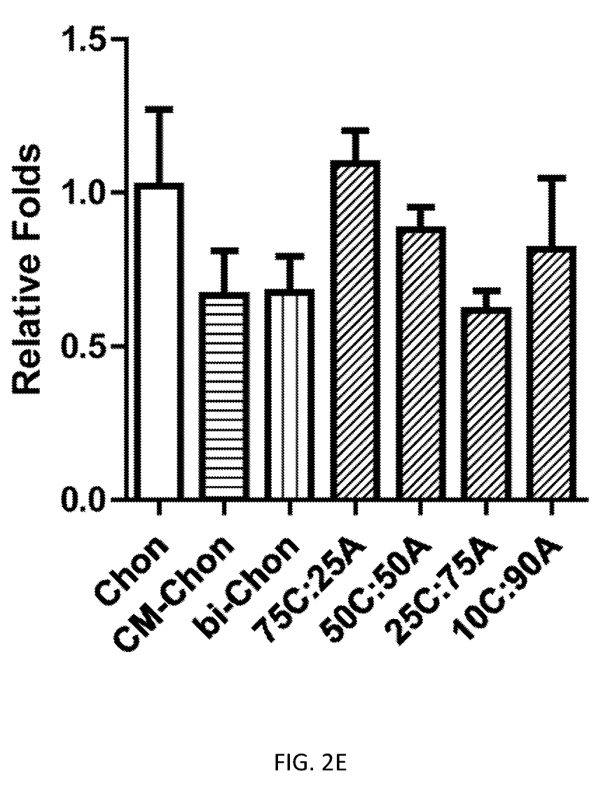

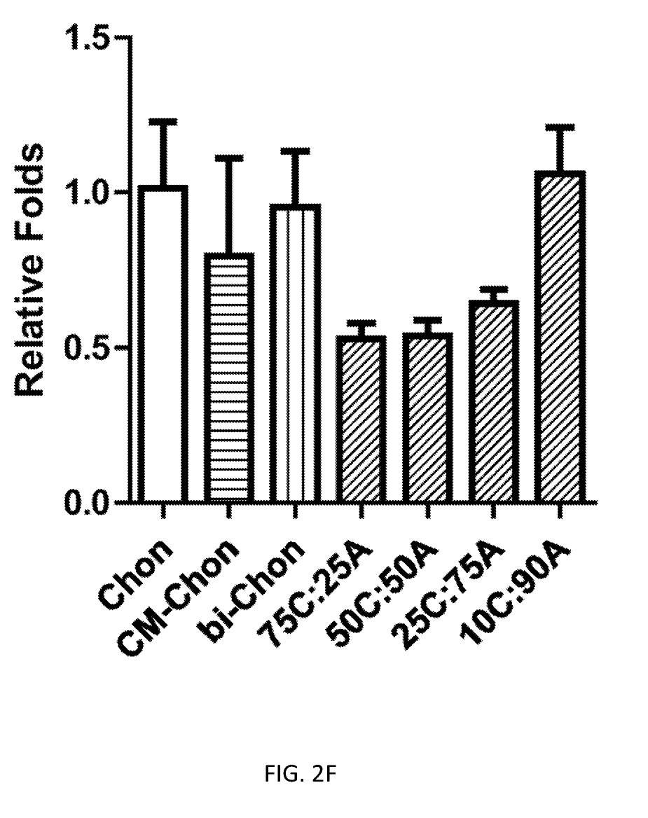

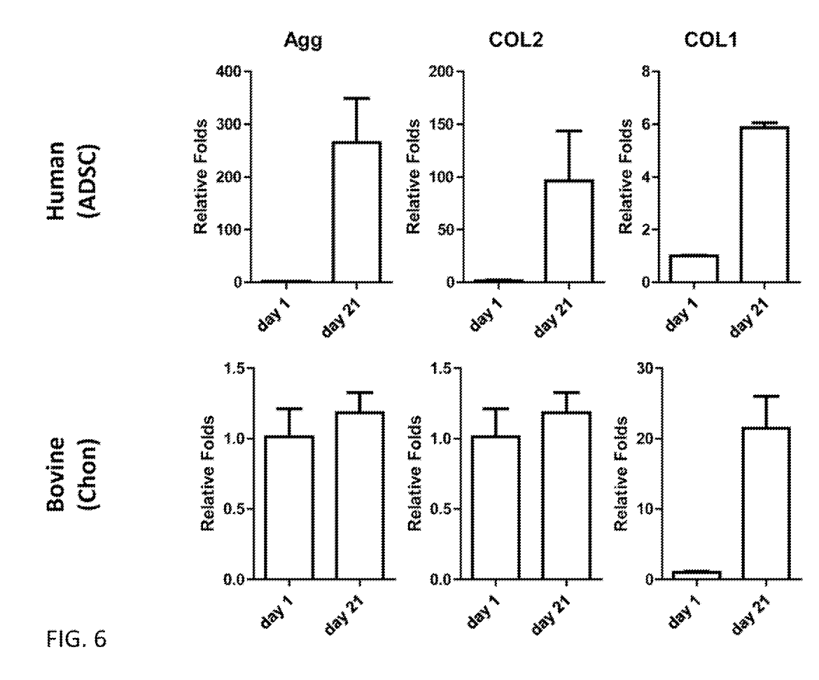

Gene markers associated with chondrocytes were evaluated with reverse transcriptase polymerase chain reaction (RT-PCR). To distinguish the fate of each cell type, specie-specific primers were used. At day 21, gene expression of aggrecan (Agg) and type II collagen (COL2) of ADSCS increased 265- and 96-fold respectively (FIGS. 6A and 6B), indicating that ADSCS underwent chondrogenesis under the induction of TGF-β3. ADSCS treated with conditioned medium collected from CHONS (CM-ADSC) resulted in an increase in chondrogenic gene expression (Agg and COL2) and a decrease in the fibroblastic marker, type I collagen (COL1) compared to the ADSC control (FIG. 2C). When ADSCS were cultured with CHONS in a bi-layered hydrogel (bi-ADSC), which allowed for a higher concentration as well as a dynamic exchange of paracrine factor compared to CM-ADSC, chondrogenic expression (Agg and COL2) was further increased while fibroblastic expression (COL1) was further decreased. As for CHONS, both treatment with conditioned medium collected from ADSCS (CM-CHON) and the bi-layer culture (bi-CHON) did not lead to significant changes in Agg, COL2, and COL1 expression (FIGS. 2D, 2E, and 2F).

Mixed cell culture, which allows for the two types to interact at close proximity, led to a marked increase in chondrogenic expression in ADSCS, maintenance of chondrocyte phenotype in CHONS, and reduction of fibroblastic expression in both cell types. Interestingly, changing cell ratios in the mixed co-culture did not lead to significant changes in chondrogenic gene expression. Agg and COL2 expression in the mixed co-culture groups exhibited 1400-1600 and 1500-1800 fold increases, respectively, over 21 days, which were 5.5-6 and 15.9-19 times higher than those of the ADSC control at day 21 (FIGS. 2A and 2B). COL1 expression was reduced compared to the ADSC control (FIG. 2C). As for CHONS, chondrogenic gene expression in mixed co-culture groups was maintained at levels similar to the CHON control (FIGS. 2D and 2E). Agg expression of CHONS in all the mixed co-culture groups except for 10C:90A was comparable to that of the CHON control. Similarly, COL2 expression of CHONS in the mixed co-culture groups was comparable to that of the CHON control. COL2 expression of the 25C:75A group was significantly lower than that of 75C:25A. COL1 expression in all the mixed co-culture groups except 10C:90A was reduced to approximately 50% of that of the CHON control (FIG. 2F).

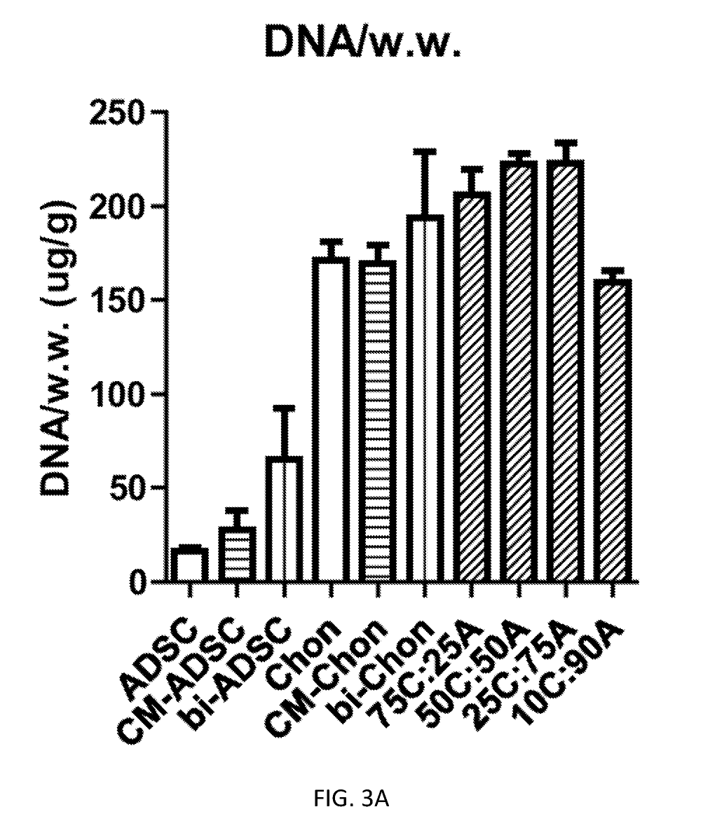

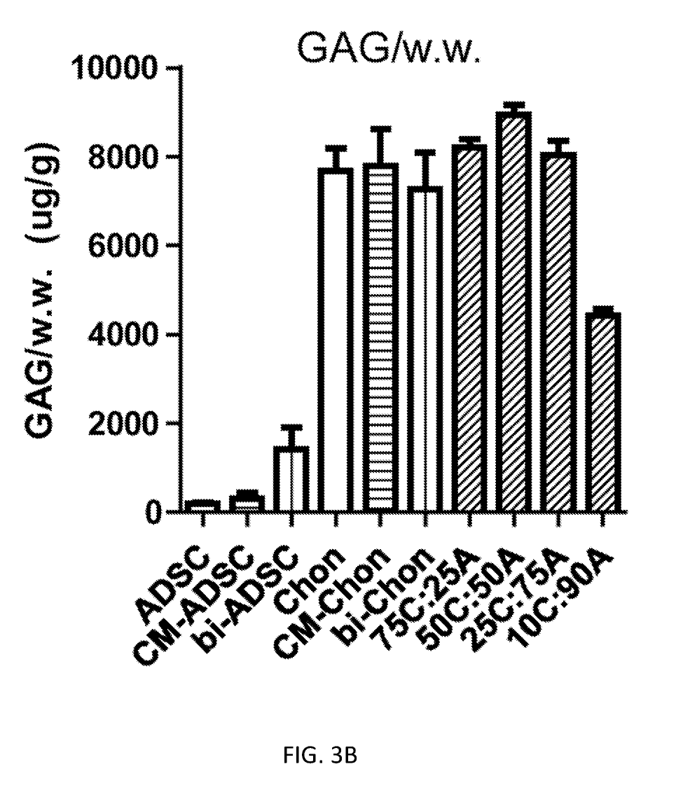

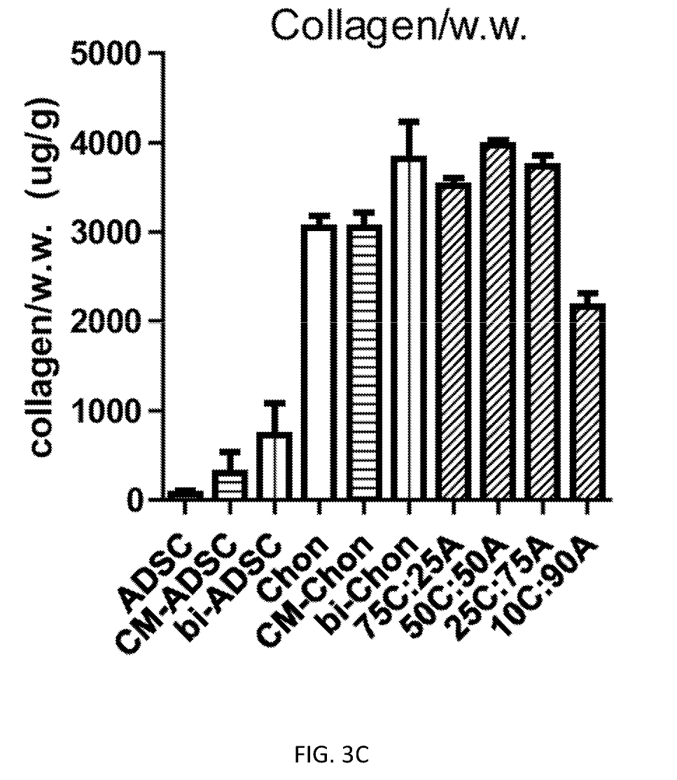

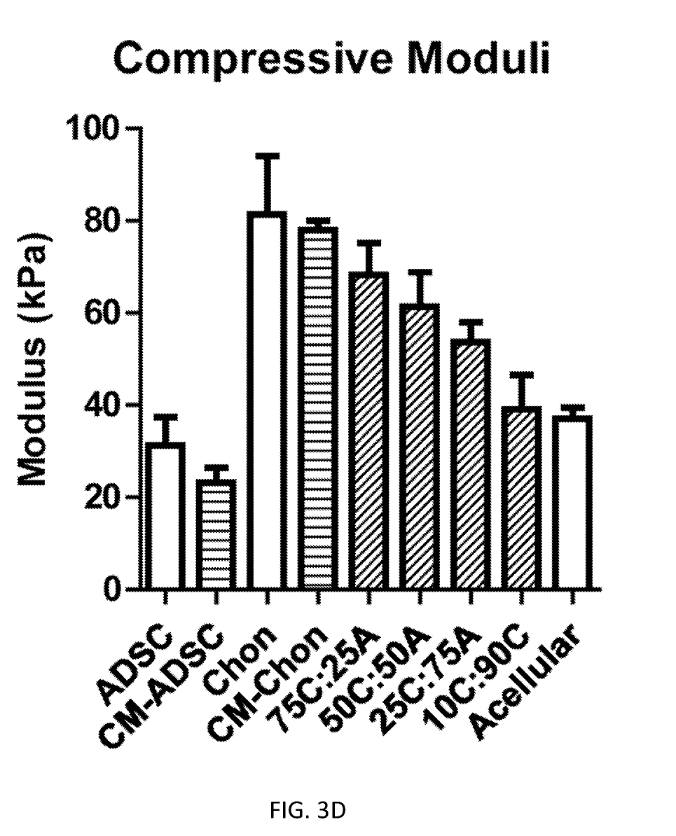

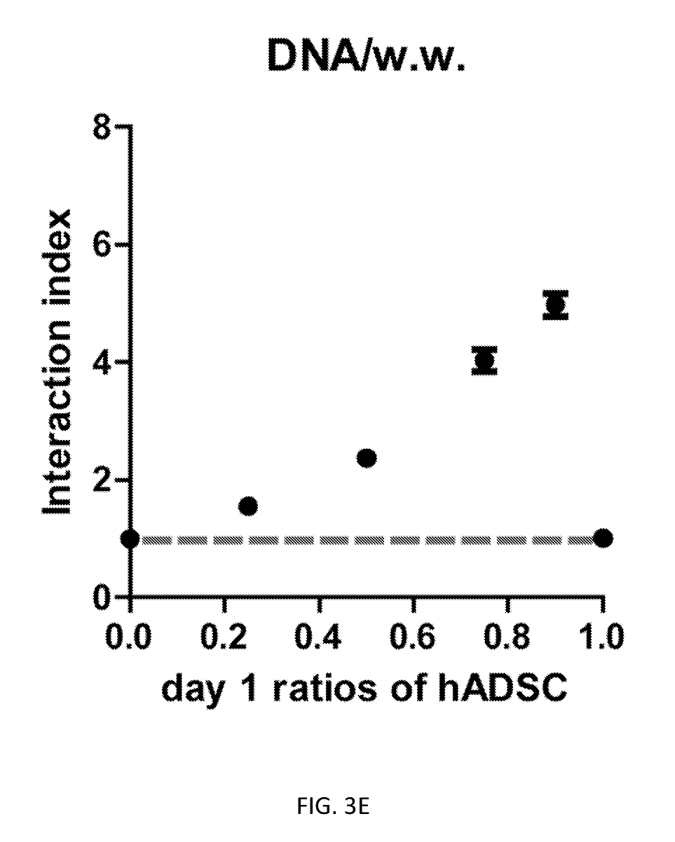

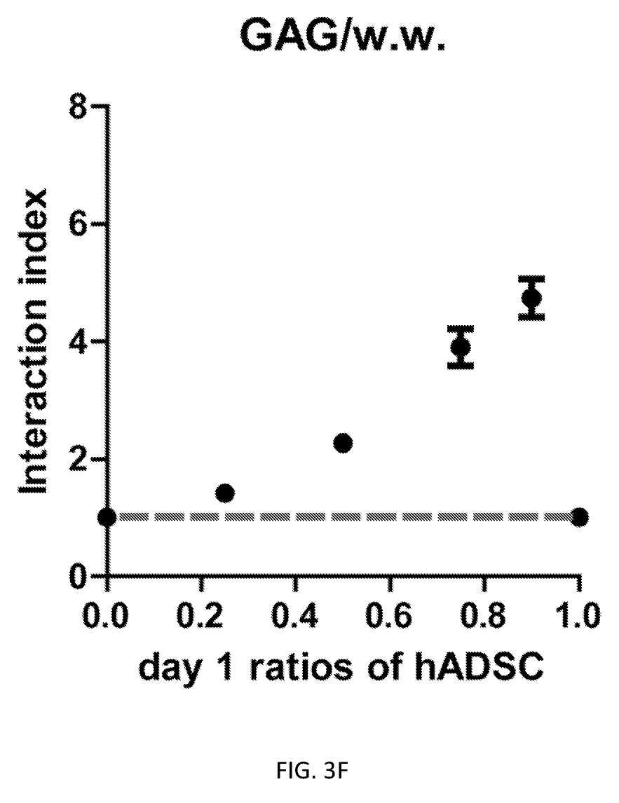

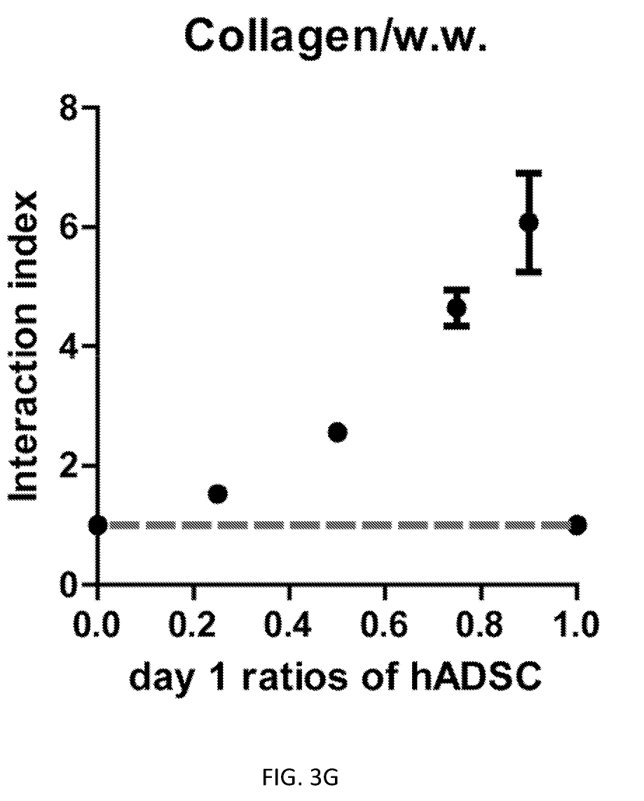

Next, we quantified cell proliferation and cartilage matrix production. To quantify cell proliferation over time, DNA contents were measured at day 1 and 21. In the ADSC control group, DNA content at day 21 was reduced to 29% of day 1 DNA content (FIG. 3A). Both conditioned medium and bi-layer co-culture had significantly higher numbers of ADSCS than that of the ADSC control at day 21. To quantify cartilage matrix production, sulfated glycosaminoglycan (sGAG) and total collagen content were measured at day 21. SGAG and collagen per wet weight exhibited similar trends (FIGS. 3B and 3C). ADSC cultured in conditioned medium led to approximately 1.6- and 3-fold increases in sGAG and collagen per wet weight respectively compared to the ADSC control group. The effect of bi-layer culture on ADSC matrix production was more significant, resulting in 7.6 and 10.4 fold increases in sGAG and collagen per wet weight respectively. As for CHONS, cell number and sGAG content per wet weight were maintained in conditioned medium and bi-layer culture (FIGS. 3A and 3B). Collagen content per wet weight in the conditioned medium group was similar to that of the CHON control group, but was significantly increased in the bi-layer group (bi-CHON) (FIG. 3C).

Mixed cell culture led to significantly higher cell number and cartilage matrix content than ADSC control. Gross appearance of the freeze-dried cell-hydrogel constructs indicated that matrix was formed in all the mixed co-culture groups and the CHON control group after 21 days of culture (FIG. 6), while the freeze-dried construct of the ADSC control group remained similar in size overtime. In all the mixed cell groups and CHON control group, DNA per wet weight increased significantly (about 3.1 to 4.2-fold) over 21 days of culture (FIG. 3A). The variations in sGAG and collagen content per wet weight with changes in cell ratios exhibited similar trends (FIGS. 3B and 3C). Of all the cell ratios examined, the 50C:50A group resulted in the most cartilage matrix formation, reaching up to 30% higher sGAG and collagen content per wet weight than the CHON control group. Surprisingly, mixed cell culture with as low as 25% CHONS (25C:75A) resulted in higher sGAG (˜18%) and collagen (about 22%) per wet weight than CHONS alone. When the percentage of CHONS in mixed co-culture was further reduced (10C:90A), sGAG and collagen content per wet weight dropped significantly to approximately 59 and 71% of CHON control group respectively. Elastic modulus in CHON control and all the mixed co-culture groups increased significantly over 21 days (FIG. 3D). At day 21, the modulus was the highest in the CHON control group and decreased progressively with an increase in ADSC ratio in the co-culture population.

To quantify the effects of cell ratio variation on cell proliferation and cartilage matrix production, the measured DNA, sGAG, and collagen content were compared against the expected values. At each cell ratio, the interaction index, which is the measured matrix content (DNA, sGAG, or collagen) in the mixed co-culture group normalized by the expected matrix content based on the measured matrix content in the CHON and hADSC alone groups, was calculated as previously shown by Acharya et al. (Acharya et al. (2012) J. Cell Physiol. 227:88-97; herein incorporated by reference). The interaction for DNA, sGAG, and collagen per wet weight in all the mixed co-culture groups were higher than 1 (FIGS. 3E, 3F, and 3G). Interestingly, the interaction index increased with an increase ratio of ADSCS in the mixed co-culture population, indicating that the extent of cell proliferation and cartilage matrix production was highly dependent on the ratio of the two cell types. At 90% ADSC (10C:90A), DNA, sGAG, and collagen content per wet weight were approximately 5-6-fold higher than expected. When normalized by DNA, however, the interaction index for collagen and sGAG was close to 1 (not shown), indicating that sGAG and collagen production were not increased significantly on a per cell basis. This suggests that mixed cell culture enhanced cartilage production primarily through the stimulation of cell proliferation. Remarkably, although an inverse relationship between cell proliferation and matrix production per cell has been reported in the literature (Detamore and Athanasiou (2004) Arch. Oral Biol. 49:577-583), in our study the increased cell proliferation as a result of mixed cell culture did not reduce matrix production on a per cell basis.

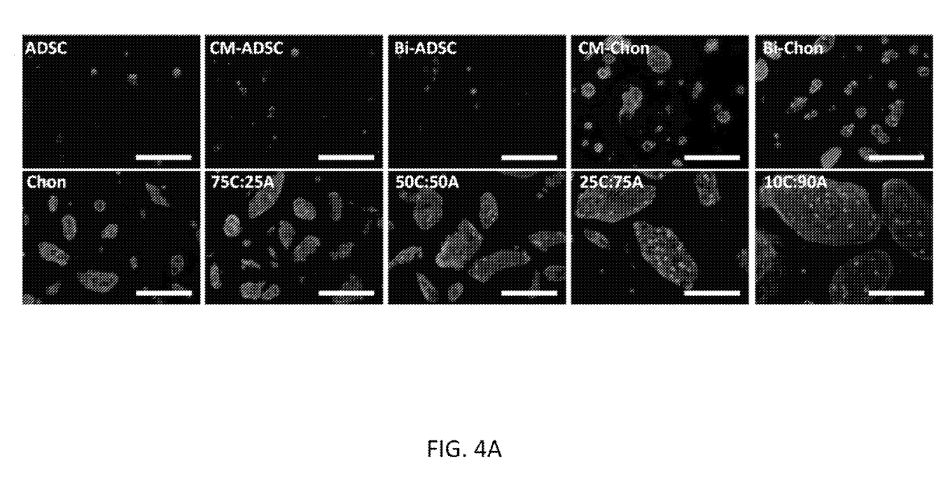

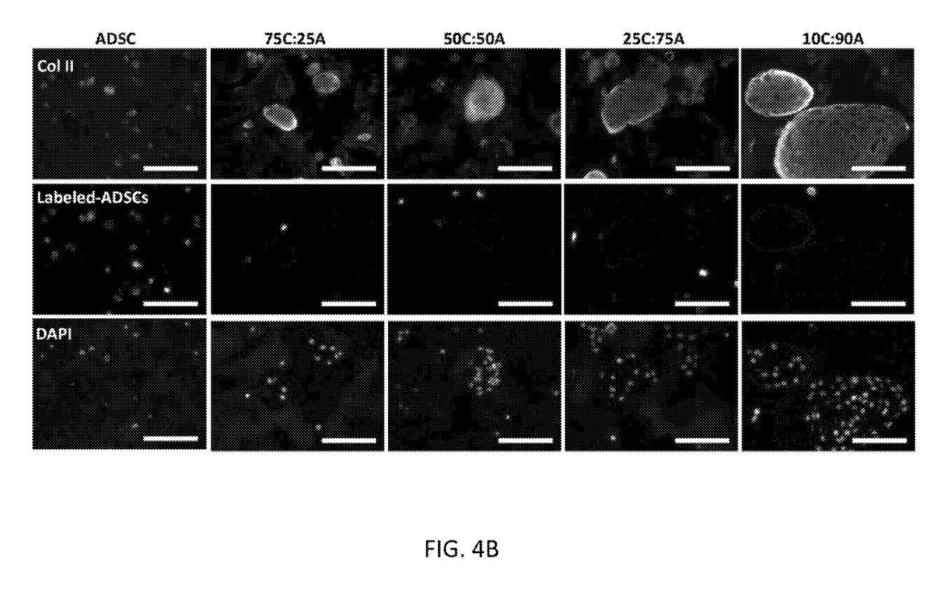

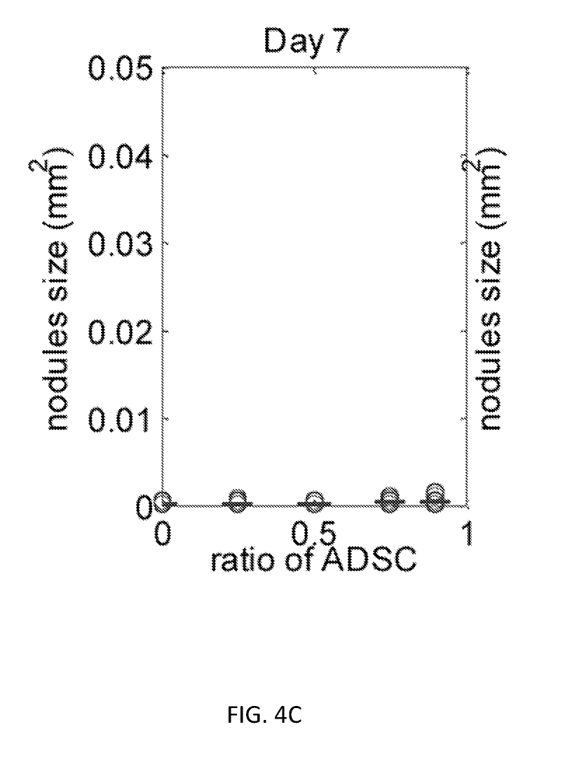

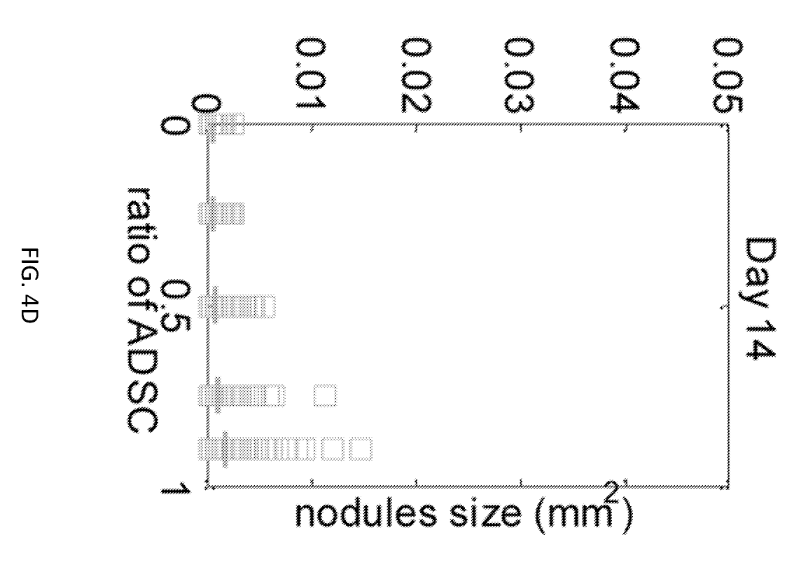

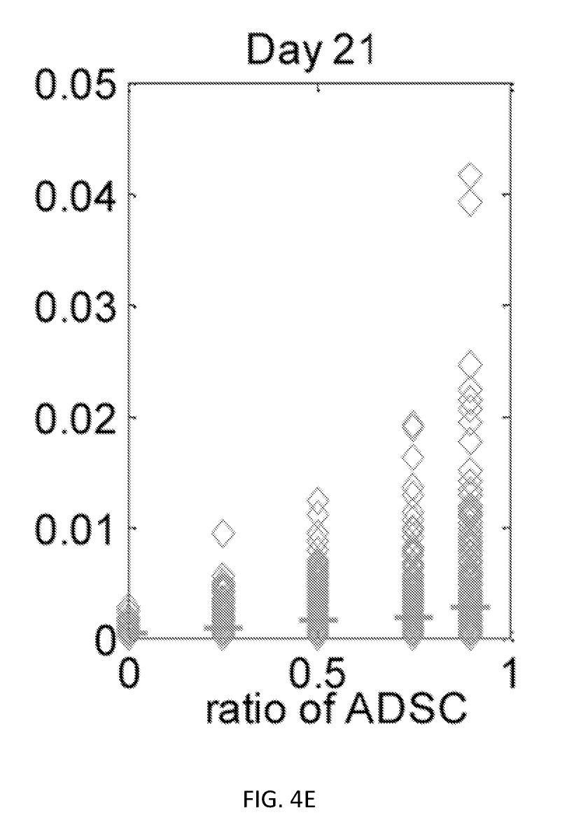

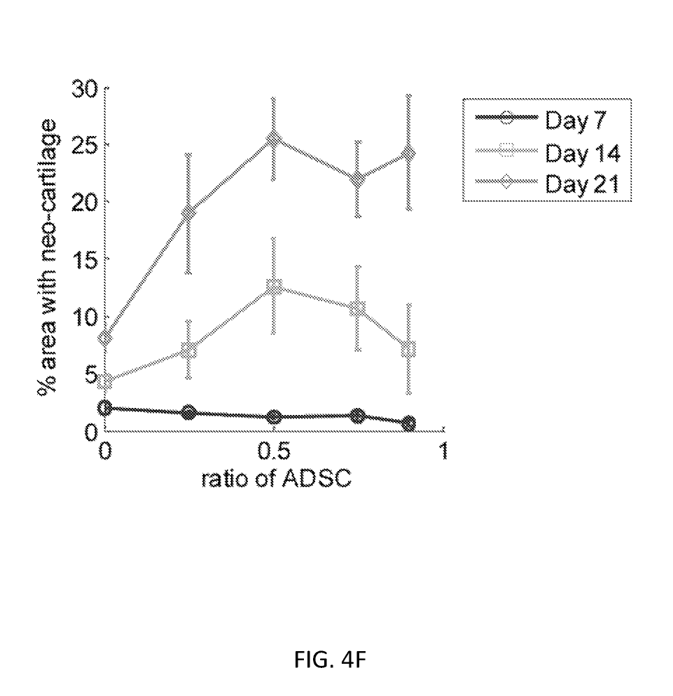

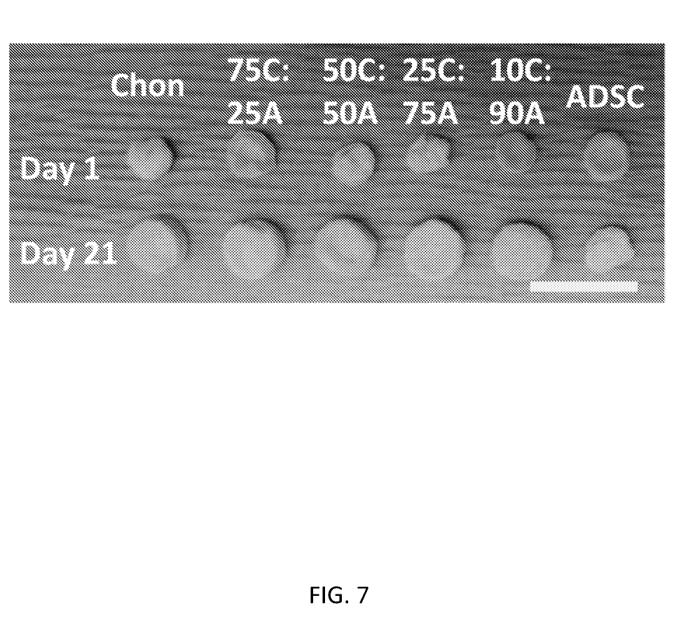

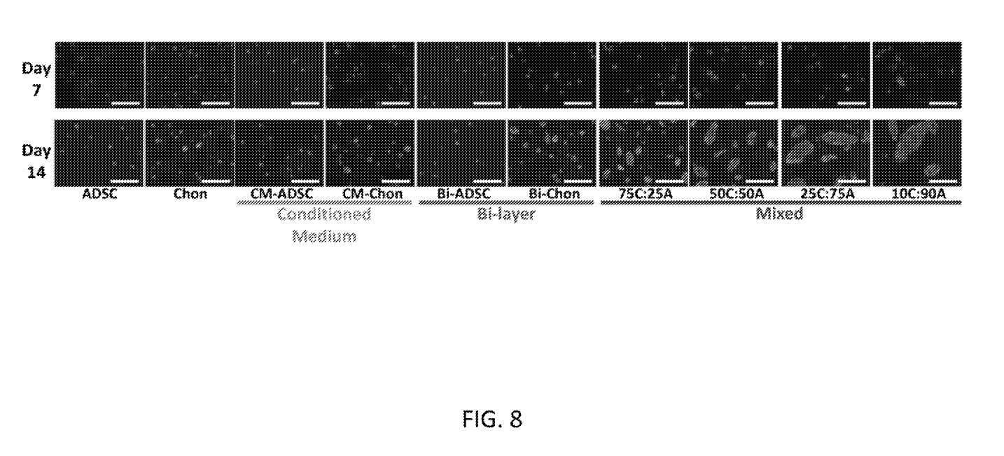

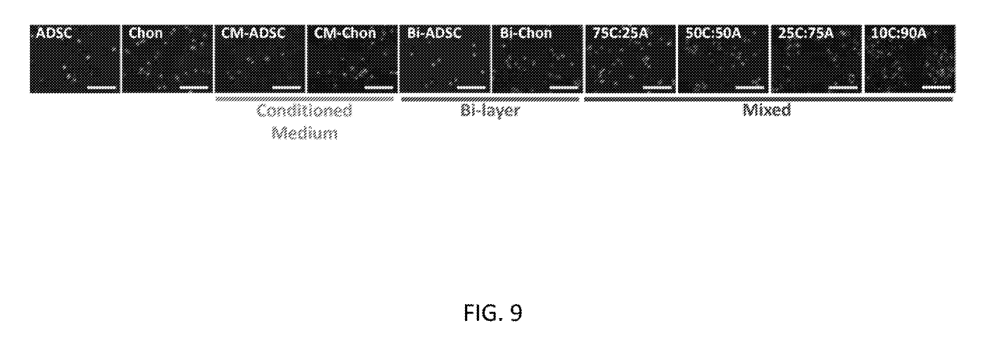

In addition to the extent of cell proliferation and cartilage matrix production, the differential effects of conditioned medium, bi-layer, and mixed cell culture on cartilage matrix production were also evident in the spatial organization of neo-cartilage within the 3D hydrogels as shown by type II collagen immunostaining. Conditioned medium and bi-layer culture did not lead to obvious changes in type II collagen production for both cell types. On the contrary, variation in cell ratios in the mixed cell culture led to differential formation and spatial organization of neo-cartilage nodules within the 3D hydrogels. While cells appeared to distribute evenly in the hydrogel matrix at day 7 (FIG. 7), cell aggregates and neo-cartilage nodules were observed at day 14 (FIG. 7) and 21 (FIG. 4A). Interestingly, the individual nodule size as well as the total area occupied by the nodules increased with an increase in ADSC ratio (FIGS. 4C-4E); at day 21, the nodule size in the group with 90% ADSCS (10C:90A) was 6 times larger than that in bAC alone group. It is also worth noting that while the mixed co-culture group with 90% hADSCS (10C:90A) was remodeled extensively with large neo-cartilage nodules, the control group with 100% ADSCS exhibited little cartilage deposition (FIG. 4A). Type I collagen staining was minimal (FIG. 9) in all groups, indicating that the cartilage nodules produced were hyaline cartilage instead of fibrocartilage. The drastic differences in neo-cartilage organization in the mixed cell culture demonstrated the cell fate as well as tissue formation was tightly regulated by the dynamic cell-cell interactions between CHONS and ADSCS. Using a 3D biomimetic hydrogel culture system enable us to observe the spatial and temporal differences in hydrogel remodeling and cartilage matrix organization at different cell ratios.

To further examine the distribution and the relative contribution of each cell type to cartilage nodule formation in the mixed co-culture, ADSCS were fluorescently labeled with lipophilic membrane dyes prior to encapsulation in hydrogels. Fluorescently labeled ADSCS were distributed throughout the hydrogel matrix at different time points, while aggregates of CHONS (negatively labeled cells) were observed on day 14 and 21. Direct cell-cell contact between the two cell types was not evident. Cell tracking along with co-localization of collagen II immunostaining in mixed cell culture indicated that cartilage nodules were formed primarily by aggregates of CHONS (FIG. 4B). This is in agreement with recent studies that showed that BMSCs stimulated CHON proliferation and cartilage matrix production (Acharya et al., supra; Wu et al. (2012) Tissue Eng. Part A. 18:1542-1551; Meretoja et al. (2012) Biomaterials 33:6362-6369). The lack of ADSCS within the cartilage nodules in mixed co-culture hydrogels, together with the increase in the size of these nodules with an increase in ADSC ratio in the mixed co-culture, suggested that the two cell types interact through paracrine signaling in a dose-dependent manner. With an increase in ADSCS in the mixed co-culture system, the number of ADSCS within effective communication distance of the CHONS increased, which stimulated CHONS to proliferate and produce larger cartilage nodules. It has been shown that ADSCS secrete growth factors such as fibroblast growth factor-2 (FGF-2), vascular endothelial growth factor (VEGF), hepatocyte growth factor (HGF), insulin-like growth factor 1 (IGF-1), which are known to stimulate cell proliferation. Of these factors, FGF-2 and IGF-1 have been shown to induce GAG and type II collagen synthesis in chondrocytes (Veilleux et al. (2005) Osteoarthritis Cartilage 13:278-286). In addition, FGF-2 has also been shown to reduce fibroblastic and hypertrophic phenotype in chondrocytes (Kato et al. (1990) J. Biol. Chem. 265:5903-5909; Martin et al. (2001) J. Cell Biochem. 83:121-128). It has been shown that the differentiation state of stem cells may impact their role as a stimulator for tissue formation. For instance, Rothenberg et al. showed that BMSCs that were pre-differentiated towards osteogenic lineage for 3 days acted as a stronger stimulator for cartilage tissue formation when co-cultured with chondrocytes than naïve BMSCs (Rothenberg et al. (2011) Stem Cells Dev. 20:405-414). In our culture system, ADSCS differentiated towards chondrogenic lineage under the induction of TGF-β3, as indicated by increase in chondrogenic gene expression (Agg and COL2). The differentiation state of ADSCS may directly affect paracrine factors secretion by ADSCS, which in turn influence the interaction between ADSCS and CHONS.

The dependence of cell proliferation and matrix synthesis on cell ratios in mixed cell culture along with the relatively weak response in conditioned-medium and bi-layered co-culture strongly suggests that local concentration and distribution of paracrine factors play a crucial role in mediating cell-cell crosstalk and the subsequent neo-tissue formation. In native tissue, the extracellular matrix (ECM) mediates soluble signaling through the storage, binding, presentation, and presentation of soluble growth factors. Growth factors secreted by cells may diffuse through the tissue, be internalized by neighboring cells, get physically entrapped within the matrix, or bind to specific ECM proteins. Growth factors that are known to mediate in chondrocyte metabolism such as FGFs, IGFs, and TGF-β's have been shown to bind to the ECM (van der Kraan et al. (2002) Osteoarthritis Cartilage 10:631-637; Taipale and Keski-Oja (1997) FASEB J. 11:51-59). Binding these cell-secreted growth factors to the ECM modulates the dynamics of autocrine and paracrine signaling, creating high local concentrations and limiting the diffusion of these factors within the ECM. Similarly, in the 3D hydrogel culture in this study, interactions of the paracrine factors with the hydrogel matrix as well as the newly synthesized cartilage ECM may result in localization of these factors in the hydrogel construct, limiting the amount of soluble factors that diffused into the media. This explains the differential results observed in mixed vs. bi-layer or conditioned medium culture. Likewise, the relatively weak response observed in our conditioned medium culture as compared to transwell co-culture reported in other studies may be due to the fact that a large portion of the paracrine signals were retained in the cell-gel construct as opposed to diffusing out into the media (Aung et al. (2011) Arthritis Rheum. 63:148-158).

Overall, this study clearly demonstrated that ADSCS catalyzed cell proliferation and cartilage formation by neonatal CHONS in a dose-dependent manner. At 21 days, mixed cell culture with as low as 25% ADSCS resulted in GAG and collagen content that were higher than those in CHON alone group. Although we only examined cartilage formation for up to 21 days, our immunostaining results indicated that neo-cartilage formation increased over the course of culture, and that the mixed co-culture groups with a high ratio of ADSC seem to catch up with the groups containing low ratio of ADSCS. Therefore, it is likely that at a later time point, neo-cartilage formation in the 10C:90A group would surpass the CHON alone as well as other mixed co-culture groups with lower ADSC ratios. It is expected that neo-cartilage deposited by the cells would completely replace the 3D hydrogel over a longer period of culture time, leading to the formation of a heterogeneous and mechanically functional neo-cartilage tissue.

The concept of utilizing stem cells to catalyze tissue formation by primary cells for tissue regeneration is not limited to application in cartilage but can be applied to other tissue types as well. By using different 3D biomimetic hydrogel culture models, we demonstrated the importance of intercellular distance and cell distribution in mediating the interactions of the two cell types, showing that the extent of cell proliferation and cartilage matrix production and organization were tightly regulated by these two variables. Our findings provide new insight into the design of 3D culture systems to probe cell-cell interactions, highlighting the advantages of using a 3D bio-mimetic hydrogel to examine cell-cell interactions in a physiologically relevant manner. In addition, our results also emphasized the relevance of manipulating cell-cell interactions in tissue engineering applications, highlighting the possibility of co-delivering small amount of neonatal chondrocytes from an autologous source with ADSCS to catalyze cartilage formation as a novel strategy to enhanced cartilage tissue repair and regeneration.

Materials and Methods

Cell Isolation and Culture

Chondrocytes: Hyaline articular cartilage was dissected from the femoropatellar groove of two stifle joints from a three-day old calf (Research 87, Marlborough, Mass.). The cartilage was sliced into thin pieces and digested in 1 mg/mL collagenase type II and type IV in high glucose DMEM supplemented with 100 U/mL penicillin and 0.1 mg/mL streptomycin for 24 hours at 37° C. The cell suspension was filtered through a 70 μm nylon mesh, washed in DPBS and centrifuged at 460 g for 15 minutes for three times, and counted with a hemocytometer. The bovine articular chondrocytes (bACs) were then suspended in freezing media (DMEM supplemented with 10% dimethyl sulfoxide (DMSO) and 50% fetal bovine serum (FBS), frozen at 1° C./minute, and stored in liquid nitrogen.

Adipose-derived stem cells: Adult human adipose-derived stem cells (ADSCS) were isolated from excised human adipose tissue with informed consent as previously described (Zuk et al. (2001) Tissue Eng. 7:211-228; herein incorporated by reference). ADSCS were expanded for 4 passages in high glucose DMEM supplemented with 5 ng/mL basic fibroblast growth factor (bFGF), 100 U/mL penicillin, and 0.1 mg/mL streptomycin.

3D Hydrogel Co-Culture

On the day of cell encapsulation, bACs were thawed, recounted and used without further expansion. Cells were suspended at 15×10⁶ cells/mL in a hydrogel solution consisted of 7% weight/volume (w/v) poly(ethylene glycol diacrylate) (PEGDA, MW=5000 g/mole), 3% w/v chondroitin sulfate-methacrylate (CS-MA), and 0.05% w/v photo-initiator (Irgacure D 2959; Ciba Specialty Chemicals) in DPBS. Cell-hydrogel suspension was pipetted into cylindrical gel mold with 75 μl volume and exposure to UV light (365 nm wavelength) at 3 MW/m² for 5 minutes to induce gelation. To create bi-layered hydrogel, cell-hydrogel suspension (37.5 μl each) of one cell type was deposited into the cylindrical gel mold and photo-crosslinked before deposition of the next cell-hydrogel layer. To prevent direct cell-cell contact, the two cell-hydrogel layers were separated by an acellular hydrogen layer (10 μl). The UV exposure times for the three sequential layers were 3, 2, and 5 minutes.

Co-Culture Models

To examine the effects of different local paracrine signals on cell fate, bACs and hADSCS were co-cultured in three different co-culture models: (1) Mixed co-culture in a single-layered hydrogel in 4 mixing ratios of bACs and hADSCS (75C:25A, 50C:50A, 25C:75A, 10C:90A); bACs alone and hADSCS alone were included as controls; (2) bi-layered co-culture with equal number of bACs and hADSCS confined to its own layer (bi-bAC and bi-hADSC); and (3) each of the cell types encapsulated in 3D hydrogels alone with supplementation of conditioned medium from the other cell type encapsulated in 3D (CM-bAC and CM-hADSC). The conditioned medium was collected every two days, filtered through a 0.2 μm mesh, and diluted with an equal volume of fresh chondrogenic medium. All samples were cultured in chondrogenic medium (high-glucose DMEM containing 100 nM dexamethasone, 50 μg/ml ascorbate-2-phosphate, 40 μg/ml proline, 100 μg/ml sodium pyruvate, 100 U/mL penicillin, 0.1 mg/mL streptomycin, and ITS Premix (5 μg/ml insulin, 5 μg/ml transferrin, 5 ng/ml selenious acid, BD Biosciences)) supplemented with 10 ng/ml TGF-β3 for 3 weeks.

Gene Expression Analysis

Total RNA was extracted from cell-hydrogel constructs (n=3) using TRIzol and the RNeasy mini kit (Qiagen). One mg of RNA from each sample was reversed transcribed into cDNA using the Superscript First-Strand Synthesis System (Invitrogen). Real-time polymerase chain reaction (PCR) was performed on an Applied Biosystems 7900 Real-Time PCR system using SYBR green master mix (Applied Biosystems) with the primers listed in Table 1. Human- and bovine-specific primers were used to quantify gene expression of chondrogenic markers including Type II collagen (COL2) and aggrecan (Agg) as well as fibroblastic marker type I collagen (COL1) using ΔΔCt method. Gene expression levels were normalized internally to GAPDH. Relative fold changes represent changes in gene expressions compared with bACs alone group (for bovine-specific gene expressions) and hADSCS alone group (for human-specific gene expressions) at day 1.

TABLE 1
List of species-specific primers used for real-time polymerase
chain reaction.

| Gene | | | |
|---|---|---|---|
| Name | Species | Primer Sequence | GenBank No. |
| GAPDH | Human | F: 5′ CGCTCTCTGCTCCTCCTGTT 3′ | NM_002046.3 |
| | | (SEQ ID NO: 1) | |
| | | R: 5′ CCATGGTGTCTGAGCGATGT 3′ | |
| | | (SEQ ID NO: 2) | |
| | Bovine | F: 5′ AGATGGTGAAGGTCGGAGTG | NM_001034034.1 |
| | | (SEQ ID NO: 3) | |
| | | R: 5′ GATCTCGCTCCTGGAAGATG | |
| | | (SEQ ID NO: 4) | |
| Aggrecan | Human | F: 5′ TGAGGAGGGCTGGAACAAGTACC 3′ | NM_001135.3 |
| (Agg) | | (SEQ ID NO: 5) | |
| | | R: 5′ GGAGGTGGTAATTGCAGGGAACA 3′ | |
| | | (SEQ ID NO: 6) | |
| | Bovine | F: 5′ CACCACAGCAGGTGAACTAGA 3′ | NM_173981.2 |
| | | (SEQ ID NO: 7) | |
| | | R: 5′ GCTTGCTCCTCCACTAATGTC 3′ | |
| | | (SEQ ID NO: 8) | |
| COL2A1 | Human | F: 5′ TCACGTACACTGCCCTGAAG 3′ | NM_001844.4 |
| (COL2) | | (SEQ ID NO: 9) | |
| | | R: 5′ TTGCAACGGATTGTGTTGTT 3′ | |
| | | (SEQ ID NO: 10) | |
| | Bovine | F: 5′ GTGGGGCAAGACTATGATCG 3′ | NM_001113224.1 |
| | | (SEQ ID NO: 11) | |
| | | R: 5′ TGCAATGGATTGTGTTGGTT 3′ | |
| | | (SEQ ID NO: 12) | |
| COL1A2 | Human | F: 5′ AGGGCAACAGCAGGTTCACTTACA 3′ | NM_000089.3 |
| (COL1) | | (SEQ ID NO: 13) | |
| | | R: 5′ AGCGGGGGAAGGAGTTAATGAAAC 3′ | |
| | | (SEQ ID NO: 14) | |
| | Bovine | F: 5′ ACATTGGCCCAGTCTGTTTC 3′ | NM_174520.2 |
| | | (SEQ ID NO: 15) | |
| | | R: 5′ GGGAGGGGGAGTGAATTAAA 3′ | |
| | | (SEQ ID NO: 16) | |

Biochemical Analysis

Cell-hydrogel constructs (n=4) were weighed wet, lyophilized, weighed dry, and digested in papainase solution (Worthington) at 60° C. for 16 hours. DNA content was measured using the PicoGreen assay (Invitrogen, Molecular Probes) using Lambda phage DNA as standard. Glycosaminoglycan (GAG) content was quantified using the 1,9-dimethylmethylene blue (DMMB) dye-binding assay with shark chondroitin sulfate as a standard. Total collagen content was determined using acid hydrolysis followed by reaction with p-dimethylaminobenzaldehyde and chloramines. T. Collagen content was estimated by assuming a 1:7.46 hydroxyproline:collagen mass ratio. The interaction index, which is the measured matrix content (DNA, sGAG, or collagen) in the mixed co-culture group normalized by the expected matrix content based on the measured matrix content per in the bAC and hADSC alone groups, was calculated. An interaction index of greater than 1 indicates that the resulting matrix content is higher than expected, while an interactions index of lower than one indicates that the resulting matrix content is lower than expected. An interaction index of one indicates that the resulting matrix content was the same as expected.

Histological Analysis

Cell-hydrogel constructs (n=2) were fixed in 4% paraformaldehyde overnight and stored in 70% ethanol at 4° C. until processed. Constructs were then embedded in paraffin and processed using standard histological procedures. For immunostaining, enzymatic antigen retrieval was performed by incubation in 0.1% Trypsin at 37° C. for 15 minutes. Sections were then blocked with blocking buffer consisting of 2% goat serum, 3% BSA and 0.1% Triton X-100 in 1×PBS, followed by incubation in rabbit polyclonal antibody to collagen type I or II (Abcam) overnight at 4° C. and secondary antibody (Alexa Fluor 488 goat anti-rabbit, Invitrogen) incubation for an hour at room temperature. Nuclei were counterstained with DAPI mounting medium (Vectashield) and images were taken with a Zeiss fluorescence microscope. Sections without primary antibody incubation served as negative controls. A custom image processing program was written in MATLAB (The MathWorks) to quantify the number and size of the cartilage nodules.

Cell Tracking Using Membrane Labeling

To track cell distribution within the hydrogel constructs over time, hADSCS were labeled with red fluorescent dye (PKH26, Sigma) prior to encapsulation at a concentration of 4 μM for 4 minutes following manufacturer's protocol. Labeled hADSCS were co-cultured with bACs in the mixed co-culture hydrogel model at different cell ratios for 21 days (n=3). Samples were fixed in 4% paraformaldehyde overnight, submerged in 30% sucrose solution for 24 hours, embedded in Tissue-Tek (Sakura Finetek), and frozen in liquid nitrogen. Cryosections (12 μm) were washed in DPBS and collagen II and cell nuclei were stained using the immunostaining procedures described above.

Mechanical Testing

Unconfined compression tests were conducted using an Instron 5944 materials testing system (Instron Corporation, Norwood, Mass.) fitted with a 10 N load cell (Interface Inc., Scottsdale, Az). Cell-hydrogel constructs were tested on day 1 and day 21 of culture (n=4). During testing, cell-hydrogel constructs were submerged in a PBS bath at room temperature. Constructs were compressed at a rate of 1% strain/second to a maximum strain of 15%. Stress versus strain curves were created and curve fit using a third order polynomial equation. The compressive tangent modulus was determined from the curve fit equation at strain values of 15%.

Statistical Analysis

GraphPad Prism (Graphpad Software, San Diego) was used to perform statistical analysis. One- or two-way analysis of variance and pairwise comparisons with Tukey's post-hoc test were used to determined statistical significance (p<0.05). Data was represented as mean±standard deviation of at least three biological replicates.

### Example 2

### Harnessing the Synergy Between Neonatal Chondrocytes and Adipose-Derived Stem Cells for Cartilage Regeneration In Vivo

Introduction

Although using a mixture of ADSCs and NChons for cartilage repair would ameliorate issues related to donor scarcity of NChons, the minimal ratio of NChons and the microenvironmental cues needed for robust cartilage repair remains unknown. Furthermore, the in-vivo efficacy of mixed cell populations for cartilage repair has yet to be demonstrated.

To validate the potential of transplanting a mixed population of ADSCs and NChons for cartilage repair, we sought to determine the minimal ratio of NChons to ADSCs required to induce robust neocartilage formation both in vitro and in vivo. Both chondrocytes and ADSCs have been shown to be sensitive to changes in O₂ tension, which can influence cell proliferation, phenotype, and ECM deposition (Coyle et al. (2009) J Orthop Res 27(6):793-799; Murphy et al. (2004) J Cell Physiol 199(3):451-459; Wang et al. (2005) J Cell Physiol 204(1):184-191; and Xu et al. (2007) Tissue Eng 13(12):2981-2993). However, how hypoxia and soluble factor environment influence the cell interactions between NChons and ADSCs remains unknown. Given the hypoxic microenvironment in cartilage tissue, we further examined the effects of O₂ concentration and soluble factors on the synergy between these two cell types. To evaluate in vivo efficacy of cartilage regeneration, we transplanted 3D biomimetic hydrogels containing a mixed population of ADSCs and NChons in vivo using a subcutaneous mouse model and evaluated the resulting cartilage tissue formation for up to 12 weeks.

Results

As Few as 2% NChons are Sufficient in Co-Culture to Catalyze Cartilage Formation.

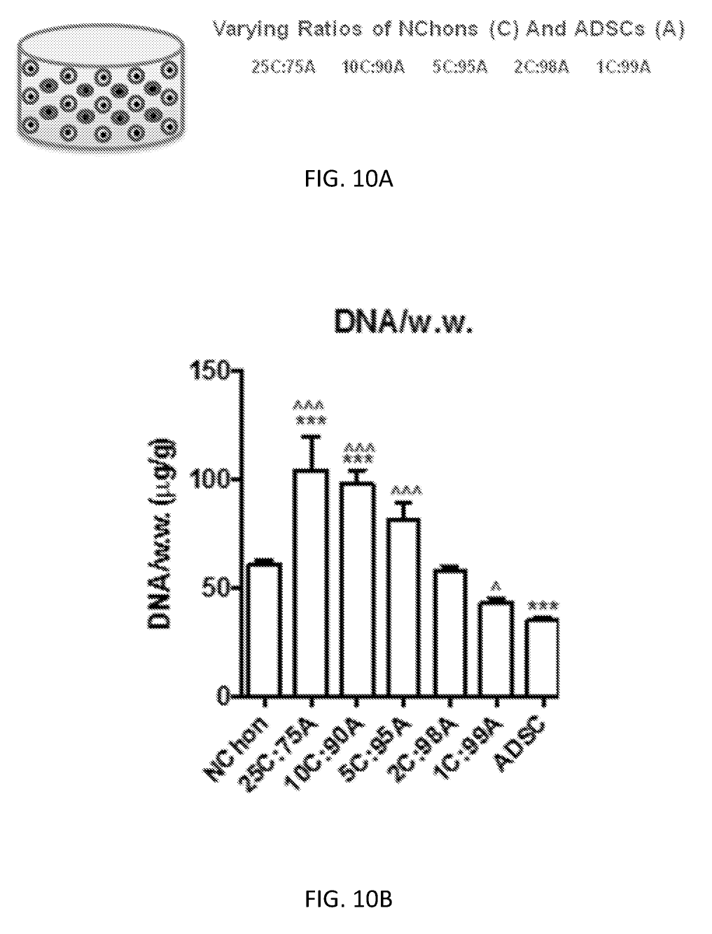

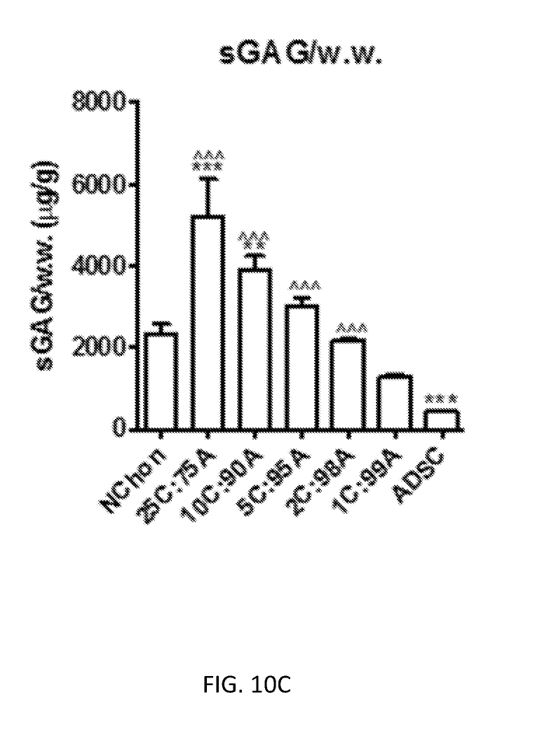

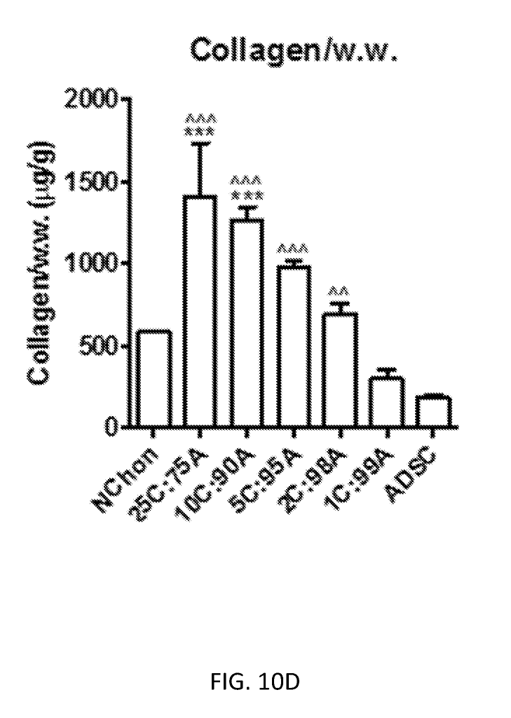

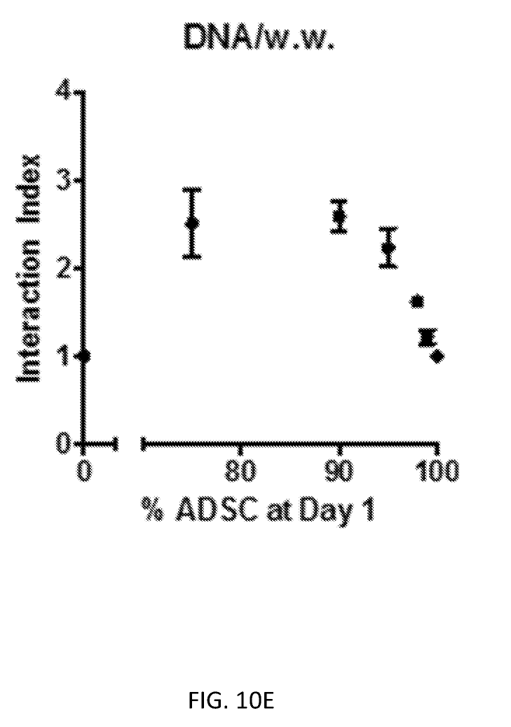

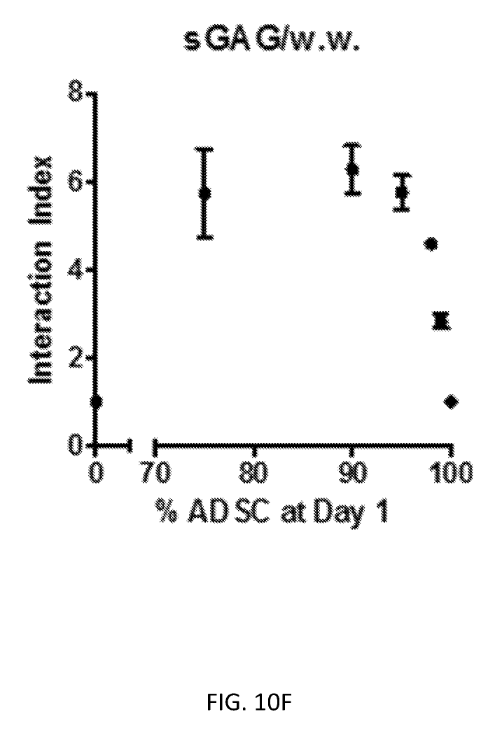

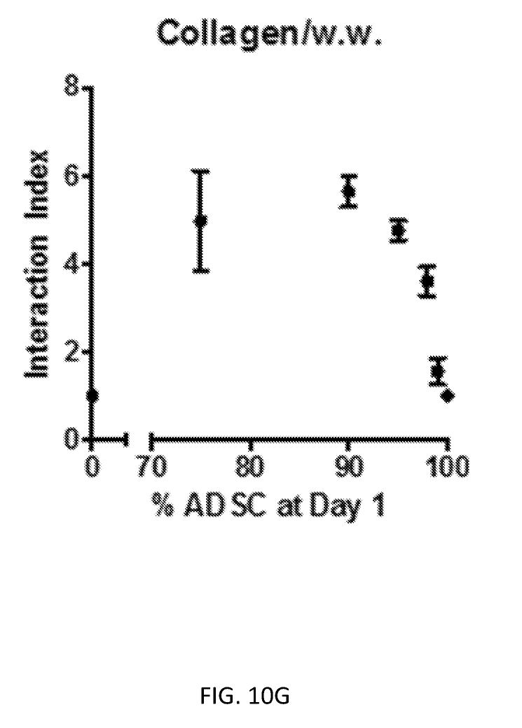

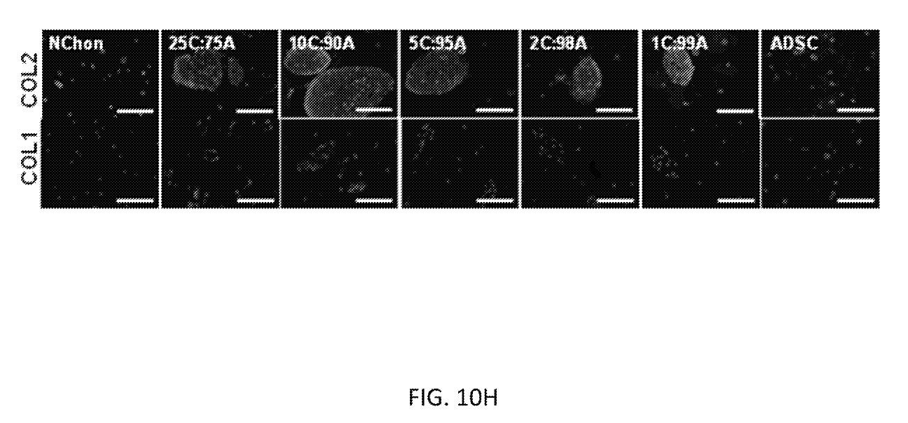

To determine the minimal ratio of NChons to ADSCs needed for effective cartilage formation, the two cell types were co-cultured in 3D biomimetic hydrogels with various percentages of NChons (25%, 10%, 5%, 2%, and 1%; FIG. 10A). The hydrogel consisted of poly(ethylene) glycol diacrylate (PEGDA) and chondroitin sulfate methacrylate (CSMA), which crosslinked via UV light exposure in the presence of a photoinitiator. We incorporated chondroitin sulfate into our hydrogels to mimic cartilage ECM and to facilitate cell-mediated degradation and promote tissue growth (Varghese et al. (2008) Matrix Biol 27(1):12-21; Hwang N S, et al. (2007) Febs Letters 581:4172-4178). Total cell seeding density was maintained constant at 15 million/mL, and all cell-hydrogel constructs were cultured in chondrogenic medium containing TGF-β3 (Materials and Methods).

Remarkably, mixed co-culture with as few as 2% NChons resulted in neocartilage formation that was equivalent to that generated by control populations of 100% NChons in terms of cell number (DNA; FIG. 10B), sulfated glycosaminoglycan (sGAG; FIG. 10C), and collagen (FIG. 10D) content per wet weight. Furthermore, co-culture with 10% or 25% NChons yielded significantly higher amounts of cartilage ECM (sGAG and collagen) than did the 100% NChon control (up to 2.4-fold; FIGS. 10B-10D). To further quantify the effects of varying cell ratio on the synergy between NChons and ADSCs, we calculated the interaction index, a normalized comparison of the measured over the expected cartilage ECM content (Acharya et al. (2012) J Cell Physiol 227(1):88-97); interaction indices >1 reflect synergy (Methods). We found that the interaction index peaked using 5-25% NChons, up to ˜2.5 for DNA and ˜6 for sGAG and collagen content (FIGS. 10E-10G). Lowering the NChon percentage below 5% led to a drop in interaction synergy (FIGS. 10E-10G).

Consistent with our biochemical results, immunostaining of type II collagen, a major ECM component found in articular cartilage, revealed the formation of cartilage nodules in hydrogels containing 1-25% NChons in mixed culture (FIG. 10H). At day 21, hydrogels co-cultured with 10% NChons displayed the largest cartilage nodules (˜200 μm); even cultures containing 1-2% NChons produced large cartilage nodules (˜100 μm; FIG. 10H). Cell tracking revealed that ADSCs (red) resided outside the cartilage nodules in all hydrogels cultured with mixed cell populations (FIG. 10H), suggesting that neocartilage was contributed by NChons and that ADSCs stimulated NChons to proliferate and produce cartilage ECM. The intense type II collagen staining together with minimal type I collagen staining (FIG. 10H) indicated that the cartilage generated through mixed co-culture possesses the therapeutically desirable articular cartilage phenotype instead of the fibrocartilage phenotype.

TGF-β3 is Required for Catalyzed Cartilage Formation in Mixed Populations of ADSCs and NChons.

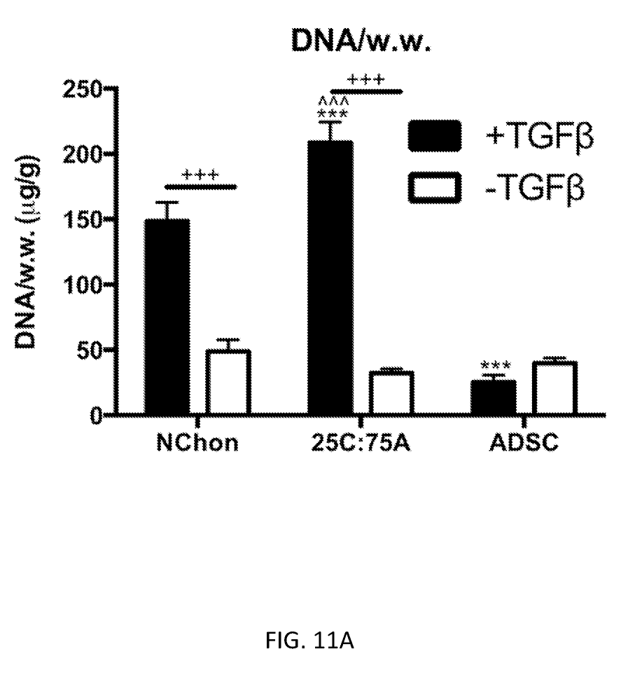

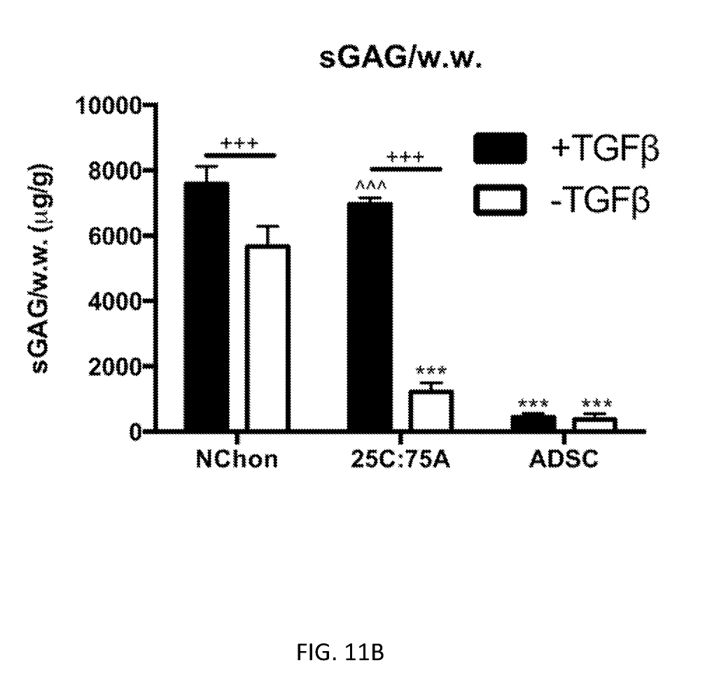

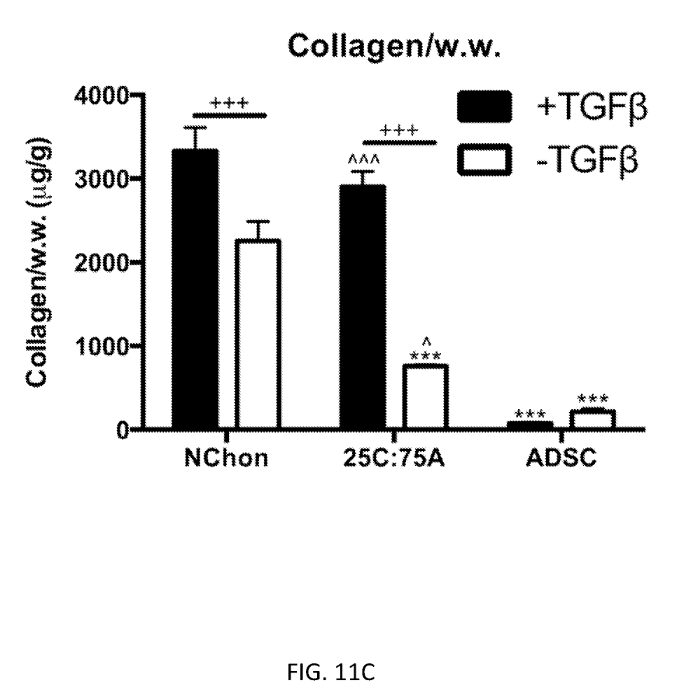

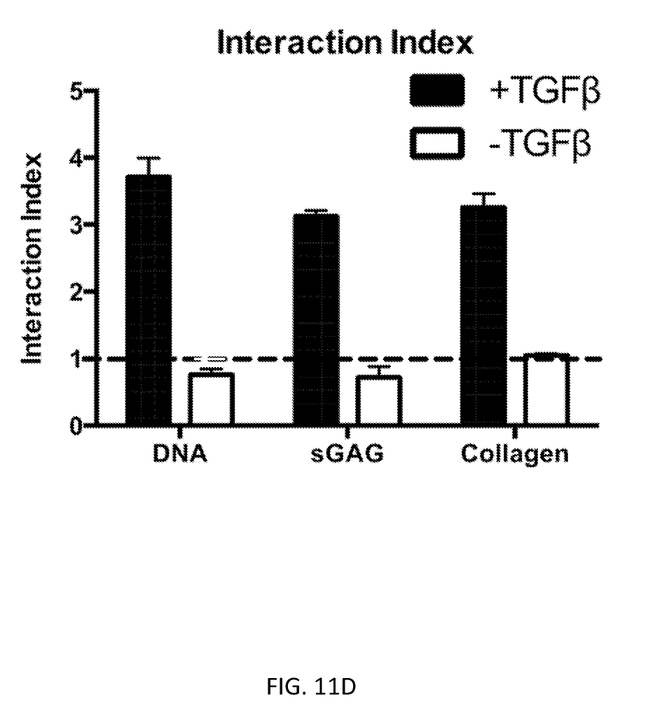

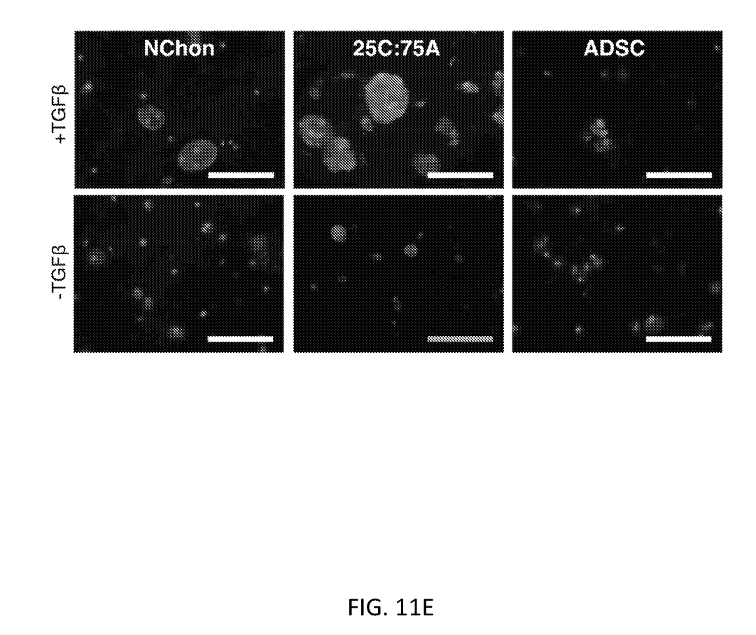

Interaction synergy in mixed co-culture did not persist without TGF-β3, as indicated by significantly lower DNA (FIG. 11A), sGAG (FIG. 11B), and collagen content (FIG. 11C) compared to those produced by mixed co-culture under TGF-β3 induction. Interaction index for DNA, sGAG, and collagen also indicated that synergy only occurred in mixed co-culture with TGF-β3, with interaction indices greater than 1 (FIG. 11D). Consistent with biochemical analysis, immunostaining for collagen type II revealed enhanced cartilage nodule formation in mixed co-culture with TGF-β3 only (FIG. 11E).

Synergy Between ADSCs and NChons Persists Under Hypoxia.

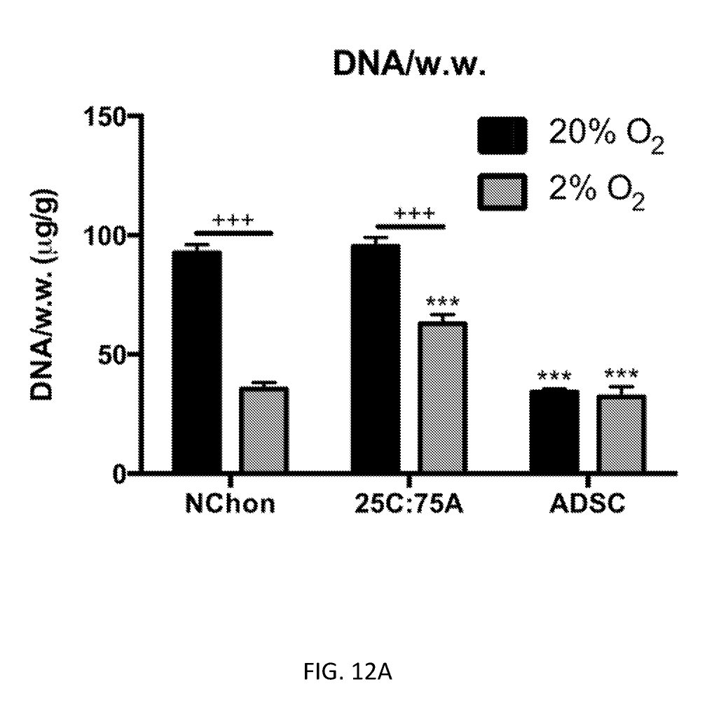

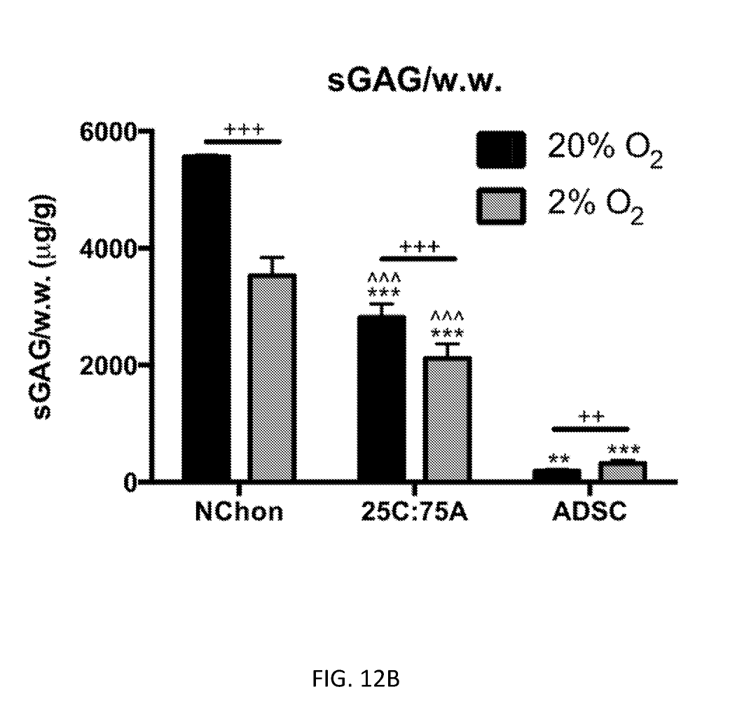

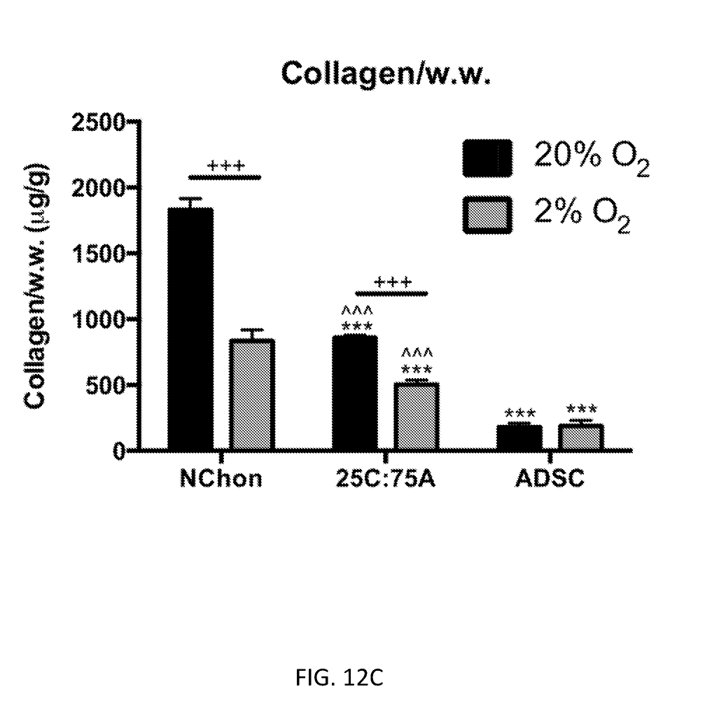

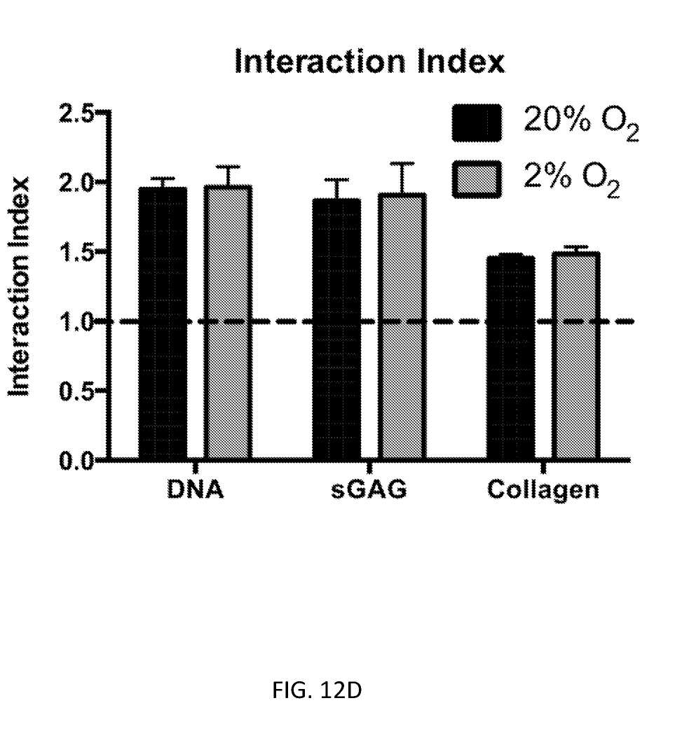

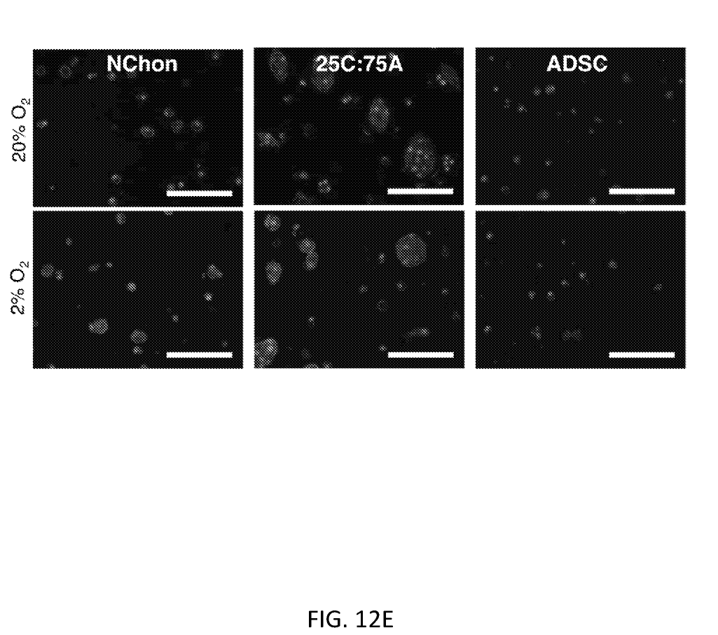

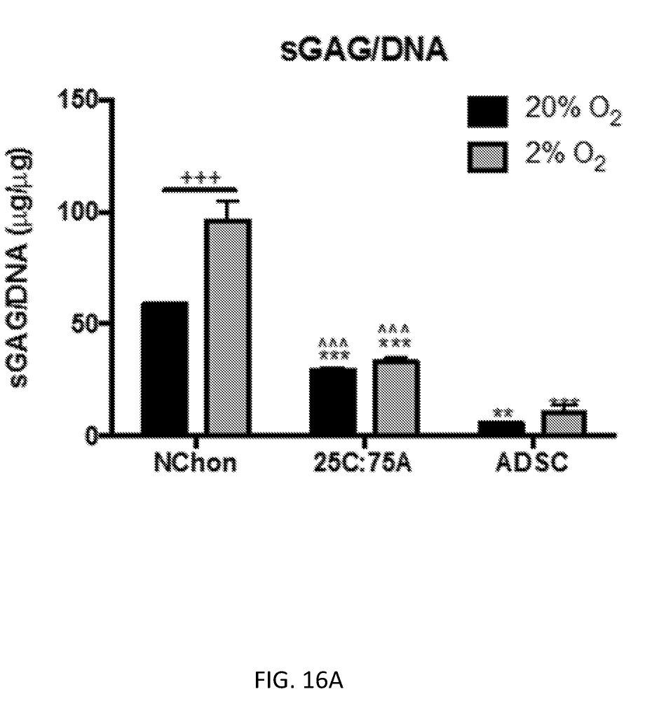

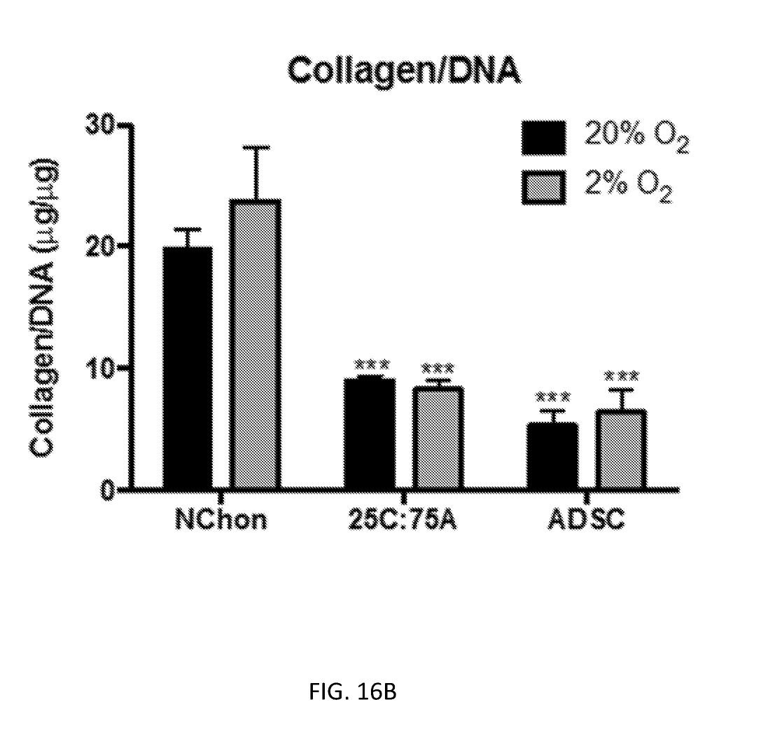

Cartilage is a hypoxic environment in which local O₂ tension ranges from 1% to 7% (Silver (1975) Philos Trans R Soc Lond B Biol Sci 271(912):261-272), levels that are much lower than those employed in standard culture conditions (20%). To better predict the efficacy of mixed cell culture for cartilage repair in vivo, we considered the effects of low O₂ tension on cell fate and ECM production. Thus, NChons and ADSCs were co-cultured in 3D biomimetic hydrogels exposed to 2% or 20% O₂ in the presence of TGF-β3 for 14 days in vitro. While changes in the soluble-factor microenvironment substantially affected the interaction between ADSCs and NChons (FIG. 11), synergy between ADSCs and NChons persisted under hypoxia in the presence of TGF-β. Although total proliferation and cartilage ECM production decreased relative to 20% O₂ (FIGS. 12A-12C), interaction synergy in mixed co-culture was retained at 2% O₂, as indicated by interaction indices for DNA, sGAG, and collagen content (FIG. 12D). Notably, on a per-cell basis, sGAG and collagen per DNA were comparable in mixed co-culture at 2 and 20% O₂, indicating that cartilage ECM production per cell was not affected at hypoxia (FIG. 16). Cartilage nodules were observed at both O₂ concentrations (FIG. 11E). Taken together, these results supported the potential efficacy of mixed co-culture for tissue regeneration in a hypoxic cartilage environment.

Catalyzed Cartilage Formation Via NChons and ADSCs is Sustained In Vivo.

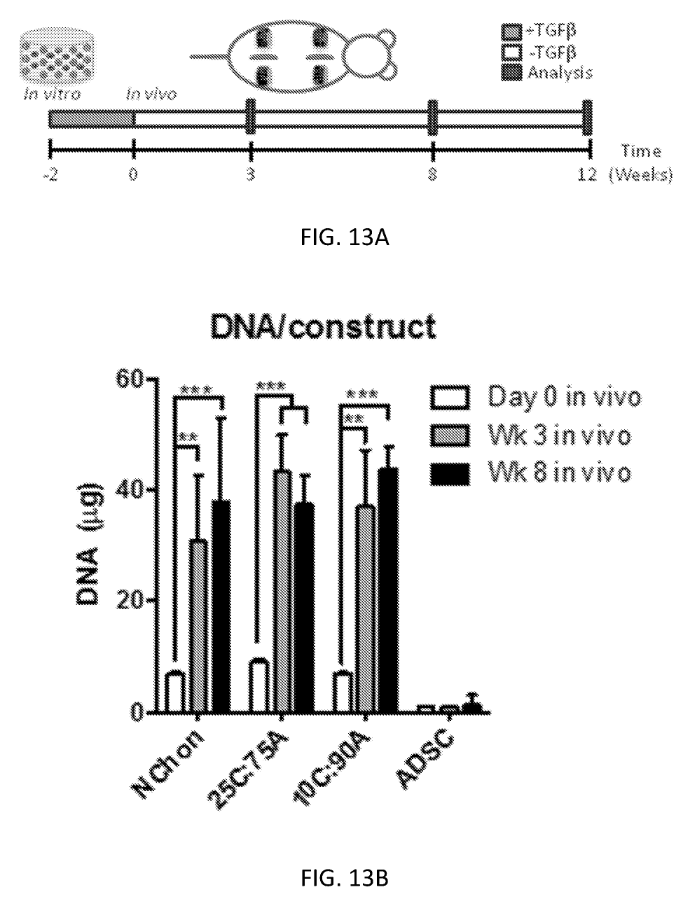

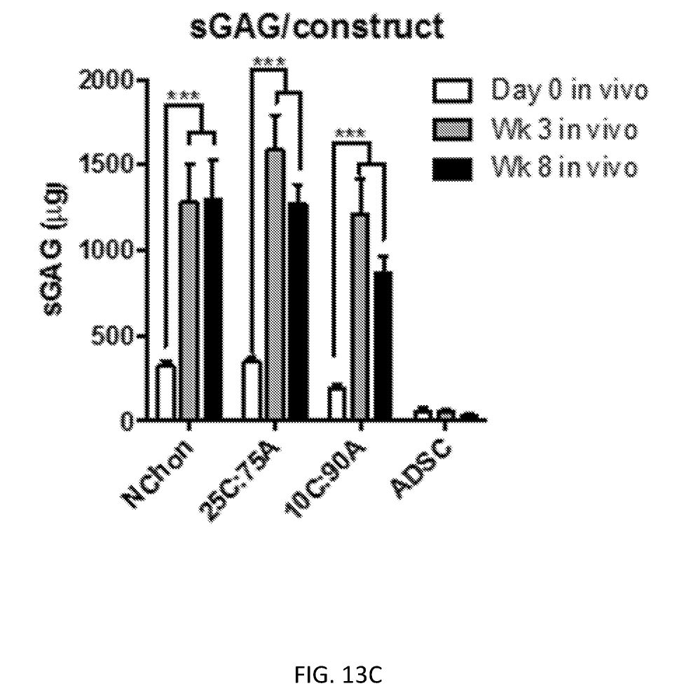

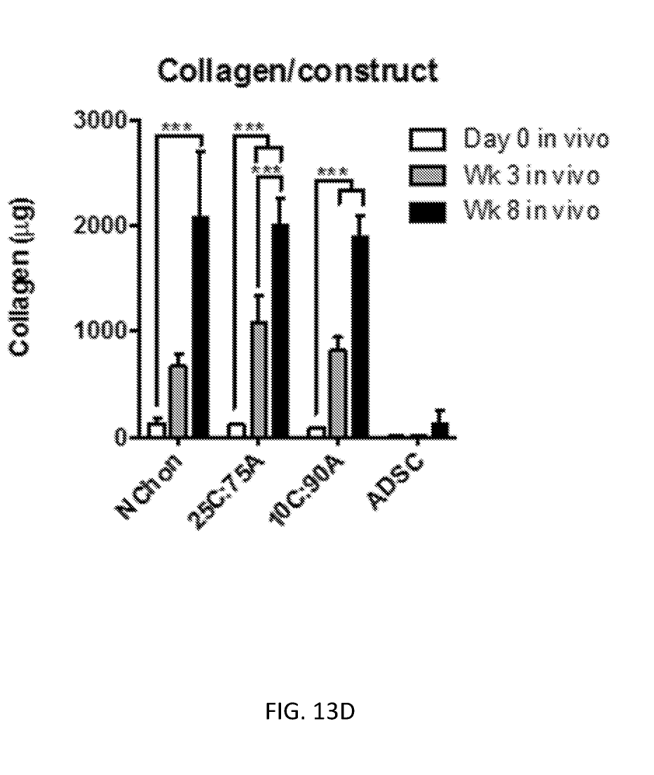

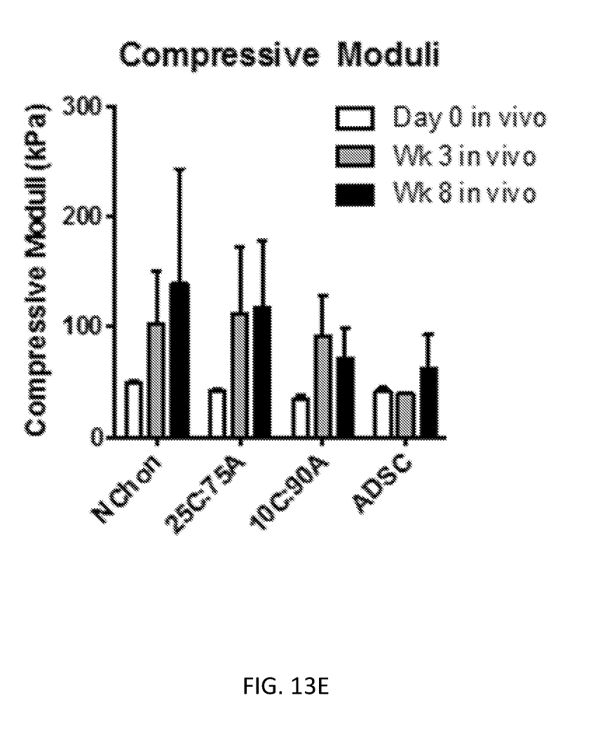

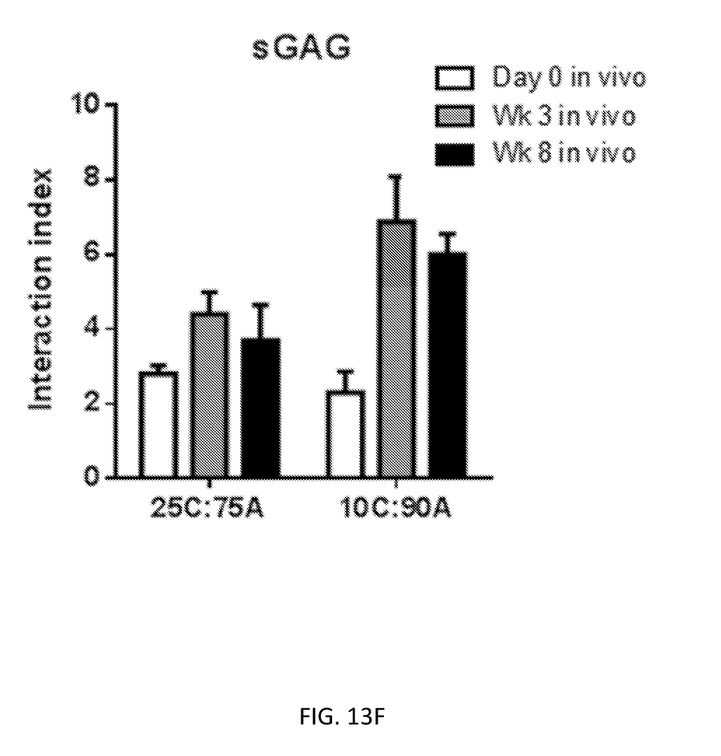

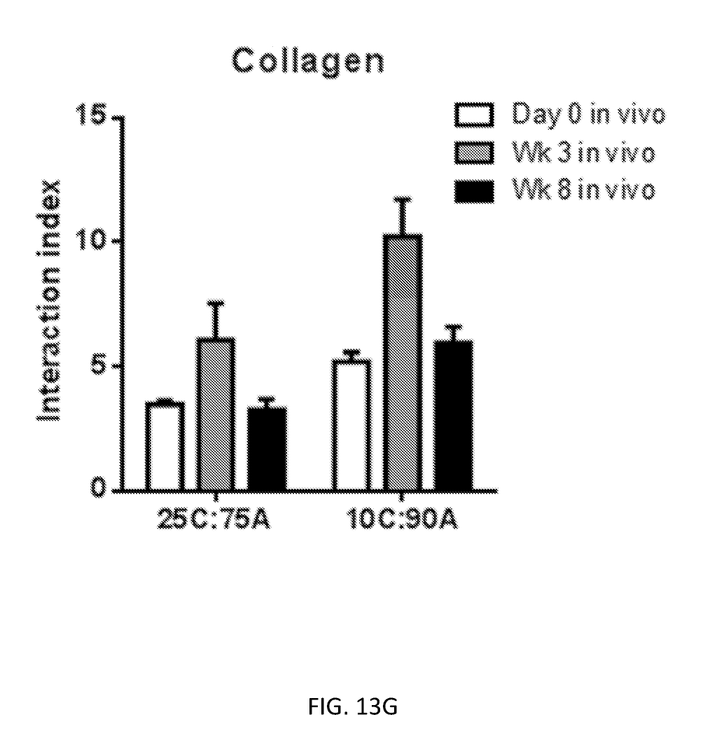

We extended our in-vitro investigations to further assess the ability of ADSCs to catalyze cartilage tissue formation by NChons in vivo. Specifically, we transplanted 3D hydrogels containing mixed co-cultures of 25% or 10% NChons (25C:75A, 10C:90A) into an athymic mouse subcutaneous model using female nude mice (9 weeks old, see Materials and Methods). We selected the 25% and 10% NChon co-cultures based on our observations that these cell ratios yielded highest synergy and maximal cartilage ECM production in vitro (FIG. 10). Pure cultures of ADSCs or NChons were included as controls. Since synergy was observed only with TGF-β in vitro (FIG. 11), cell-hydrogel constructs were pre-cultured in vitro for 2 weeks in chondrogenic medium supplemented with 10 ng/mL TGF-β3 prior to implantation and the resulting cartilage tissue formation was evaluated after up to 12 weeks in vivo (FIG. 13A).

In the mixed co-cultures, most cell proliferation (FIG. 13B) and sGAG deposition (FIG. 13C) occurred within the first 3 weeks, while total collagen production continued to increase up to 8 weeks (up to 21-fold increase compared to day 0 in vivo, FIG. 13D). Total DNA, sGAG, and collagen content were comparable in the mixed co-cultures and the NChon control, but were significantly lower in the ADSC control (FIGS. 13B-13D). The compressive moduli of cell-hydrogel constructs increased after in-vivo implantation in all groups (FIG. 13E). Further, interaction indices demonstrated that NChon-ADSC synergy continued to increase after in-vivo transplantation (FIGS. 13F, 13G). Interaction indices were higher for 10C:90A group than 25C:75A group, and peaked after 3 weeks in vivo, with maximal indices of 6.8 and 10.2 for sGAG (FIG. 13F) and collagen (FIG. 13G), respectively.

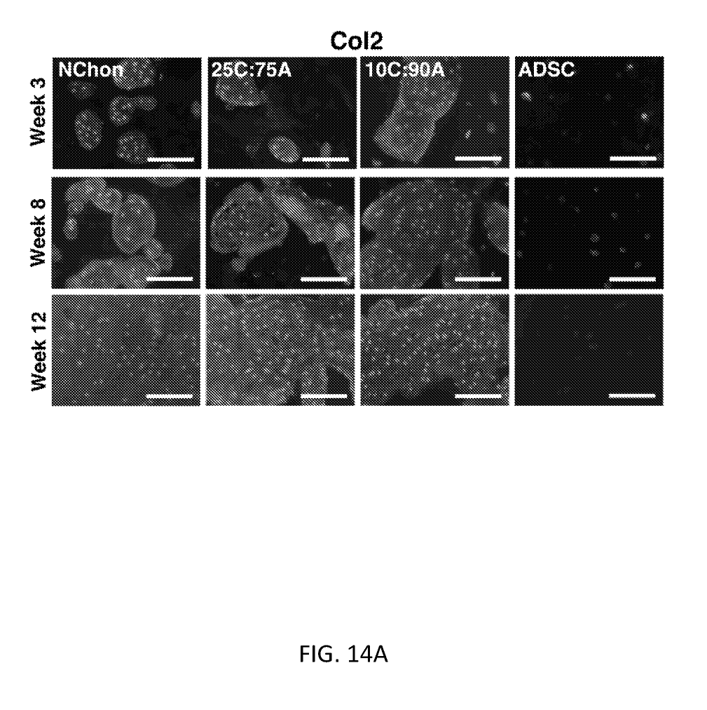

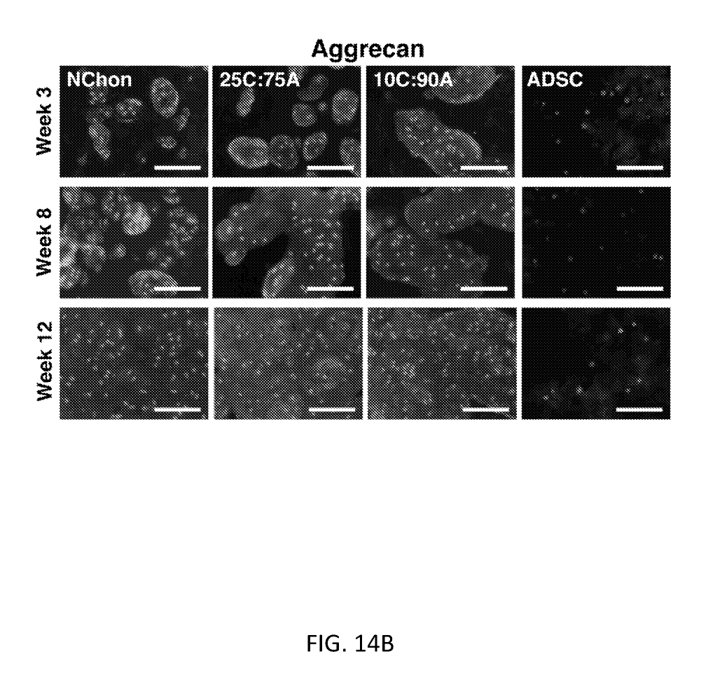

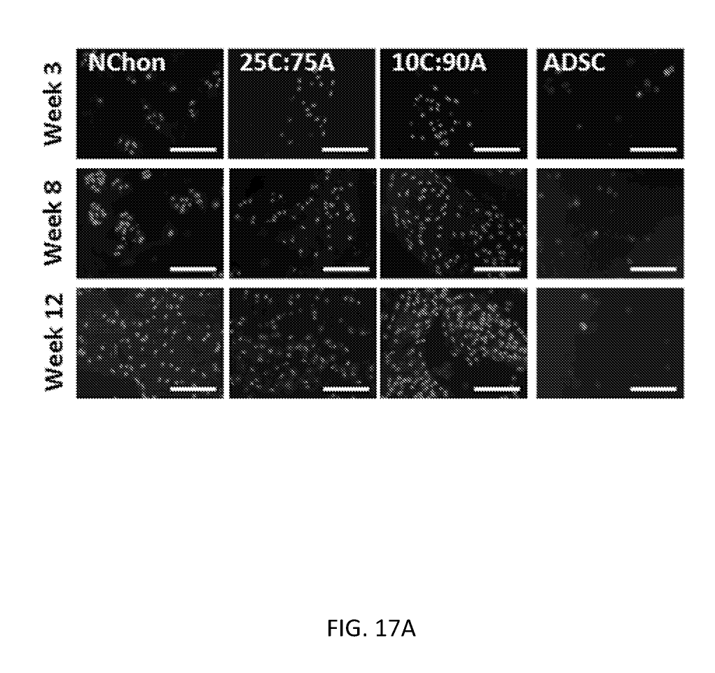

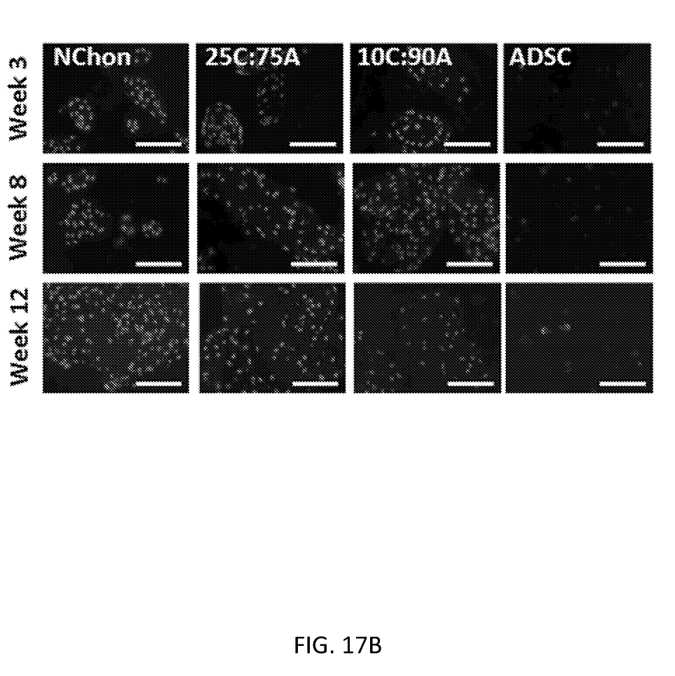

Articular cartilage matrix is characterized by abundant type II collagen and aggrecan. Immunostaining of newly deposited matrix against type II collagen (FIG. 14A) and aggrecan (FIG. 14B) revealed that hydrogels containing mixed cell populations or NChon alone were gradually replaced with neocartilage rich in type II collagen and aggrecan. Little type I or type X collagen was detected (FIG. 17), indicating that the resulting cartilage was not fibroblastic or hypertrophic cartilage phenotype. Taken together, our observations demonstrate that the robust synergistic interactions between NChons and ADSCs in mixed co-cultures persist in vivo in the absence of additional TGF-β3 supplementation beyond the 2-week in-vitro culture. Moreover, using as few as 10% NChons in the mixed co-culture allows almost complete matrix remodeling by the newly formed articular cartilage by 12 weeks post-implantation.

Discussion

Cell-based therapy offers a promising solution for cartilage repair, but its clinical application remains limited due to the lack of abundant cell sources that yield the articular/hyaline cartilage phenotype. Here, we revisited the concept of optimal cell-source selection for cartilage repair by seeking to engineer interactions between NChons and ADSCs in 3D biomimetic hydrogels to maximize cartilage regeneration while minimizing the number of NChons required. We chose ADSCs because they are abundantly available and can undergo chondrogenesis to produce cartilage tissue (Zuk et al. (2001) Tissue Eng 7(2):211-228; Guilak (2004) Biorheology 41(3-4):389-399). In addition, ADSCs may be harvested in a one-step procedure in the operating room with minimal ex-vivo manipulation, making them an attractive cell source for cell-based therapy (Jurgens et al. (2008) Cell Tissue Res 332(3):415-426; Jurgens et al. (2013) Biores Open Access 2(4):315-325). The objectives in the current investigation were to optimize the synergy between NChons and ADSCs while minimizing the number of NChons in mixed co-culture, to characterize the response of cell-hydrogel constructs to O₂ and soluble factors, and to assess the efficacy of mixed co-culture for cartilage tissue formation in vivo.

Our results showed that we could achieve robust articular cartilage formation using significantly reduced number of NChons by replacing majority of NChons with ADSCs. Interaction synergy in mixed co-cultures peaked with populations of 5-25% NChons, leading to total cartilage ECM production that surpassed that of the 100% NChon control (FIGS. 10B-10G). Remarkably, mixed co-cultures with as few as 2% NChons led to cartilage ECM production that was comparable to that of the 100% NChon control (FIGS. 10C, 10D). Increasing the percentage of ADSCs in mixed cell populations likely increased the concentration of paracrine factors secreted by the ADSCs into the 3D hydrogel, thus enhancing the catalysis of NChons by ADSCs. Our observation of an optimal range (5-25% NChons) reflects a trade-off between increasing paracrine effects and decreasing numbers of NChons within the 3D hydrogel matrix that are available to initiate the formation of cartilage nodules. However, we evaluated outcome after 3 weeks of in-vitro mixed co-culture, and it is likely that mixed cell populations containing 1% NChons or less may catch up during longer co-culture to yield cartilage ECM production that is comparable to that of the NChon control.

Microenvironmental parameters such as O₂ tension and soluble factors are also crucial modulators of cell behavior. Therefore, we sought to understand their effects on NChon-ADSC interactions to better predict the outcome of tissue regeneration. Cartilage is a hypoxic microenvironment, and hypoxia was previously shown to modulate MSC chondrogenesis, chondrocyte phenotype, and matrix-production (Coyle et al. (2009) J Orthop Res 27(6):793-799; Malladi et al. (2006) Am J Physiol Cell Physiol 290(4):C1139-1146). A recent study demonstrated that low O₂ tension (5%) reduced cell proliferation but not cartilage ECM production in co-cultured adult articular chondrocytes and bone marrow-derived MSCs in poly(ε-caprolactone) (PCL) scaffolds (Meretoja et al. (2013) Biomaterials 34(17):4266-4273). Here we observed that although hypoxia (2% O₂) reduced cell proliferation and total cartilage formation in co-cultured NChons and ADSCs, interaction synergy was retained, as revealed by comparable interaction indices for cell proliferation and cartilage ECM production (FIG. 12D). These results underscore the promise of utilizing mixed populations of NChons and ADSCs to repair articular cartilage. Importantly, we detected enhanced cell proliferation and cartilage ECM production only in mixed co-culture under TGF-β3 induction. Therefore, it is important to include TGF-β3 in ex-vivo co-cultures prior to implantation, or perhaps to deliver TGF-β3 in vivo along with the cells and hydrogel to ensure optimal outcomes.

Interaction synergy also persisted during long-term in-vivo implantation without further addition of TGF-β3. In mixed co-cultures containing 25% or 10% NChons, DNA and sGAG increased most rapidly during the first 3 weeks in vivo; collagen deposition continued to increase at 8 weeks (FIGS. 13B-13D). Interaction indices continued to increase and were substantially higher after in-vivo implantation, emphasizing the persistence of interaction synergy in vivo. By 12 weeks, hydrogel matrices containing mixed co-cultures or pure NChon populations were substantially remodeled and replaced by cell-secreted cartilage nodules rich in proteoglycan (aggrecan) and type II collagen, but without fibroblastic or hypertrophic phenotypes. In contrast, pure ADSC populations showed decreased cell number and deposited little cartilage ECM in vivo. The drastic differences in cell number and cartilage ECM phenotypes and deposition between pure ADSC populations and mixed co-cultures observed here indicate that mixed co-cultures may circumvent shortcomings related to the use of stem cells in cartilage repair, such as hypertrophy, while harnessing their trophic effects on NChons to maximize cartilage production.

We chose to deliver mixed cell populations in injectable and photopolymerizable hydrogels, which offer several advantages for repairing cartilage defects. First, cells can be readily distributed throughout the hydrogel in a homogeneous manner prior to crosslinking, and then exposed to light to induce gelation to fill cartilage defects of any shape and size; in contrast, pre-fabricated scaffolds (e.g. collagen sponges) are often associated with uneven cell distributions or require additional time (˜hours) and a perfusion device to ensure that cells penetrate the scaffold (Meretoja et al. (2013) Biomaterials 34(17):4266-4273). Controlling cell distributions in 3D facilitates better manipulation of the extent of interaction and therefore leads to better control over outcome, particularly since we previously reported that cell-cell interactions are extremely sensitive to proximity (Lai et al. (2013) Sci Rep 3:355321). In the present investigation, we specifically selected a hydrogel composed of one synthetic and one bioactive polymer for two reasons. First, the synthetic polymer component of our 3D biomimetic hydrogel, poly(ethylene) glycol diacrylate, provides some baseline mechanical support (an initial compressive modulus of ˜30 kPa) during the initial phase of implantation before the cells are able to produce cartilage ECM. Second, the addition of chondroitin sulfate to the hydrogel network promotes cell-mediated degradation and remodeling. Our immunostaining analyses revealed that the cell-hydrogel constructs went through a gradual remodeling process, during which the hydrogel matrix was replaced by neocartilage rich in articular cartilage markers including type II collagen and aggrecan (FIG. 14).

In terms of clinical translation, combining ADSCs with NChons has several advantages over current approaches. First, this technique relieves issues of NChon scarcity by substituting NChons with a more abundant cell source. Furthermore, the use of allogeneic NChons may reduce outcome variability due to patient age and other conditions, thereby relaxing demographic criteria for patient selection. In addition, mixed cell populations may simplify the treatment to a one-step procedure and minimize ex-vivo manipulations. Allogeneic chondrocytes from neonatal donors may be stored in a cell bank and mixed with autologous ADSCs that are freshly isolated from the patient in the operating room (Jurgens et al. (2008) Cell Tissue Res 332(3):415-426; Jurgens et al. (2013) Biores Open Access 2(4):315-325).

While we have demonstrated that synergy between NChons and ADSCs lead to robust neo-cartilage production, the underlying mechanism for cell-cell interaction still remains unknown. Cell labeling and immunostaining results indicate that ADSCs catalyze neocartilage formation by NChons without direct cell-cell contact (FIG. 10H), suggesting that a paracrine effect was responsible. Future study should identify the paracrine factors secreted by ADSCs that catalyze cartilage formation. Identification of these paracrine factors may allow in vitro treatment of NChons prior to delivery or direct delivery of these factors along with NChons to catalyze cartilage ECM production, potentially eliminating the need for ADSC transplantation.

Overall, here we have demonstrated the potential of harnessing synergistic interactions between ADSCs and NChons to achieve robust, catalyzed cartilage formation in vitro and in vivo. Using as few as 2% NChons, mixed co-cultures of ADSCs and NChons generated amounts of neocartilage that were comparable to those from pure populations of NChons. This robust synergy and cartilage formation was also observed at low O₂ concentrations, supporting the efficacy of this technique in the hypoxic environment of cartilage defects. Synergy was highly dependent on the soluble-factor microenvironment, and TFG-β3 was required for catalyzed cartilage formation. Cartilage continued to form in vivo after a brief 2-week in-vitro culture with TFG-β3. Mixed cell populations with 10% NChons led to the extensive formation of neocartilage with the therapeutically desirable articular phenotype, almost completely degraded the original hydrogel matrix, and yielded neocartilage 12 weeks after transplantation in vivo in a subcutaneous mouse model.

Given the short period of TFG-β3 exposure used here, it would be interesting to investigate the feasibility of completely removing in-vitro culture for direct transplantation of mixed cell populations with TFG-β3 for cartilage repair. Moreover, the mechanical and biochemical properties of the 3D hydrogel scaffold could be further optimized to enhance synergy while providing initial mechanical and chemical cues. We have chosen to employ an athymic mouse model in this investigation as it allows proof-of-principle screening studies with a larger sample size to determine statistical significance compared to other larger animal models. An athymic mouse subcutaneous model has been widely used for investigating the efficacy of cartilage regeneration in vivo with different biomaterials and cell types. Future work will involve testing this strategy in cartilage-defect models in large animals that better mimics the weight-bearing conditions in human. Harnessing synergistic interactions between stem cells and chondrocytes holds great promise for overcoming donor scarcity for repairing functional articular cartilage in patients across a broad range of demographic and age groups.

Methods

Cell Isolation and Expansion.

NChons and ADSCs were isolated as previously described (Lai et al. (2013) Sci Rep 3:3553). NChons were cryopreserved after isolation and were used in all experiments without further expansion. ADSCs were expanded for four passages in growth medium as defined in Supplementary Methods.

3D Hydrogel Co-Culture.

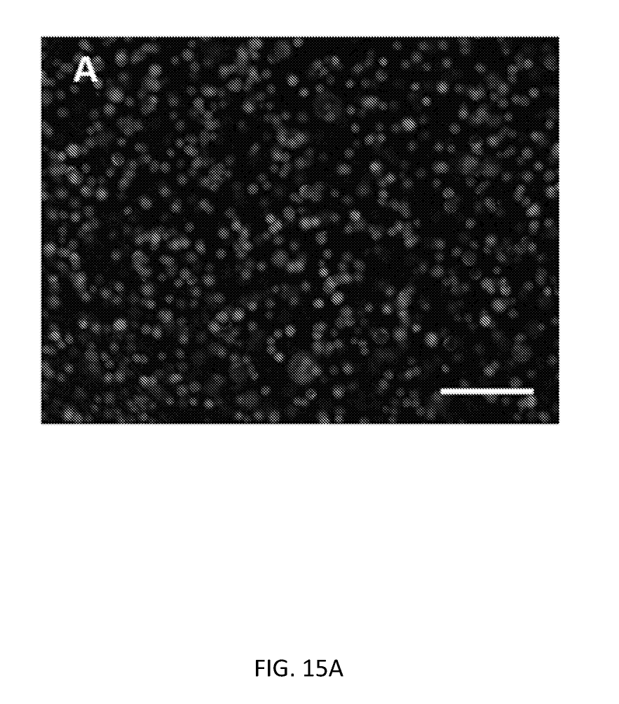

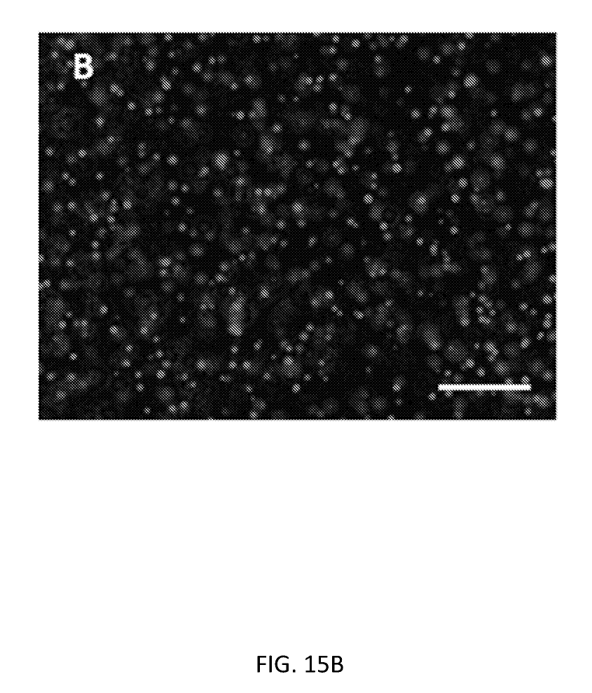

In all experiments, cells were suspended at 15×10⁶ cells/mL in a hydrogel solution consisting of 5% (w/v) poly(ethylene glycol diacrylate) (MW=5000 g/mol, Lysan Bio, Inc., Arab, Al), 3% (w/v) chondroitin sulfate-methacrylate, and 0.05% (w/v) photoinitiator (Irgacure D 2959, Ciba Specialty Chemicals, Tarrytown, N.Y.) in Dulbecco's phosphate-buffered saline. The cell-hydrogel suspension was pipetted into a custom-made cylindrical gel mold (50 μL volume) and exposed to light (365 nm at 3 mW/m² for 5 minutes) to induce cross-linking. Over 90% viability was observed 24 hours post-encapsulation for both ADSCs and NChons (FIG. 15). In all in vitro studies, cell-hydrogel constructs were either cultured in chondrogenic medium with or without 10 ng/ml of TGF-β3 (PeproTech, Rocky Hill, N.J.). Chondrogenic medium is defined in Supplementary Methods.

Varying the Ratio of Cell Types in Mixed Cell Populations.

To examine the effects of varying cell ratio on interaction synergy, we evaluated five ratios of mixed cells (NChon:ADSC 25:75, 10:90, 5:95, 2:98, and 1:99, resulting in NChon percentages of 25%, 10%, 5%, 2%, and 1%, respectively); pure NChon and ADSC populations seeded into hydrogels at the same cell density served as controls. All cell-hydrogel constructs were cultured for 21 days in CM. To assess the extent of interaction synergy and cartilage ECM production, biochemical analyses (n=3) and immunostaining (n=2) were carried out as described in Supplementary Methods.

Changing O₂ Tension and Culture Medium.

Mixed cell (25% NChons) and control (pure NChons or ADSCs) populations were cultured in 3D biomimetic hydrogels as described above for 14 days. Culture was carried out at 2% or 20% O₂ and chondrogenic medium with or without TGF-β3 supplementation. At the end of the 14-day in-vitro culture, biochemical evaluation (n=3) and immunostaining (n=2) were performed.

Subcutaneous Nude Mouse Model.

Animal studies were performed in accordance with the guidelines for the care and use of laboratory animals at Stanford University; all protocols were approved by the Stanford University Institutional Animal Care and Use Committee.

Cells were encapsulated in 3D biomimetic hydrogels and cultured in vitro for 2 weeks in chondrogenic medium with TGF-β3 supplementation prior to in vivo implantation in an athymic mouse subcutaneous model consisting of female nude mice (NCRNU, 9 weeks old; Taconic). Mixed cell populations of 25% or 10% NChons were chosen based on in-vitro results showing that mixed co-culture at these cell ratios led to optimal synergy and maximal cartilage ECM production (FIG. 10). Pure populations of ADSCs or NChons were included as controls. Biochemical and mechanical evaluations (n=5) as well as immunostaining (n=3) were carried out at weeks 3 and 8 after subcutaneous implantation (Supplementary Materials and Methods). Immunostaining was performed at week 12 after implantation (n=3).

Statistical Analysis.

GraphPad Prism 6 (GraphPad Software, San Diego, Calif., USA) was used for all statistical analyses. One- or two-way analysis of variance and pairwise comparisons with Tukey's post-hoc test were used to determine statistical significance (p<0.05). Data are represented as mean±standard deviation of at least three biological replicates.

Supplementary Materials

Cell Culture.

Human ADSCs were expanded in growth medium composed of high-glucose Dulbecco's Modified Eagle Medium supplemented with 5 ng/mL basic fibroblast growth factor, 10% fetal bovine serum (FBS), 100 U/mL penicillin and 0.1 mg/mL streptomycin (Invitrogen, Carlsbad, Calif.). Passage 4 ADSCs were used for the encapsulation. All cell-hydrogel constructs were cultured in chondrogenic medium with or without TGF-β3. Chondrogenic medium is consisted of high-glucose Dulbecco's Modified Eagle Medium (Invitrogen) containing 100 nM dexamethasone (Sigma-Aldrich, St. Louis, Mo., USA), 50 μg/mL ascorbate-2-phosphate (Sigma-Aldrich), 40 μg/mL proline (Sigma-Aldrich), 100 μg/mL sodium pyruvate (Invitrogen), 100 U/mL penicillin, 0.1 mg/mL streptomycin, and ITS Premix (5 μg/mL insulin, 5 μg/mL transferrin, 5 ng/mL selenious acid; BD Biosciences, San Jose, Calif.).

Biochemical Analyses.

At the time of harvest, cell-hydrogel constructs were weighed wet, lyophilized, weighed dry, and digested in papainase solution (Worthington Biochemical, Lakewood, N.J.) at 60° C. for 16 hours. DNA content was measured using the PicoGreen assay (Molecular Probes, Eugene, Oreg.) using Lambda phage DNA as a standard. sGAG content was quantified using the 1,9-dimethylmethylene blue dye-binding assay with shark chondroitin sulfate (Sigma-Aldrich, St. Louis, Mo.) as standard (Farndale et al. (1986) Acta 883(2):173-177). To determine GAG content contributed by the cells, we subtracted GAG content measured in the acellular hydrogels from the total GAG content from the cell-hydrogel constructs. Total collagen content was determined using acid hydrolysis followed by reaction with p-dimethylaminobenzaldehyde and chloramine T (Sigma-Aldrich). Collagen content was estimated by assuming a 1:7.46 hydroxyproline:collagen mass ratio (Stegemann & Stalder (1967) Clin Chim Acta 18(2):267-273). The interaction index, which is the measured matrix content (DNA, sGAG, or collagen) in the mixed co-culture group normalized by the expected matrix content based on the cell ratio and the measured matrix content per wet weight in the NChon and ADSC controls, was calculated (Acharya et al. (2012) J Cell Physiol 227(1):88-97). An interaction index greater than 1 indicates that the resulting matrix content is higher than expected.

Mechanical Testing.

Unconfined compression tests were conducted using an Instron 5944 Materials Testing System (Instron Corporation, Norwood, Mass., USA) fitted with a 10-N load cell (Interface Inc., Scottsdale, Ariz., USA). During testing, cell-hydrogel constructs were submerged in a bath of phosphate-buffered saline at room temperature. Constructs were compressed at a rate of 1% strain/s to a maximum strain of 30% strain/s. Stress versus strain curves were created, and curves were fit using a third-order polynomial. The compressive tangent modulus was determined from the curve-fit equation at strain values of 10-20% strain/s.

Histology and Immunostaining.

Cell-hydrogel constructs were fixed in 4% paraformaldehyde (Sigma-Aldrich) overnight and stored in 70% ethanol at 4° C. until processed. Constructs were then embedded in paraffin and processed using standard histological procedures.

For immunostaining, enzymatic antigen retrieval was performed via incubation in 0.1% trypsin (Invitrogen, Carlsbad, Calif., USA) at 37° C. for 15 minutes. Sections were then blocked with blocking buffer consisting of 2% goat serum (Invitrogen), 3% bovine serum albumin (Fisher Scientific, Pittsburgh, Pa., USA), and 0.1% Triton X-100 (Sigma-Aldrich) in 1× phosphate-buffered saline, followed by incubation in rabbit polyclonal antibody against type I collagen, type II collagen, or aggrecan (1:100, Abcam, Cambridge, Mass., USA) overnight at 4° C. and incubation with secondary antibody (1:200, Alexa Fluor 488 goat anti-rabbit, Invitrogen) for 1 hour at room temperature. Nuclei were counterstained with 4′,6-diamidino-2-phenylindole mounting medium (Vectashield, Vector Laboratories, Burlingame, Calif., USA). Images were taken with a Zeiss fluorescence microscope.

Cell Tracking Using Membrane Labeling.

To track cell distributions within the hydrogel constructs over time, ADSCs were labeled with red fluorescent dye (PKH26, Sigma-Aldrich) at a concentration of 4 μM for 4 minutes according to the manufacturer's protocol prior to encapsulation. Labeled ADSCs were encapsulated with NChons in the mixed co-culture hydrogel model at various cell ratios for 21 days (n=3 for each ratio). Samples were fixed in 4% paraformaldehyde overnight, submerged in 30% sucrose (Sigma-Aldrich) solution for 24 hours, embedded in Tissue-Tek (Sakura Finetek, Torrance, Calif.), and frozen in liquid nitrogen. Cryosections (12 μm thick) were washed in Dulbecco's phosphate-buffered saline, and type II collagen and cell nuclei were stained using the immunostaining procedures described above.

### Example 3

### Modulating Stem Cell-Chondrocytes Interactions for Cartilage Repair Using Combinatorial Extracellular Matrix-Containing Hydrogels

1 Introduction

Osteoarthritis is one of the most common joint diseases in the world and causes a loss in quality of life. Cartilage is avascular and has little ability to self-repair and to regenerate once damaged. Current cell-based therapies for cartilage repair involve the use of autologous chondrocytes, which are associated with disadvantages including donor-site morbidity, limited availability, and de-differentiation during expansion (Tuan et al. (2007) Arthritis Res Ther 9:109). Therefore, there is a strong need for alternative cell sources to reduce the number of chondrocytes needed for cartilage repair. Stem Cells such as bone marrow-derived mesenchymal stem cells or adipose-derived stem cells (ADSCs) are attractive autologous cell sources for cartilage repair given their chondrogenic potential. However, stem cells generally do not produce significant amounts of cartilage-specific matrix, making clinical translation of stem cells for cartilage regeneration challenging (Wang et al. (2014) Tissue Engineering Part A 20:2131-2139; Erickson et al. (2009) Tissue Engineering Part A 15:1041-1052). Co-culturing stem cells with primary chondrocytes constitutes a viable solution to reduce the number of chondrocytes needed while simultaneously increasing the production of cartilage-specific matrix (Meretoja et al. (2012) Biomaterials. 33:6362-69; Lai et al. (2013) Scientific Reports 3:3553; Acharya et al. (2012) J Cell Physiol 227:88-97; Bian et al. (2011) Tissue Engineering Part A 17:1137-1145; Wu et al. (2011) Tissue Engineering Part A 17:1425-1436).

In a co-culture system, stem cells interact with chondrocytes via paracrine signaling and can lead to enhanced cartilage matrix deposition. Most previous co-culture studies utilize bone marrow mesenchymal stem cells and aim to employ chondrocyte to enhance the chondrogenesis of stem cells (Lai et al. (2013) Scientific Reports 3:3553; Liu et al. (2010) Biomaterials 31:9406-14). Different than conventional approach, we have recently reported that adipose-derived stem cells, a more abundantly available cell source can substantially increase the cartilage forming capacity of juvenile chondrocytes when mixed co-cultured in 3D hydrogels. ADSCs are particularly attractive given their ease of isolation and abundance from liposuction (Estes et al. (2010) Nature Protocols 5:1294-311; Awad et al. (2004) Biomaterials 25:3211-3222). These discrepancies in findings may be due to variations in culture platforms, growth-factor supplementation, or chondrocyte phenotype (Meretoja et al. (2012) Biomaterials 33:6362-69; Wu et al. (2011) Tissue Engineering Part A 17:1425-1436; Qing et al. (2011) 44:303-310; Lee et al. (2012) Stem Cell Res Ther 3:35; Xu et al. (2013) Stem Cells Dev 22:1657-1669; Giovannini et al. (2010) Eur Cell Mater 20:245-259). In particular, some groups performed co-culture experiments using cell pellets while others encapsulated cells within synthetic hydrogels, such as poly(ε-caprolactone) or polylactic acid/polyglycolic acid scaffolds, or natural hydrogels such as hyaluronic acid, fibrin, or alginate-based hydrogels (Meretoja et al. (2012) Biomaterials 33:6362-6369; Acharya et al. (2012) J Cell Physiol 227:88-97; Bian et al. (2011) Tissue engineering Part A 17:1137-1145; Liu et al. (2010) Biomaterials 31:9406-9414; Leyh et al. (2014) Stem Cell Res Ther 5:77; Mo et al. (2009) Bone 45:42-51). The influences of scaffold type on the interactions between stem cells and chondrocytes have not been well studied.

Here, we hypothesized that the biochemical and mechanical properties of the material platform direct stem cell-chondrocyte interactions, thereby affecting the overall outcome of cartilage-specific matrix accumulation by cells. We previously demonstrated that biochemical cues provided by methacrylated extracellular matrix (ECM) molecules in hydrogels, as well as the mechanical properties of hydrogels, impact the chondrogenic gene expression of ADSCs in 3D culture (Wang et al. (2014) Tissue Engineering Part A 20:2131-2139). These changes may result in changes in paracrine signaling that may in turn direct chondrocytes to secrete different amounts of cartilage-specific matrix. Therefore, we systematically investigated the effects of materials on modulating the interactions between ADSCs and bovine neonatal chondrocytes (NChons) using a biomimetic 3D hydrogel platform containing the cartilage-specific ECM molecules chondroitin sulfate methacrylate (CS-MA), hyaluronic acid methacrylate (HA-MA), and heparan sulfate methacrylate (HS-MA). These biochemical cues were incorporated homogenously into the bulk poly-(ethylene glycerol) dimethacrylate (PEGDMA) (4.6 kDa) hydrogel at four concentrations (0.5%, 1.25%, 2.5% and 5% (w/v)) via photocrosslinking. To study how mechanical cues influence ADSC-chondrocyte interactions, we selected three values of mechanical stiffness (15 kPa, 8% (w/v); 40 kPa, 11% (w/v); 100 kPa, 14% (w/v)) to represent soft, moderate, and stiff matrices. Taken together, these investigations indicated that decoupling biochemical and mechanical properties in a synthetic 3D cell niche yielded insights into the interactions between ADSCs and NChons that could be harnessed for clinical cartilage repair.

2 Experimental

2.1 Synthesis of Methacrylated Extracellular Matrix Molecules

Unless otherwise stated, all chemicals used in the methacrylation of ECM molecules were purchased from Sigma.

CS-MA was synthesized by modification of a previously reported method (Jeon et al. (2009) Biomaterials 30:2724-2734; Baier et al. (2003) Biotechnol Bioeng 82:578-589). Briefly, chondroitin sulfate sodium salt was reacted with N-hydroxysuccinimide and 1-ethyl-3-(3-dimethylaminopropyl)-carbodiimide for 5 minutes in an MES buffer before the addition of 2-aminoethyl methacrylate at a molar ratio of 1:2:1. These compounds were reacted for 24 hours at room temperature, dialyzed, lyophilized, and stored at −20° C. until use. HS-MA was synthesized using heparan sulfate sodium salt following the same protocol.

HA-MA was synthesized through a modification of previously reported methods (Baier et al., supra; Suri et al. (2010) Tissue Engineering Part A 16:1703-1716). Briefly, triethylamine and glycidyl methacrylate was added to 20 k MW sodium hyaluronate (Lifecore) and reacted at room temperature for 24 hours before acetone precipitation. The precipitate was then dissolved, dialyzed, lyophilized, and stored at −20° C. until use.

In this study, methacrylated ECM molecules were synthesized with less methacrylation reagents, resulting in fewer methacrylate groups on each ECM molecule. Details of the synthesis of each ECM molecule appear in Supplementary Table 51.

2.2 Cell Isolation and Culture

NChons were obtained from the dissection of hyaline articular cartilage from the femoropatellar groove of the stifle joints from a four-day-old calf (Research 87 Inc.). The dissected cartilage was first washed in Dulbecco's phosphate-buffered saline and then further dissected into small pieces before being placed in Dulbecco's Modified Eagle Medium (Gibco, Invitrogen) supplemented with 1 mg/mL collagenase type II and type IV (Worthington Biochemical) for digestion. After 24 hours at 37° C., the cell suspension was filtered through a 70-μm cell strainer, washed with Dulbecco's phosphate-buffered saline, and centrifuged. Cells were counted, frozen, and stored in liquid nitrogen as passage 0 NChons.

Human ADSCs were isolated from human adipose tissue using the method described by Zuk et al. (Tissue Engineering (2001) 7:211-228). ADSCs were then cultured in high-glucose Dulbecco's Modified Eagle Medium supplemented with 5 ng/mL basic fibroblast growth factor (PeproTech), 10% (v/v) fetal bovine serum (Gibco, Invitrogen), 100 U/mL penicillin (Gibco, Invitrogen), and 0.1 mg/mL streptomycin (Gibco, Invitrogen). ADSCs were expanded for four passages before use as passage-5 ADSCs.

2.3 Cell Viability

To ensure that our hydrogels were non-toxic to NChons, we cultured NChons in hydrogels containing 11% (w/v) PEGDMA and CS-MA, HA-MA, or HS-MA at 0%, 0.5%, and 5% (w/v). NChons were harvested from these hydrogels 24 hours and 14 days after encapsulation to access the short- and long-term effects of hydrogel composition on cell viability using the LIVE/DEAD Cell Viability Assay kit (Life Technologies) in accordance with the manufacturer's protocol. A thin slice sectioned from each hydrogel was immersed into assay reagent solution for 30 minutes before imaging using a Zeiss fluorescence microscope.

2.3 Hydrogel Formation

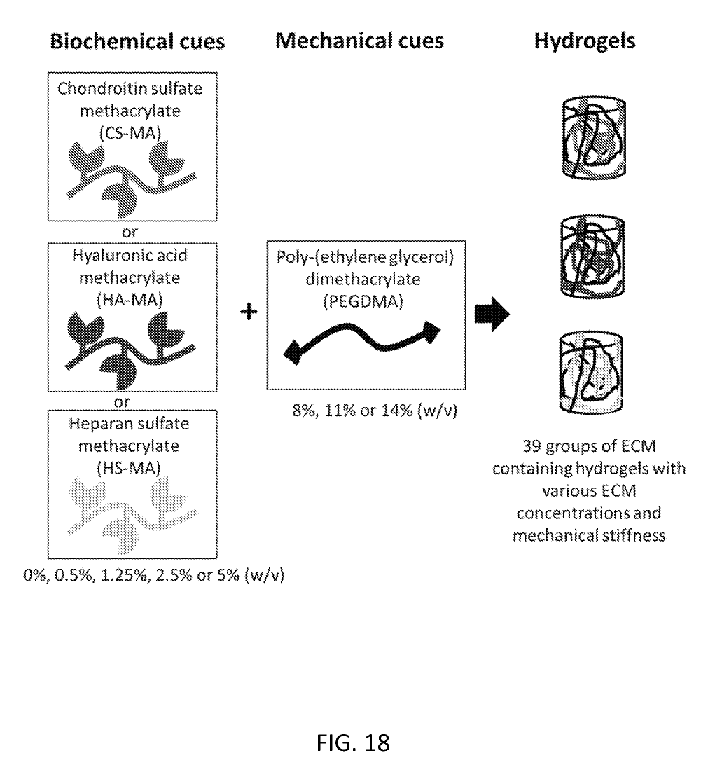

A total of 39 hydrogel types containing varying ECM compositions, ECM molecule concentrations, and mechanical stiffness were used in this study (FIG. 18). To vary mechanical stiffness, PEGDMA (MW 4.6 kDa) was dissolved in sterile Dulbecco's PBS (Gibco, Invitrogen) to achieve final concentrations of 8%, 11%, and 14% (w/v). To tune the biochemical composition of the hydrogels, various methacrylated ECM molecules, CS-MA, HA-MA, and HS-MA were added at 0.5%, 1.25%, 2.5% and 5% (w/v). Control hydrogels are made with PEGDMA only (no ECM molecules were added). All hydrogel precursor solutions contained 0.05% (w/v) lithium phenyl-2,4,6-trimethylbenzoylphosphinate as a photoinitiator.

2.4 Cell Encapsulation and Culture

On the day of encapsulation, passage-4 ADSCs were trypsinized and counted. NChons were thawed and counted without further expansion. Cells were mixed in a ADSC:NChon ratio of 3:1 and homogenously suspended in the hydrogel precursor solution at 1.5×10⁷ cells/mL. The cell-hydrogel precursor solution was pipetted into a 96-well mold (50 μL per gel) and exposed to ultraviolet light (365 nm) for 3 minutes at 4 mW/m² for photocrosslinking.

All samples were cultured in 1.5 mL of chondrogenic medium composed of high-glucose Dulbecco's Modified Eagle Medium containing 100 nM dexamethasone (Sigma-Aldrich), 50 mg/mL ascorbate-2-phosphate (Sigma-Aldrich), 40 mg/mL proline (Sigma-Aldrich), 100 mg/mL sodium pyruvate (Gibco, Invitrogen), 100 U/mL penicillin, 0.1 mg/mL streptomycin, and 5 μg/mL ITS Premix (BD Biosciences) supplemented with 10 ng/mL TGF-β3 (PeproTech) for 3 weeks; medium was exchanged every other day.

2.4 Mechanical Testing

Unconfined compression tests were conducted using an Instron 5944 materials testing system (Instron Corporation) fitted with a 10-N load cell (Interface Inc.). Our set-up consisted of custom-made aluminum compression platens lined with PTFE to minimize friction. Specimen diameter and thickness were measured using digital calipers and the material testing system's position read-out, respectively. A 2-mN preload was applied before each test and the upper plate was lowered at a rate of 1% strain/s. The compressive modulus was determined from 10-20% of the linear curve fit from the stress versus strain curve. The mechanical stiffness of acellular hydrogels and cell-laden hydrogels on day 21 of culture was measured. All tests were conducted in phosphate-buffered saline at room temperature.

2.5 Biochemical Assays

After 3 weeks of culture, cell-laden hydrogels were harvested and their wet weights were measured. The hydrogels were frozen, lyophilized, and the dry weights of each hydrogel were determined. The lyophilized hydrogels were each digested in 500 μL of papainase solution (Worthington Biochemical) at 60° C. for 16 hours. Supernatants were collected for subsequent biochemical assays. At least three replicate hydrogels were used for each biochemical assay.

DNA content was measured using the PicoGreen assay kit (Molecular Probes) in accordance with the manufacturer's protocol, using lambda phage DNA as standard. Sulfated glycosaminoglycan (sGAG) content was quantified spectrophotometrically using the 1,9-dimethylmethylene blue dye-binding assay (pH 3.0). Shark chondroitin sulfate (Sigma) was used as the standard. Hydroxyproline content was determined using Ehrlich's reaction assay as previously described (Stegemann et al. (1967) Clin Chim Acta 18:267-273). Briefly, concentrated hydrochloric acid was added to 50 μL of supernatant (from lyophilized cell-laden hydrogel) and acid hydrolysis was carried out at 110° C. for 16 hours. Samples were dried under a sodium hydroxide ice-trap under vacuum conditions. Dried samples were reconstituted in water and reacted with p-dimethylaminobenzaldehyde and chloramine T (Sigma). After a 20-minute incubation at 60° C., the absorbance of each sample was read at 540 nm and compared to a hydroxyproline standard. Collagen content was estimated by assuming a mass ratio of 1:7.46 hydroxyproline collagen mass (Estes et al. (2010) Nature Protocols 5:1294-1311; Stegemann et al. (1967) Clin Chim Acta 18:267-273).

2.6 Histology

Cellular hydrogels were harvested after 3 weeks of culture, fixed in 4% (w/v) paraformaldehyde (Sigma) overnight at 4° C., and immersed in a 30% (w/v) sucrose solution overnight at 4° C. Samples were then snap-frozen in Optimal Cutting Temperature solution and stored at −80° C. Cryosectioning was performed at −20° C.

For immunostaining, enzymatic antigen retrieval was performed by incubating sections with 0.1% trypsin (Gibco) at 37° C. for 15 minutes. Sections were blocked with 2% goat serum (Gibco, Invitrogen) in 3% (w/v) bovine serum albumin (Fisher Scientific) solution for 1 hour at room temperature. For primary antibodies, rabbit polyclonal antibody against collagen type I, II, or X (Abcam) or against aggrecan (kind gift from Prof. R L Smith) was added to the sections and incubated overnight at 4° C. For secondary antibody, Alexa Fluor 488 goat anti-rabbit (Invitrogen) was added to the sections and incubated for 1 hour at room temperature. Cell nuclei were counterstained with Hoechst dye 33342 (Cell Signaling Technologies) for 1 hour at room temperature. Sections were then mounted with VECTASHIELD (Vector Laboratories) and imaged with a Zeiss fluorescence microscope.

2.7 Statistical Analysis

All experiments are performed with at least three replicates. GraphPad Prism (Graphpad Software) was used for statistical analyses. Statistical significance was determined using one- or two-way analysis of variance and pairwise comparisons with Tukey's post-hoc test (p<0.05).

3 Results

3.1 Largely Decoupled Biochemical and Mechanical Properties of Combinatorial Hydrogels

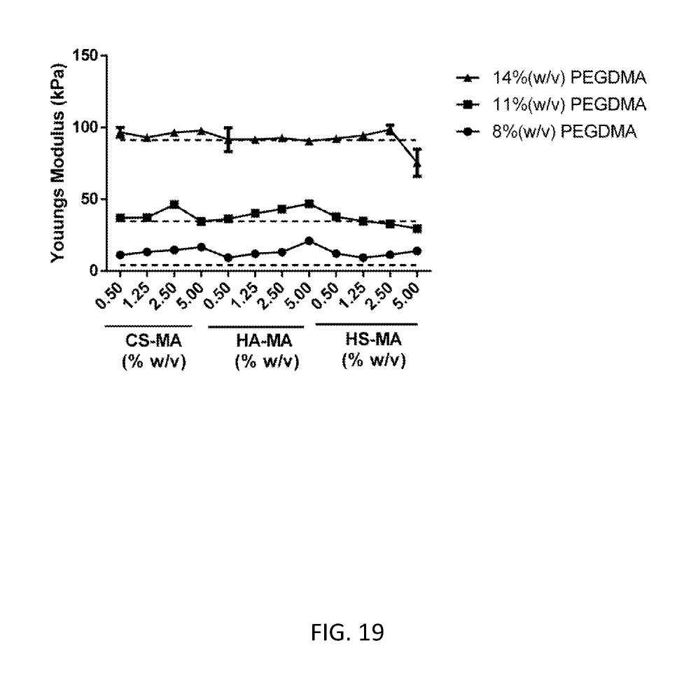

Unconfined compression testing was performed on our combinatorial hydrogels to measure their Young's moduli. Three distinct values of mechanical stiffness (˜15 kPa, ˜40 kPa, and ˜100 kPa) were obtained by varying PEGDMA concentration (8%, 11%, and 14% (w/v), respectively; FIG. 19). The incorporation of ECM molecules of up to 5% (w/v) had minimal effects on mechanical stiffness in 11% and 14% (w/v) hydrogels (FIG. 2). In contrast, incorporating methacrylated ECM molecules into hydrogels containing 8% (w/v) PEGDMA increased the mechanical stiffness of all hydrogels to a similar extent (FIG. 19).

3.2 Modulation of ADSC-NChon Interactions in Combinatorial Hydrogels

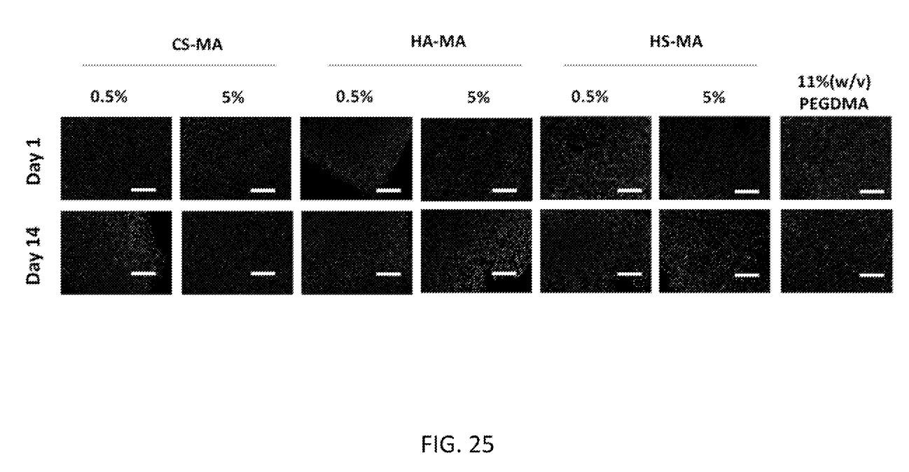

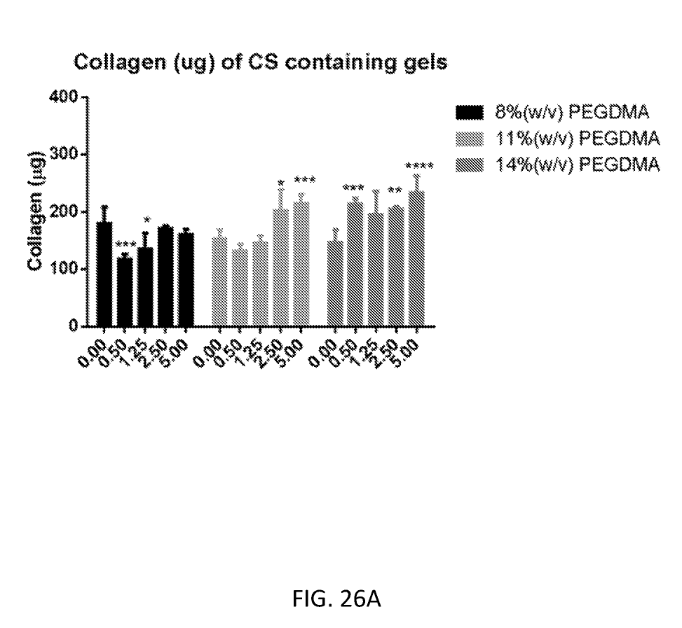

Cell viability was assayed at 24 hours after NChons were encapsulated in hydrogels and again 14 days after encapsulation in hydrogels containing 11% (w/v) PEGDMA (FIG. 25). Cell viability was high (>90%) across all hydrogel compositions examined (FIG. 25).

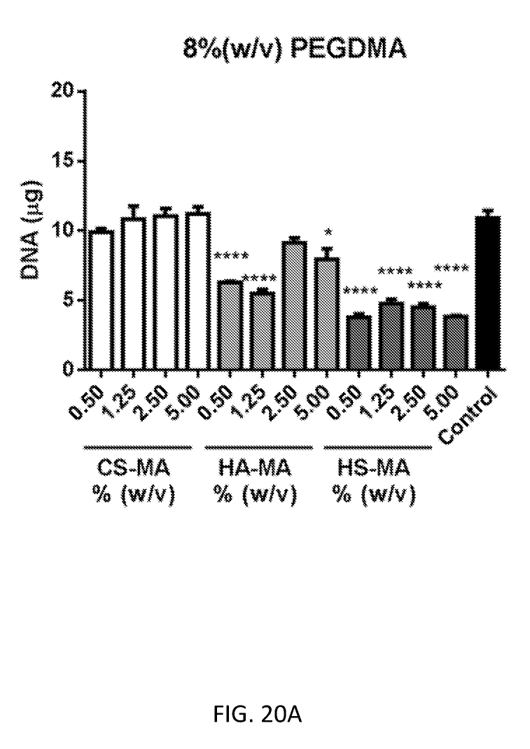

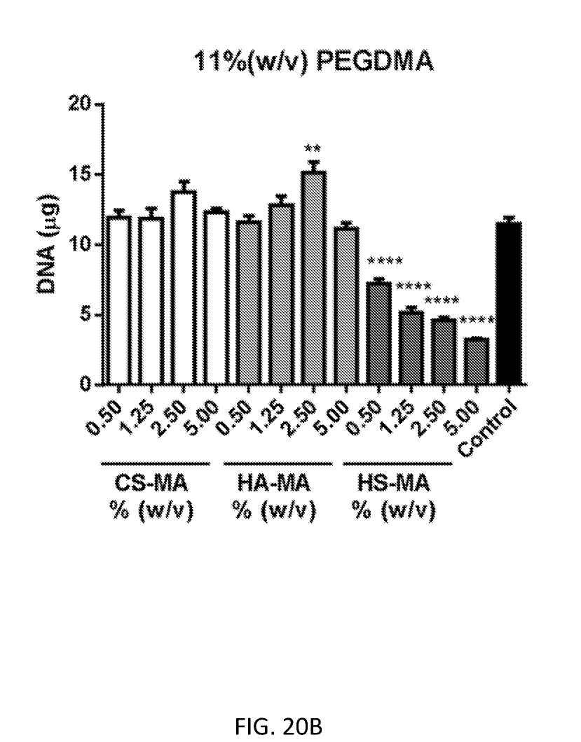

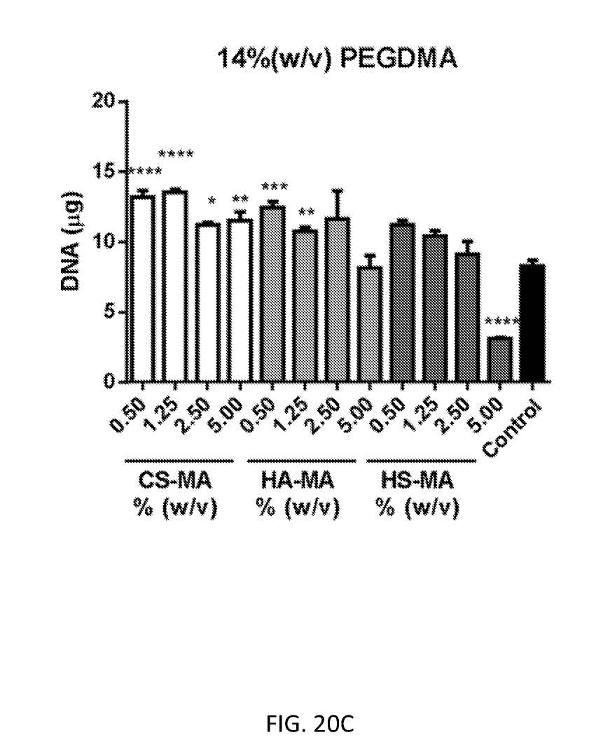

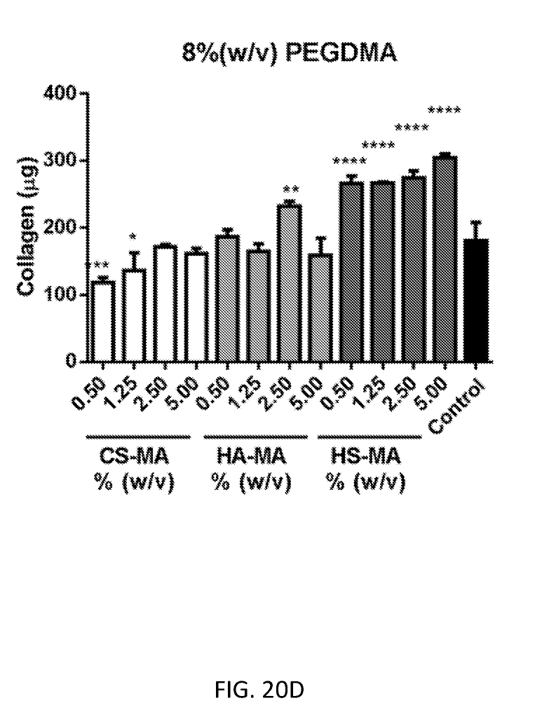

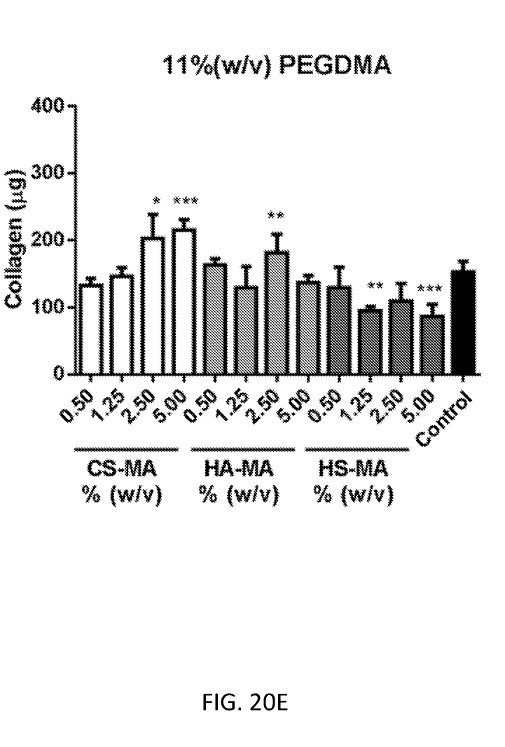

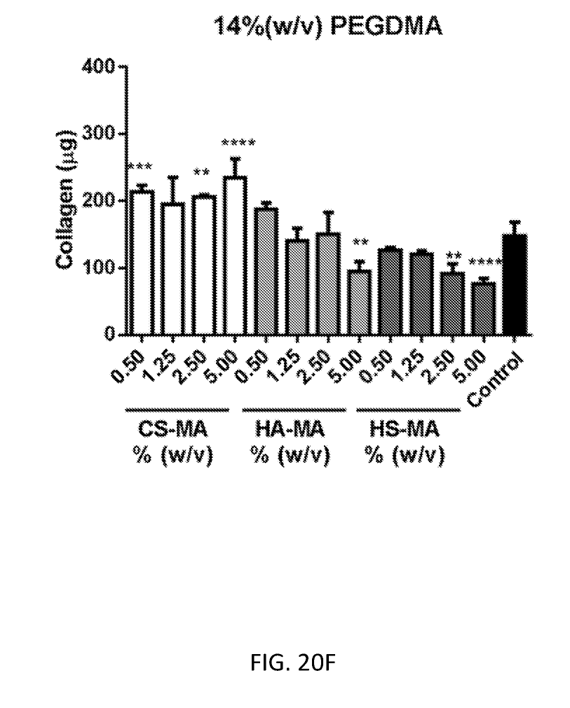

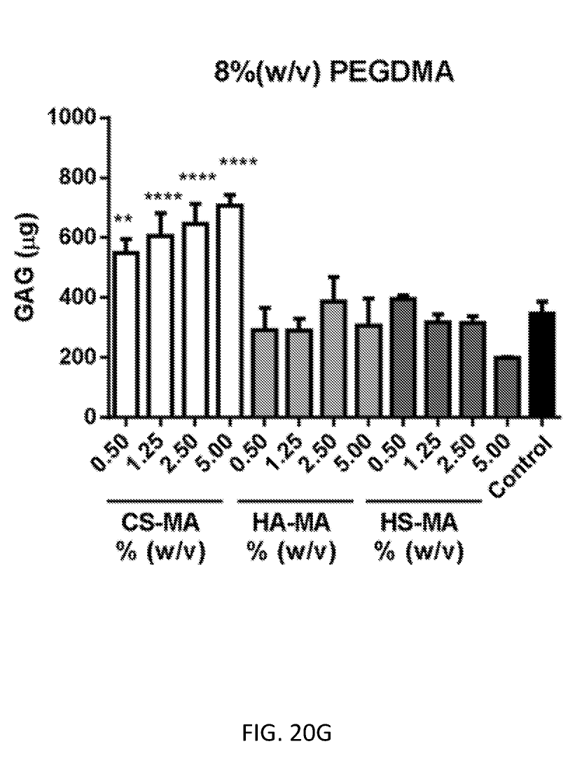

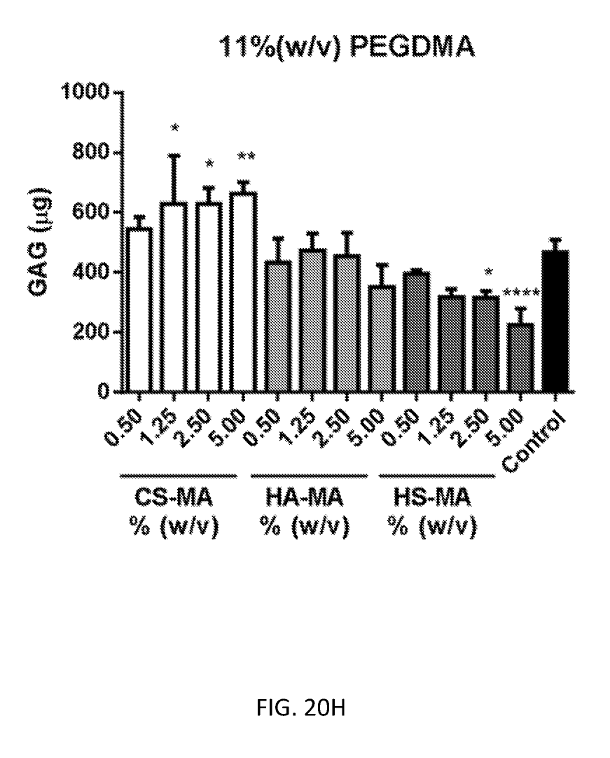

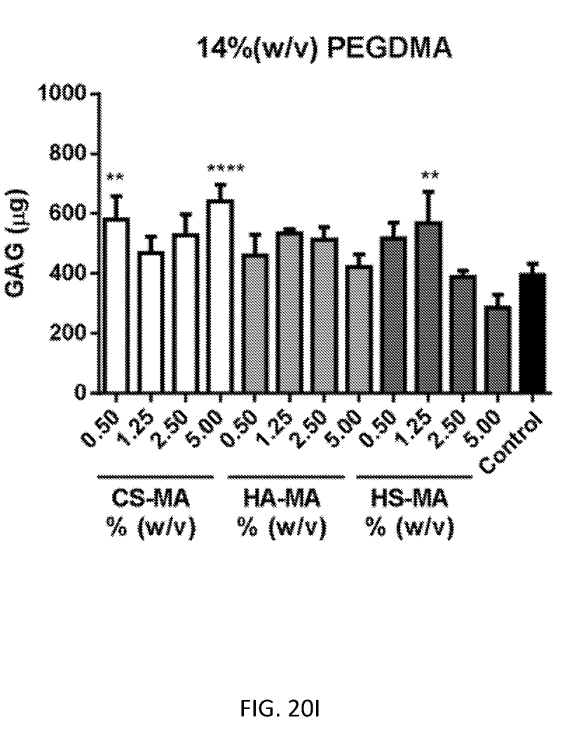

Quantification of the amount of DNA in each hydrogel after 21 days of culture under chondrogenic conditions revealed that mechanical stiffness only had mild effects on day 21 (FIGS. 20A-20C), whereas tuning the concentrations of methacrylated ECM components in the hydrogel significantly affected DNA content (FIGS. 20A-20C). Across all stiffness values tested, CS-MA-containing hydrogels yielded the highest amount of DNA (FIGS. 20A-20C). HA-MA supported less cell proliferation than CS-MA in softer hydrogels (FIG. 20A), but in hydrogels of higher mechanical stiffness (FIGS. 20B-20C) HA-MA supported cell proliferation to approximately the same extent as CS-MA. A high concentration of HS-MA appeared to inhibit cell proliferation in hydrogels containing 11% and 14% (w/v) PEGDMA versus CS-MA and HA-MA (FIGS. 20B-20C). Control hydrogels lacking ECM molecules more effectively promoted cell proliferation at lower mechanical stiffness (8% and 11% (w/v) PEGDMA; FIGS. 20A-20B). Increasing the PEGDMA concentration to 14% (w/v) resulted in decreases in cell density by 24% and 28% versus 8% and 11% (w/v) PEGDMA hydrogels, respectively (FIG. 20C).

To better understand how biochemical and mechanical properties modulate the synergistic interactions between ADSCs and NChons in 3D, we evaluated the production of sulfated glycosaminoglycan (sGAG) and collagen in each of the 39 groups of our combinatorial hydrogels (FIG. 18). The effects of biochemical cues on cartilage matrix production were dependent on the mechanical stiffness of the hydrogel (FIGS. 20D-20I). In softer hydrogels (8% (w/v) PEGDMA), HS-MA promoted the highest amount of collagen secretion, while cells encapsulated in CS-MA-containing hydrogels secreted the least collagen (FIG. 20D). At this mechanical stiffness, cells encapsulated in hydrogels containing 5% (w/v) HS-MA produced 88% more collagen matrix than cells encapsulated in hydrogels containing 5% (w/v) CS-MA (FIG. 20D). However, in hydrogels of moderate mechanical stiffness (11% (w/v) PEGDMA), this trend was reversed (FIG. 20E). CS-MA-containing hydrogels of moderate mechanical stiffness harbored cells that secreted more collagen than did HS-MA containing hydrogels, and a positive dose dependence was evident when the concentration of CS-MA was increased from 0.5% to 5% (w/v; a 62% increase in collagen; FIG. 20E). In the stiffest hydrogels (14% (w/v) PEGDMA), CS-MA prompted the most collagen production (FIG. 20F). In contrast, both HA-MA and HS-MA elicited a negative dose response, with collagen production falling by 49% and 40%, respectively, as the concentrations of the ECM molecule was increased from 0.5% to 5% (w/v) (FIG. 20F). HS-MA was least efficient in modulating synergistic matrix production in stiff hydrogels (FIG. 20F).

The sGAG production displayed trends that were opposite the trends in collagen production in soft hydrogels (8% (w/v) PEGDMA) (FIG. 20G). The presence of only 0.5% (w/v) CS-MA yielded 58% more sGAG secretion than controls in the 8% PEGDMA hydrogels (FIG. 20G). In addition, when the concentration of CS-MA increased, a positive dose response was observed, with sGAG production increasing by 29% as the CS-MA concentration increased from 0.5% to 5% (w/v) (FIG. 20G). However, unlike collagen production, sGAG production was halved as the HS-MA concentration within the 8% PEGDMA hydrogel rose from 0.5% to 5% (w/v) (FIG. 20G). This trend was also evident at the moderate mechanical stiffness of 11% (w/v) PEGDMA hydrogels (FIG. 20H). In 14% (w/v) PEGDMA hydrogels (˜100 kPa), the presence of CS-MA or HA-MA resulted in comparable sGAG production by the encapsulated cells (FIG. 20I). Although HS-MA still prompted a negative dose response in stiff hydrogels, sGAG production by cells in HS-MA-containing hydrogels was higher in these hydrogels than in hydrogels with moderate or soft mechanical stiffness (FIG. 20I).

3.3 Mechanical Stiffness of Cell-Hydrogel Constructs after 21 Days

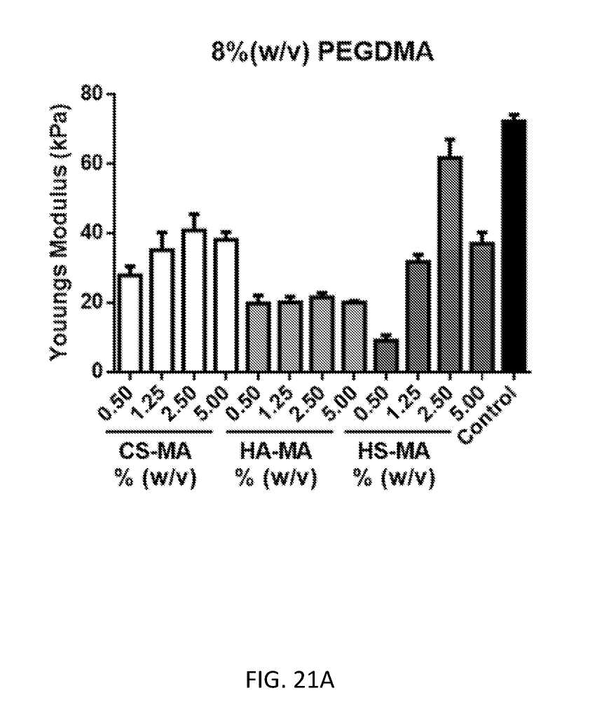

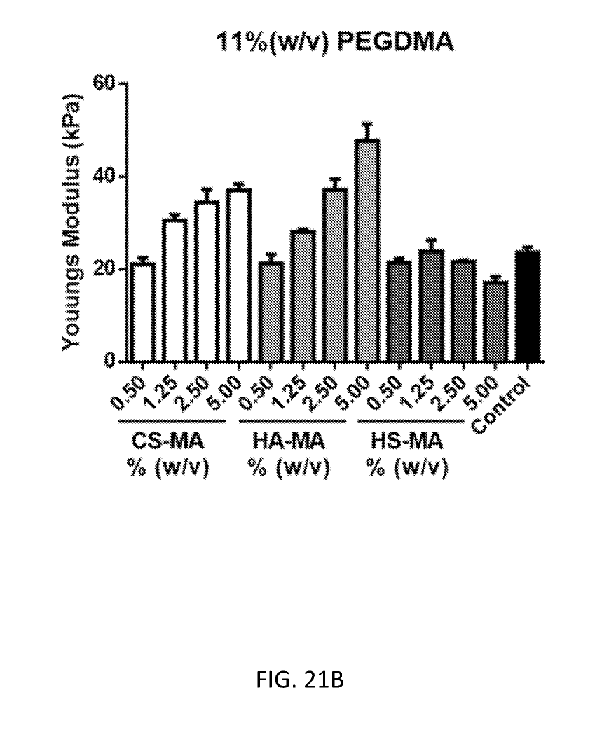

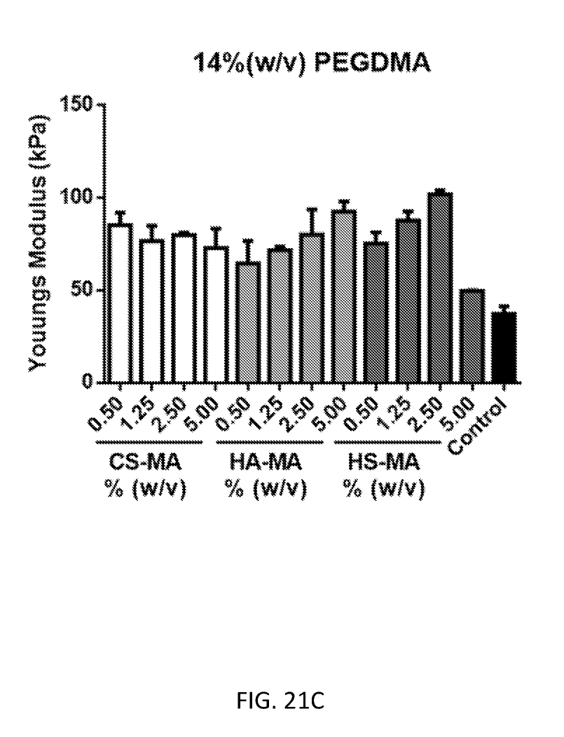

Unconfined compression testing was carried out on cell-laden combinatorial hydrogels after 21 days of culture under chondrogenic conditions. Hydrogels containing 8% (w/v) PEGDMA had higher mechanical stiffness than acellular hydrogels (FIG. 21A), while hydrogels containing 11% (w/v) PEGDMA (FIG. 21B) or 14% (w/v) PEGDMA (FIG. 21C) had slightly decreased mechanical stiffness compared to acellular hydrogels.

In CS-MA-containing 8% (w/v) PEGDMA hydrogels in a CS-MA concentration-dependent manner (FIG. 21A). Hydrogels containing 0.5% (w/v) CS-MA were slightly stiffer after 21 days in culture (˜28 kPa vs. ˜15 kPa) whereas hydrogels containing 5% (w/v) CS-MA measured at ˜38 kPa after 21 days in culture (FIG. 21A). The mechanical stiffness of HA-MA-containing hydrogels increased slightly after 21 days (FIG. 21A). Including HS-MA in the hydrogels resulted in a dose-dependent increase in mechanical stiffness at lower HS-MA concentrations (FIG. 21A). However, 5% (w/v) HS-MA did not increase hydrogel mechanical stiffness after 21 days (FIG. 21A).

Cell-laden hydrogels containing 11% (w/v) PEGDMA and CS-MA or HA-MA became softer in a dose-dependent manner after 21 days at low doses (0.5%, 1.25%, and 2.5% (w/v)), while hydrogels containing 5% (w/v) CS-MA or HA-MA maintained their original mechanical stiffness of ˜40 kPa (FIG. 21B). At 0.5% (w/v) CS-MA or HA-MA, cell-laden hydrogels measured at ˜20 kPa (FIG. 21B). HS-MA-containing hydrogels all softened to ˜20 kPa after 21 days of culture (FIG. 21B).

A small decrease in mechanical stiffness was observed across all hydrogels containing 14% (w/v) PEGDMA (FIG. 21C). The largest decrease in mechanical stiffness was evident in 5% (w/v) HS-MA hydrogels; the Young's modulus dropped to 49 kPa after 21 days in culture (FIG. 21C). In these stiff hydrogels, 5% (w/v) HA-MA partially rescued the drop in mechanical stiffness (FIG. 21C).

Control hydrogels did not contain any methacrylated ECM molecules. Of these hydrogels, those with 8% (w/v) PEGDMA had the highest increase in mechanical stiffness (an average of 72 kPa) after 21 days (FIG. 21A). A small decrease in mechanical stiffness occurred in 11% (w/v) PEGDMA control hydrogels (to 23 kPa; FIG. 21B) and a large decrease in mechanical stiffness was detected in 14% (w/v) PEGDMA control hydrogels (to 36 kPa; FIG. 21C).

3.4 Immunostaining of Cartilage-Specific Biomarkers

Significant differences in collagen (FIGS. 20D-20F) and sGAG (FIGS. 210G-20I) production were observed between hydrogels of varying mechanical stiffness containing 5% (w/v) ECM. To investigate the spatial organization of this newly deposited cartilage matrix, hydrogels containing 5% (w/v) CS-MA, HA-MA, or HS-MA were cryosectioned and immunostained for aggrecan as well as collagens I, II, and X.

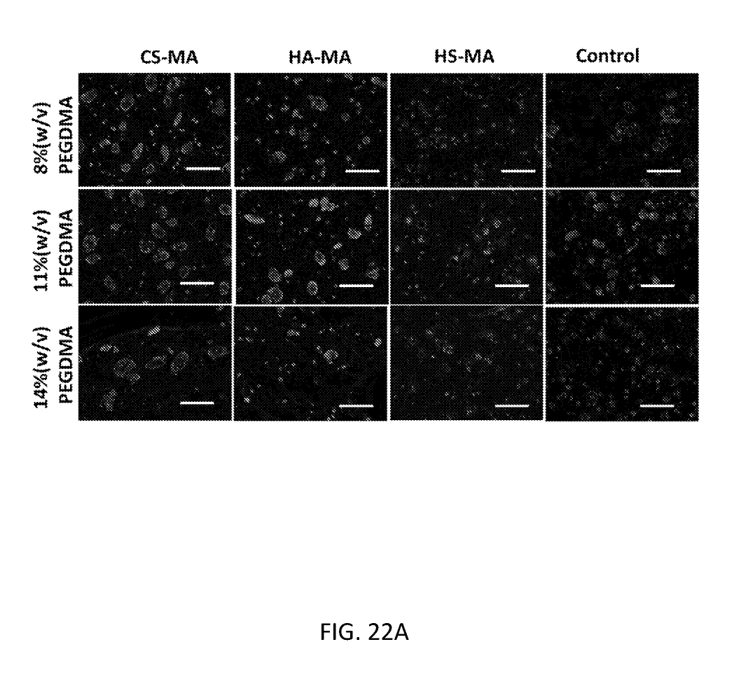

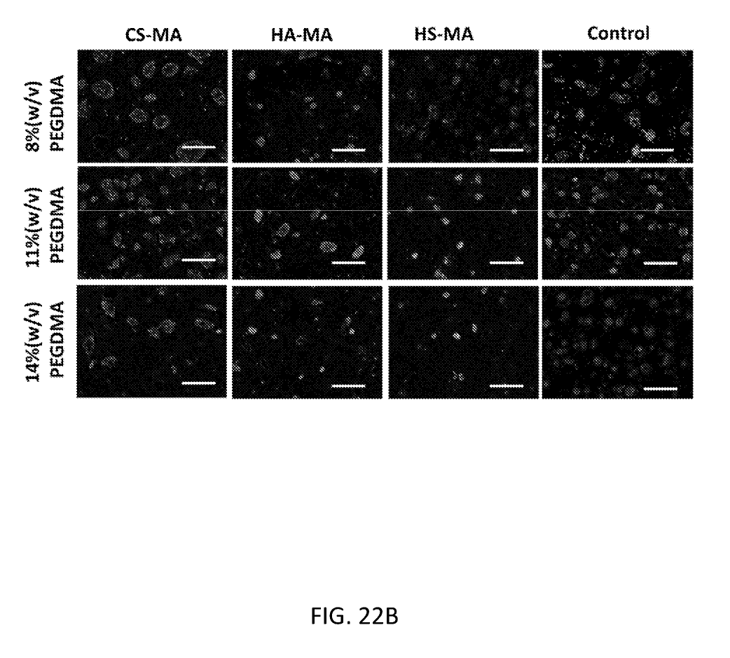

Large and well-defined collagen II and aggrecan nodules were observed in hydrogels containing 5% (w/v) CS-MA and 5% (w/v) HA-MA across all values of mechanical stiffness tested (FIG. 22). In addition, aggrecan nodules produced by cells in 5% (w/v) CS-MA-containing hydrogels were larger but stained less intensely for collagen type II (FIG. 22A) and aggrecan (FIG. 22B) than nodules in 5% (w/v) HA-MA-containing hydrogels. Cells in 5% (w/v) HS-MA-containing hydrogels produced more diffuse collagen II nodules (FIG. 22A) and smaller aggrecan nodules (FIG. 22B). In control hydrogels lacking ECM, collagen II nodules decreased in size as the amount of PEGDMA increased (FIG. 22A), but aggrecan nodules did not (FIG. 22B).

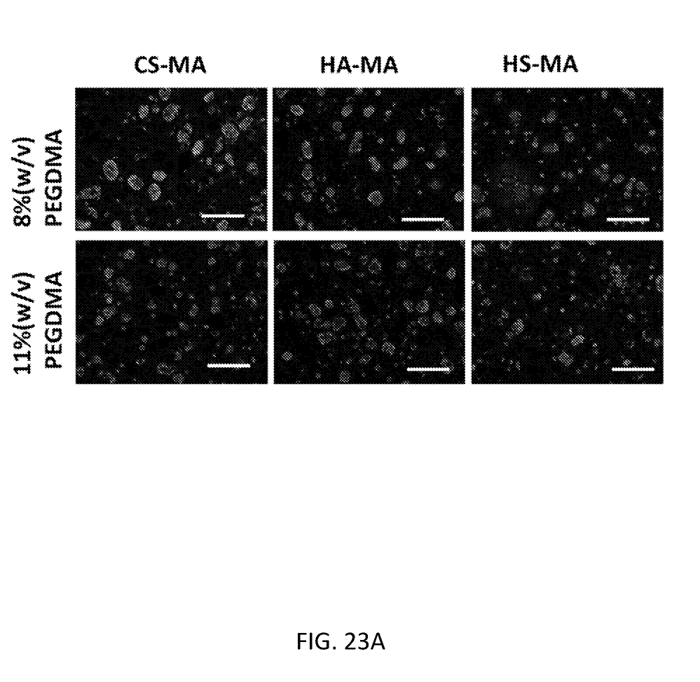

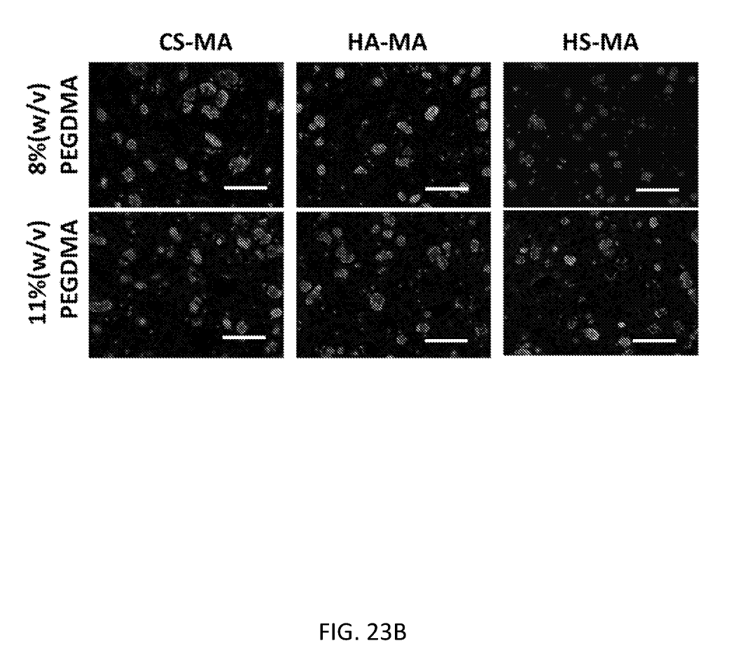

A low concentration of ECM (0.5%) was sufficient to result in the formation of collagen II and aggrecan nodules (FIG. 23). Similar to the nodules in hydrogels containing 5% (w/v) methacrylated ECM, collagen II (FIG. 23A) and aggrecan (FIG. 23B) nodules in 0.5% (w/v) CS-MA and 0.5% (w/v) HA-MA hydrogels were well defined, while collagen II nodules formed by cells in 0.5% (w/v) HS-MA hydrogels were more diffuse and less intense (FIG. 23A), especially in softer hydrogels made of 8% (w/v) PEGDMA.

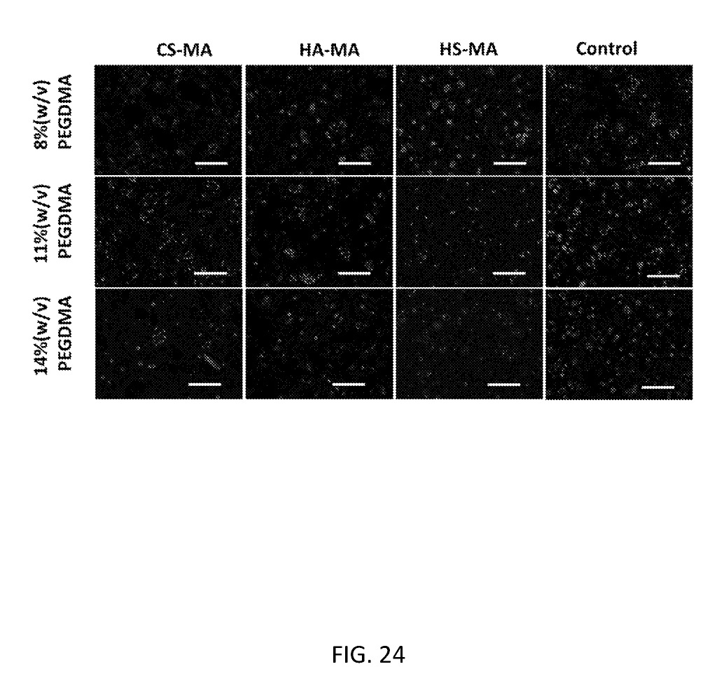

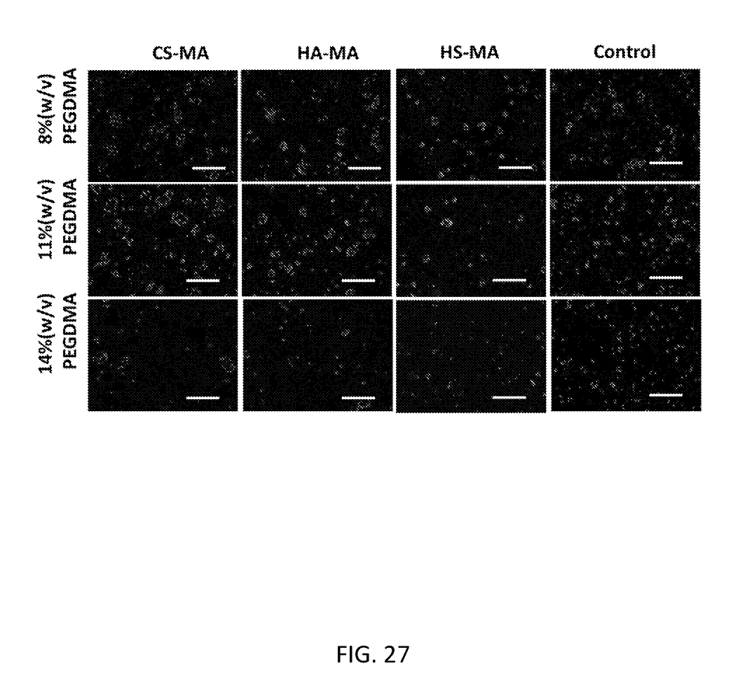

Immunostaining indicated that HS-MA appeared to promote the secretion of collagen I across all levels of mechanical stiffness (FIG. 24). Collagen I immunostaining was also more prominent in stiffer hydrogels containing 14% (w/v) PEGDMA (FIG. 24). Across all 39 hydrogel compositions, the levels of collagen X produced were far lower than the levels of collagens I and II produced (FIG. 27).

4 Discussion

In this study, we have demonstrated that the synergistic interaction between ADSCs and NChons can be modulated by biochemical and mechanical cues in a non-linear manner, using our combinatorial 3D hydrogel platform. Here, biochemical cues were provided by methacrylated ECM molecules (CS-MA, HA-MA, and HS-MA).

Mechanical stiffness was tuned by incorporating different amounts of PEGDMA into the hydrogels, yielding soft (˜15 kPa), moderately stiff (˜40 kPa), and stiff (˜100 kPa) matrices. PEGDMA was chosen for its bio-inert properties. Previously, we showed that modifying ECM molecules with a low number of methacrylate groups does not significantly affect hydrogel mechanical stiffness (Wang et al. (2014) Tissue Engineering Part A 20:2131-2139); thus, mechanical stiffness remained largely controlled by the amount of PEGDMA within the hydrogel (FIG. 19). This critical decoupling enables analysis of the effects of different types and concentrations of ECM on ADSC-NChon interaction without complications from the effects of mechanical stiffness.

Previous studies by our group demonstrated that ADSCs exhibit different chondrogenic gene-expression profiles when encapsulated in hydrogels with different types and concentrations of cartilage-specific ECM molecules (Wang et al., supra). Other groups reported similar observations: ECM-containing hydrogels direct stem-cell chondrogenesis by upregulating of expression of the genes encoding Sox9, collagen II, and aggrecan (Chung C, Burdick J A. Influence of three-dimensional hyaluronic acid microenvironments on mesenchymal stem cell chondrogenesis. Tissue engineering Part A. 2009; 15:243-54; Bosnakovski et al. (2006) Biotechnol Bioeng 93:1152-1163; Varghese et al. (2008) Matrix Biol 27:12-21). Despite their strong expression of chondrogenic genes, ADSCs alone are unable to produce significant amounts of hyaline cartilage-specific matrix (Wang et al., supra), prohibiting their sole use for effective clinical therapy. Importantly, co-cultures of stem cells and primary chondrocytes previously led to enhancements in cartilage-specific matrix production (Meretoja et al. (2012) Biomaterials 33:6362-6369; Lai et al. (2013) Scientific Reports. 3:3553; Wu et al. (2011) Tissue Engineering Part A 17:1425-1436; Yang H N, Park J S, Na K, Woo D G, Kwon Y D, Park K H. The use of green fluorescence gene (GFP)-modified rabbit mesenchymal stem cells (rMSCs) co-cultured with chondrocytes in hydrogel constructs to reveal the chondrogenesis of MSCs (Yang et al. (2009) Biomaterials 30:6374-6385). In particular, when co-cultured in 3D biomimetic hydrogels, stem cells such as ADSCs stimulated NChons via paracrine signaling to lay down cartilage-specific matrix (Meretoja et al. (2012) Biomaterials 33:6362-6369; Lai et al. (2013) Scientific Reports 3:3553; Liu et al. (2010) Biomaterials 31:9406-9414). A variety of platforms, such as cell-pellet cultures (Acharya et al. (2012) J Cell Physiol 227:88-97; Wu et al. (2011) Tissue Engineering Part A 17:1425-1436; Giovannini et al. (2010) Eur Cell Mater 20:245-59), hyaluronic acid scaffolds (Bian et al. (2011) Tissue Engineering Part A 17:1137-45), fibrin gels (Leyh et al. (2014) Stem Cell Res Ther 5:77), polylactic acid/polyglycolic acid scaffolds (Liu et al. (2010) Biomaterials 31:9406-9414), and PEG scaffolds (Lai et al. (2013) Scientific Reports 3:3553) have been used in chondrogenic co-culture studies. Here, we sought to investigate how ADSC-NChon interactions and hence matrix deposition are modulated by biochemical and mechanical cues provided by 3D biomimetic hydrogels.

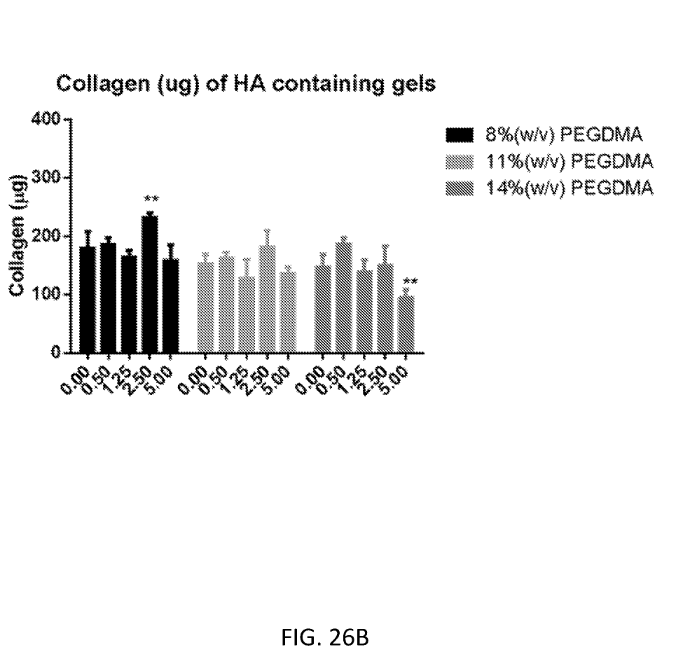

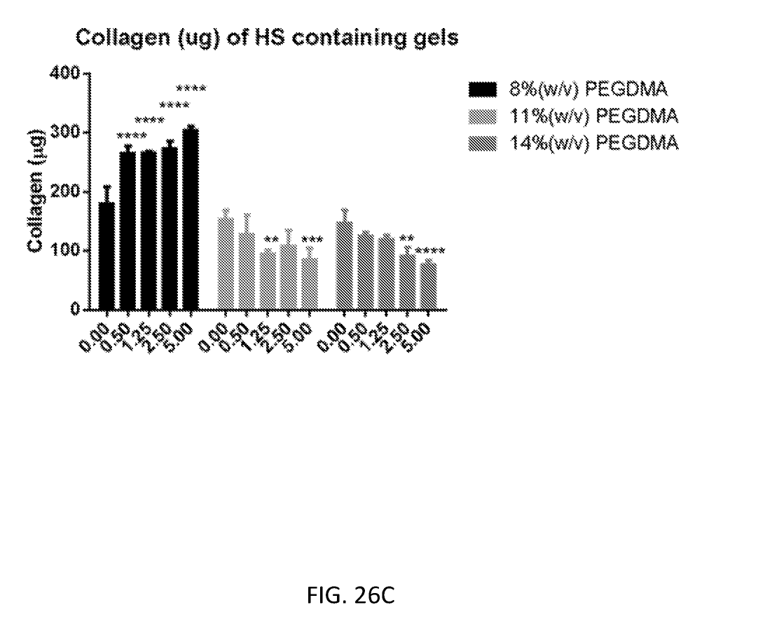

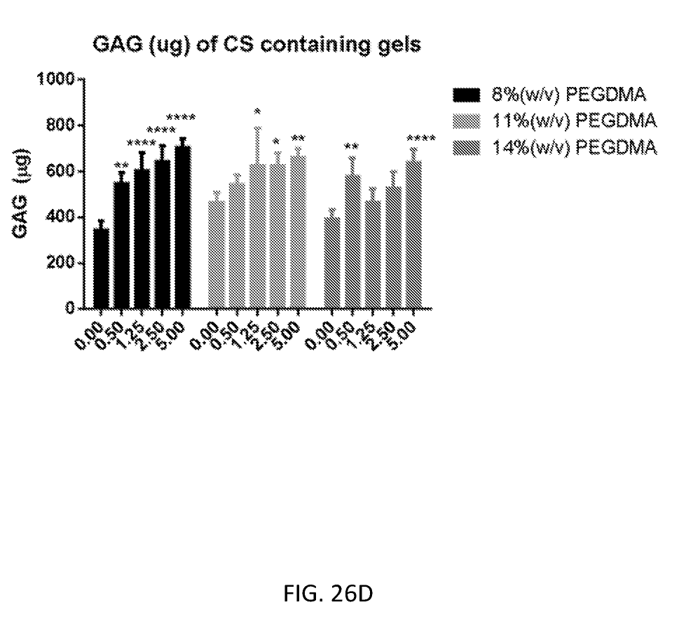

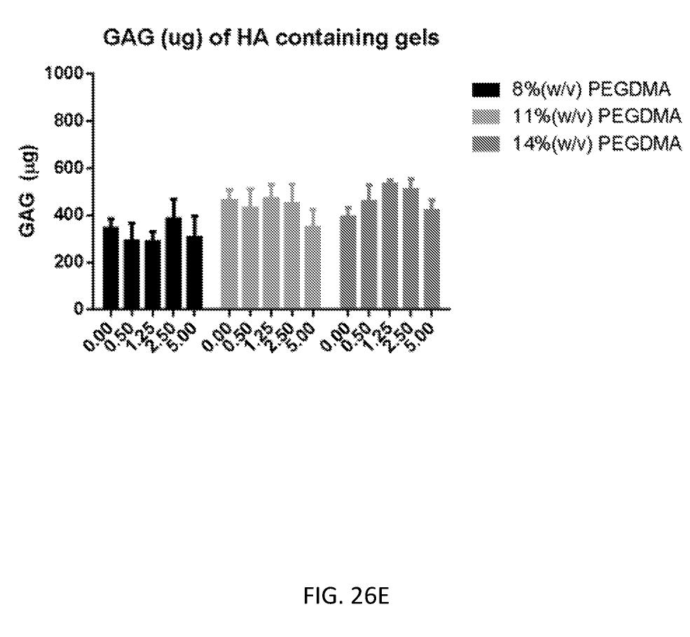

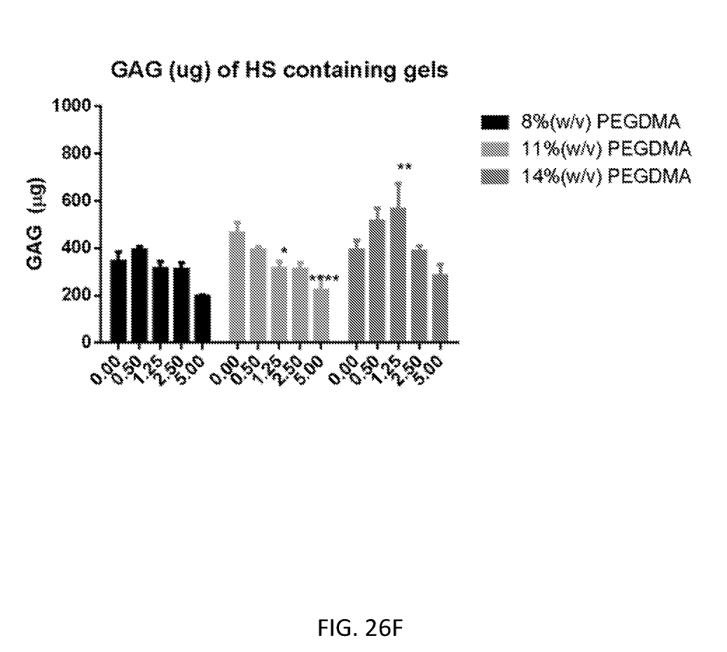

Our results demonstrated that while specific ECM molecules modulated cartilage-specific matrix formation in distinct fashions, only modest dose dependency was observed for each ECM species. At all levels of mechanical stiffness examined here (15-100 kPa), higher dosages of CS-MA stimulated cartilage-specific matrix synthesis (FIGS. 20D-20I; FIGS. 26A and 26D). Many other groups have used chondroitin sulfate in hydrogels to promote chondrogenesis in stem cells (Lai et al., supra; Varghese et al. (2008) Matrix Biol 27:12-21; Guo et al. (2012) J Mater Sci Mater Med 23:2267-7229; Wang et al. (2007) Nature Materials. 6:385-392). In addition, CS-containing hydrogels were previously more potent than collagen I- and hyaluronic acid-containing hydrogels in upregulating matrix secretion by chondrocytes (Hwang et al. (2007) FEBS Letters 581:4172-4178).

HA-MA is another commonly used cartilage-specific ECM with demonstrated efficacy in directing chondrogenesis (Chung et al. (2009) Tissue Engineering Part A 15:243-254; Kim et al. (2013) Biomaterials 34:5571-5580; Toh et al. (2012) Biomaterials. 33:3835-3845). Other groups have reported that the potency of HA-MA on stem-cell chondrogenesis is dependent on dosage (Erickson et al. (2009) Osteoarthritis Cartilage 17:1639-1648; Bian et al. (2013) Biomaterials 34:413-421). However, in those studies, increasing levels of HA-MA led to corresponding increases in mechanical stiffness, making it difficult to determine the relative effects of biochemical and mechanical cues. In our system, we increased the concentration of HA-MA up to 5% (w/v) with minimal impact on mechanical stiffness (FIG. 19). Within the range of matrix stiffness and HA-MA concentrations explored here, there was no significant dose dependency (FIGS. 20D-20I; FIGS. 26B and 26E). While there was a modest increase in sGAG production by cells in HA-MA-containing hydrogels when the mechanical stiffness was increased from ˜15 kPa to ˜40 kPa, no further increase in sGAG production occurred when the stiffness as increased to ˜100 kPa. Our findings may differ from the observations of other groups due to the different molecular weights of hyaluronic acid used, which is known to influence chondrocyte activity and therefore matrix secretion (Akmal et al. (2005) J Bone Joint Surg Br 87:1143-1149; Responte et al. (2012) J R Soc Interface 9:3564-3573; Chung et al. (2006) J Biomed Mater Res A 77(3):518-525). We used 20 kDa HA-MA, while other groups used longer-chain hyaluronic acids of up to 74 kDa within hydrogels and 2.7 MDa if delivered exogenously (Akmal et al., supra; Responte et al., supra; Chung et al., supra).

Here, the effects of biochemical cues provided by HS-MA were modulated by mechanical stiffness. In soft hydrogels (8% (w/v) PEGDMA, ˜15 kPa) increasing the dosage of HS-MA from 0.5% to 5% supported an increase in collagen matrix deposition. However, in moderately stiff (11% (w/v) PEGDMA, ˜40 kPa) and stiff (14% (w/v) PEGDMA, ˜100 kPa) hydrogels, this increase in HS-MA dosage led to decreases in collagen matrix deposition. HS-MA consistently prompted more collagen I matrix production than did CS-MA and HA-MA across all levels of stiffness (FIG. 24). This high level of collagen I accumulation is undesirable in cartilage engineering because fibrocartilage, which is softer and less smooth than hyaline cartilage, is characterized by a high deposition of collagen I (Eyre et al. (1983) FEBS Lett 158:265-270). Thus, our HS-MA-containing hydrogels may be more suitable for fibrocartilage or meniscus regeneration, for which collagen I is desirable (Makris et al. (2011) Biomaterials 32:7411-7431). Further, immunostaining revealed that collagen II nodules have defined edges in both CS-MA- and HA-MA-containing hydrogels but were more diffuse in HS-MA-containing hydrogels, even at 0.5% (w/v) HA-MA (FIGS. 22A and 23A); perhaps cell degrade HS-MA differently than they do CS-MA and HA-MA. Cells may be more efficient in degrading HS-MA, enabling secreted matrix to diffuse throughout the hydrogel, resulting in more diffuse nodules with no defined edges. This scenario may explain the low matrix accumulation in HS-MA-containing hydrogels after 21 days in chondrogenic conditions, despite HS-MA's ability to bind growth factors: secreted matrix may have diffused out of the hydrogels.

Native cartilage is zonally organized, with collagen levels decreasing gradually while sGAG levels increase gradually from the superficial zone to the deep zone. It remains challenging to direct cells to secrete matrix in a manner that faithfully reproduces this zonal structure. The present investigation can be used to guide the design of zonally organized cartilage constructs. Future work will include the incorporation of specific ECM molecules into hydrogels in distinct spatial zones in order to direct cells to secrete cartilage-specific matrix that mimics the zones of native cartilage.

5 Conclusions

Here we report the use of combinatorial hydrogels with decoupled biochemical and mechanical properties to study the interactions between ADSCs and NChons. Our combinatorial platform allows the addition of biomimetic methacrylated ECM molecules with few effects on hydrogel mechanical stiffness, which is solely controlled by the amount of PEGDMA in the hydrogel. We verified that synergistic matrix production by a mixed culture of ADSCs and NChons was modulated by ECM molecules and mechanical cues in a non-linear manner. In particular, at all levels of mechanical stiffness examined here, CS-MA consistently led to the production of high amounts of collagen and sGAG in a dose-dependent manner, making it a desirable choice for cartilage tissue engineering. Insights into how biochemical cues and mechanical stiffness affect ADSC-NChon interactions in terms of collagen matrix production and cartilage nodule formation will guide the future development of an in vitro zonally organized cartilage construct.

While the preferred embodiments of the invention have been illustrated and described, it will be appreciated that various changes can be made therein without departing from the spirit and scope of the invention.

What is claimed is:

1. A method for treating a patient for cartilage damage or loss, the method comprising:
a) combining chondrocytes with adipose-derived stem cells in a mixed culture, wherein the mixed culture comprises 1% to 25% chondrocytes and 75% to 99% adipose-derived stem cells;
b) adding the mixed culture to a hydrogel composition comprising chondrogenic media, TGF-β3, and at least one extracellular matrix molecule selected from the group consisting of chondroitin sulfate methacrylate (CS-MA), hyaluronic acid methacrylate (HA-MA), and heparan sulfate methacrylate (HS-MA), wherein the hydrogel composition has a Young's modulus of from about 3 kPa to about 100 kPa; and
c) transplanting the hydrogel composition comprising the mixed culture to the patient at a site in need of cartilage replacement.

2. The method of claim 1, wherein cartilage comprising nodules having a nodule size of at least 100 μm in length is produced from the hydrogel composition in the subject.

3. The method of claim 1, wherein the hydrogel composition comprises a polyethylene glycol (PEG)-based hydrogel.

4. The method of claim 3, wherein the PEG-based hydrogel comprises poly(ethylene glycol) diacrylate (PEGDA) or poly(ethylene glycol) dimethacrylate (PEGDMA).

5. The method of claim 4, wherein the hydrogel composition comprises PEGDMA at a concentration ranging from about 8% (w/v) to about 14% (w/v).

6. The method of claim 1, wherein the hydrogel composition comprises a photopolymerizable hydrogel.

7. The method of claim 1, wherein the at least one extracellular matrix molecule is at a concentration ranging from about 0.5% (w/v) to about 5% (w/v).

8. The method of claim 1, wherein the mixed culture comprises 1% to 2% chondrocytes and 98% to 99% adipose-derived stem cells.

9. The method of claim 1, wherein the mixed culture comprises 5% to 25% chondrocytes and 95% to 75% adipose-derived stem cells.

10. The method of claim 9, wherein the mixed culture comprises 10% to 25% chondrocytes and 90% to 75% adipose-derived stem cells.

11. The method of claim 1, wherein the cartilage nodules comprise articular hyaline cartilage.

12. The method of claim 1, wherein the Young's modulus is in a range of 15 kPa-100 kPa.

13. The method of claim 1, further comprising culturing the chondrocytes ex vivo in the hydrogel composition in the mixed culture with the adipose-derived stem cells before transplanting the hydrogel composition to the subject.

14. The method of claim 13, wherein transplanting the hydrogel composition occurs after culturing the chondrocytes ex vivo in the hydrogel composition in the mixed culture with the adipose-derived stem cells for at least one week.

15. The method of claim 14, wherein transplanting the hydrogel composition occurs after culturing the chondrocytes ex vivo in the hydrogel composition in the mixed culture with the adipose-derived stem cells for at least two weeks.

16. The method of claim 15, wherein transplanting the hydrogel composition occurs after culturing the chondrocytes ex vivo in the hydrogel composition in the mixed culture with the adipose-derived stem cells for at least three weeks.

17. The method of claim 13, wherein the chondrocytes in the transplanted hydrogel composition produce additional cartilage in vivo in the patient.

18. The method of claim 2, wherein the cartilage comprises articular hyaline cartilage.

19. The method of claim 2, wherein the cartilage is produced in vivo under hypoxic conditions.

20. The method of claim 19, where the hypoxic conditions have a local O₂ tension ranging from 1% to 7%.

21. The method of claim 1, wherein the site is at a damaged joint.

22. The method of claim 1, wherein the subject has a traumatic injury or a disease involving cartilage degeneration.

23. The method of claim 22, wherein the disease involving cartilage degeneration is arthritis.

24. The method of claim 1, further comprising administering and effective amount of TGF-β3 to the patient.

25. The method of claim 1, wherein the chondrocytes are derived from the patient.

26. The method of claim 1, wherein the chondrocytes are from a matched donor.

27. The method of claim 1, wherein the chondrocytes are allogeneic.

28. A method for producing cartilage, the method comprising:
a) obtaining chondrocytes from a subject;
b) combining the chondrocytes with adipose-derived stem cells in a mixed culture, wherein the mixed culture comprises 1% to 25% chondrocytes and 75% to 99% adipose-derived stem cells;
c) adding the mixed culture to a hydrogel composition, wherein the hydrogel composition has a Young's modulus of from about 3 kPa to about 100 kPa;
d) culturing the chondrocytes ex vivo or in vivo in the hydrogel composition, wherein the chondrocytes are cultured in the mixed culture with the adipose-derived stem cells in chondrogenic media comprising TGF-β3 and at least one extracellular matrix molecule selected from the group consisting of chondroitin sulfate methacrylate (CS-MA), hyaluronic acid methacrylate (HA-MA), and heparan sulfate methacrylate (HS-MA) under conditions, whereby cartilage is produced comprising nodules having a nodule size of at least 100 μm in length.

29. The method of claim 28, wherein the mixed culture comprises 1% to 2% chondrocytes and 98% to 99% adipose-derived stem cells.

30. A hydrogel composition comprising cartilage prepared by the method of claim 28.
